(19) United States
(12) Patent Application Publication
(10) Pub. No.: US 2023/0416785 A1
(43) Pub. Date: Dec. 28, 2023

(54) NUCLEASE AND APPLICATION THEREOF

(71) Applicant: CRISP-HR Therapeutics, Inc., San Carlos, CA (US)

(72) Inventor: Christopher Hackley, San Carlos, CA (US)

(21) Appl. No.: 18/170,506

(22) Filed: Feb. 16, 2023

Related U.S. Application Data
(63) Continuation of application No. PCT/US2021/047208, filed on Aug. 23, 2021.
(60) Provisional application No. 63/163,619, filed on Mar. 19, 2021.
(60) Provisional application No. 63/069,634, filed on Aug. 24, 2020.

(51) Int. Cl. C12N 15/90; C12N 15/11; C12N 9/22

(52) U.S. Cl. CPC C12N 15/907; C12N 15/11; C12N 9/22; C12N 2310/20; C12N 2800/80

(57) ABSTRACT

Described herein are compositions comprising a nuclease. Also described herein are methods utilizing the compositions comprising the nuclease.

### CROSS-REFERENCE

This application is a continuation of International Application No. PCT/US2021/047208, filed on Aug. 23, 2021, which claims priority to U.S. provisional application No. 63/069,634, filed on Aug. 24, 2020, and U.S. provisional application No. 63/163,619, filed on Mar. 19, 2021, the entirety of which are hereby incorporated by reference herein.

### REFERENCE TO A SEQUENCE LISTING XML

The instant application contains a Sequence Listing which has been submitted electronically in XML format and is hereby incorporated by reference in its entirety. Said XML file, created on Feb. 15, 2023, is named 55190-704_301_SL.xml and is 386,357 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Targeted editing of nucleic acids is a highly promising approach for studying genetic functions and for treating and ameliorating symptoms of genetic disorders and diseases. Most notable target-specific genetic modification methods involve engineering and using of zinc finger nucleases (ZFNs), transcription activator like effector nucleases (TALENs), and RNA-guided DNA endonuclease Cas. Frequency of introducing mutations such as deletions and insertions at the targeted nucleic acids through the non-homologous end joining (NHEJ) repair mechanism limits the applications of genetic targeting and editing in the development of therapeutics.

### SUMMARY

Accordingly, there remains a need for genetic modification to decrease unwanted deletions and insertions. There also remains a need for genetic modification that can insert a repair template into a genome for treatment of a disease or condition, where the repair template comprises a length that is on a magnitude of kilobases. Such repair template length offers an improvement over genetic modification methods currently available, where the repair template described herein can: correct multiple mutations that are far apart; or encode a full-length transgene.

Described herein, in some aspects, is a method for introducing an edit into a genomic locus of a plurality of cells, the method comprising contacting the plurality of the cells with: a Cas fusion protein complex comprising a Cas fusion protein complexed with a guide polynucleotide configured to bind to the genomic locus of the cell; and a polynucleotide of interest comprising a nucleic acid donor sequence that is at least 1000 bp bp in length, where the Cas fusion protein complex is configured to induce homology-directed repair (HDR) of the genomic locus of a cell with the nucleic acid donor sequence to produce an edited cell. In some embodiments, the nucleic acid donor sequence comprises the nucleic acid sequence that is at least 2000 bp in length. In some embodiments, the nucleic acid donor sequence comprises the nucleic acid sequence that is at least 5000 bp in length. In some embodiments, the nucleic acid donor sequence comprises the nucleic acid sequence that is at least 10000 bp in length. In some embodiments, at least 50% of the plurality of cells remain viable after the genomic locus is edited with the nucleic acid donor sequence. In some embodiments, at least at least 60% of the plurality of cells remain viable after the genomic locus is edited with the nucleic acid donor sequence. In some embodiments, at least 70% of the plurality of cells remain viable after the genomic locus is edited with the nucleic acid donor sequence. In some embodiments, at least 80% of the plurality of cells remain viable after the genomic locus is edited with the nucleic acid donor sequence. In some embodiments, at least 90% of the plurality of cells remain viable after the genomic locus is edited with the nucleic acid donor sequence. In some embodiments, the Cas fusion protein comprises a Cas nuclease fused to an exonuclease or a fragment thereof. In some embodiments, the Cas fusion protein comprises a Cas9 nuclease. In some embodiments, the Cas9 nuclease comprises a polypeptide sequence that is at least 75% identical to SEQ ID NO: 7-23. In some embodiments, the Cas9 nuclease comprises a polypeptide sequence that is at least 80% identical to SEQ ID NO: 7-23. In some embodiments, the Cas9 nuclease comprises a polypeptide sequence that is at least 85% identical to SEQ ID NO: 7-23. In some embodiments, the Cas9 nuclease comprises a polypeptide sequence that is at least 90% identical to SEQ ID NO: 7-23. In some embodiments, the Cas9 nuclease comprises a polypeptide sequence that is at least 95% identical to SEQ ID NO: 7-23. In some embodiments, the Cas9 nuclease comprises a polypeptide sequence that is at least 99% identical to SEQ ID NO: 7-23. In some embodiments, the Cas fusion protein comprises a Cas12 nuclease. In some embodiments, the Cas12 nuclease comprises a polypeptide sequence that is at least 75% identical to SEQ ID NO: 55-57. In some embodiments, the Cas12 nuclease comprises a polypeptide sequence that is at least 80% identical to SEQ ID NO: 55-57. In some embodiments, the Cas12 nuclease comprises a polypeptide sequence that is at least 85% identical to SEQ ID NO: 55-57. In some embodiments, the Cas12 nuclease comprises a polypeptide sequence that is at least 90% identical to SEQ ID NO: 55-57. In some embodiments, the Cas12 nuclease comprises a polypeptide sequence that is at least 95% identical to SEQ ID NO: 55-57. In some embodiments, the Cas12 nuclease comprises a polypeptide sequence that is at least 99% identical to SEQ ID NO: 55-57. In some embodiments, the Cas fusion protein comprises the Cas nuclease fused to a Human Exo1 (hExo1). In some embodiments, the Cas fusion protein comprises the Cas nuclease fused a fragment of the hExo1. In some embodiments, the hExo1 comprises a polypeptide sequence that is at least 75% identical to SEQ ID NO: 1. In some embodiments, the hExo1 comprises a polypeptide sequence that is at least 80% identical to SEQ ID NO: 1. In some embodiments, the hExo1 comprises a polypeptide sequence that is at least 85% identical to SEQ ID NO: 1. In some embodiments, the hExo1 comprises a polypeptide sequence that is at least 90% identical to SEQ ID NO: 1. In some embodiments, the hExo1 comprises a polypeptide sequence that is at least 95% identical to SEQ ID NO: 1. In some embodiments, the hExo1 comprises a polypeptide sequence that is at least 99% identical to SEQ ID NO: 1. In some embodiments, the hExo1 comprises a polypeptide sequence that is at least 75% identical to SEQ ID NO: 2. In some embodiments, the hExo1 comprises a polypeptide sequence that is at least 80% identical to SEQ ID NO: 2. In some embodiments, the hExo1 comprises a polypeptide sequence that is at least 85% identical to SEQ ID NO: 2. In some embodiments, the hExo1 comprises a polypeptide sequence that is at least 90% identical to SEQ ID NO: 2. In some embodiments, the hExo1 comprises a polypeptide sequence that is at least 95% identical to SEQ ID NO: 2. In some embodiments, the hExo1 comprises a polypeptide sequence that is at least 99% identical to SEQ ID NO: 2. In some embodiments, the hExo1 comprises a polypeptide sequence of SEQ ID NO: 2. In some embodiments, the Cas9 fusion protein comprises the Cas fused to a DNA replication ATP-dependent helicase/nuclease (DNA2). In some embodiments, the Cas9 fusion comprises the Cas fused to fragment of the DNA2. In some embodiments, the DNA2 comprises a polypeptide sequence that is at least 75% identical to SEQ ID NO: 4. In some embodiments, the DNA2 comprises a polypeptide sequence that is at least 80% identical to SEQ ID NO: 4. In some embodiments, the DNA2 comprises a polypeptide sequence that is at least 85% identical to SEQ ID NO: 4. In some embodiments, the DNA2 comprises a polypeptide sequence that is at least 90% identical to SEQ ID NO: 4. In some embodiments, the DNA2 comprises a polypeptide sequence that is at least 95% identical to SEQ ID NO: 4. In some embodiments, the DNA2 comprises a polypeptide sequence that is at least 99% identical to SEQ ID NO: 4. In some embodiments, the DNA2 comprises a polypeptide sequence that is at least 75% identical to SEQ ID NO: 5. In some embodiments, the DNA2 comprises a polypeptide sequence that is at least 80% identical to SEQ ID NO: 5. In some embodiments, the DNA2 comprises a polypeptide sequence that is at least 85% identical to SEQ ID NO: 5. In some embodiments, the DNA2 comprises a polypeptide sequence that is at least 90% identical to SEQ ID NO: 5. In some embodiments, the DNA2 comprises a polypeptide sequence that is at least 95% identical to SEQ ID NO: 5. In some embodiments, the DNA2 comprises a polypeptide sequence that is at least 99% identical to SEQ ID NO: 5. In some embodiments, the DNA2 comprises a polypeptide sequence of SEQ ID NO: 5. In some embodiments, the repair template comprises a mutated protospacer adjacent motif (PAM) sequence located at the immediate 3′ end of a cleavage site, wherein said mutated PAM sequence comprises 5′-NCG-3′ or 5′-NGC-3′. In some embodiments, the mutated PAM sequence is not cleaved by the Cas fusion protein. In some embodiments, the repair template is a single-stranded DNA. In some embodiments, the repair template is a double-stranded DNA. In some embodiments, the method comprises an exogenous polynucleotide encodes both the Cas fusion protein and the guide polynucleotide. In some embodiments, the method comprises an exogenous polynucleotide encodes the Cas fusion protein, the guide polynucleotide, and the repair template. In some embodiments, the exogenous polynucleotide comprises a nucleic acid sequence that is at least 75% identical to SEQ ID NOs: 81-86. In some embodiments, the exogenous polynucleotide comprises a nucleic acid sequence that is at least 80% identical to SEQ ID NOs: 81-86. In some embodiments, the exogenous polynucleotide comprises a nucleic acid sequence that is at least 85% identical to SEQ ID NOs: 81-86. In some embodiments, the exogenous polynucleotide comprises a nucleic acid sequence that is at least 90% identical to SEQ ID NOs: 81-86. In some embodiments, the exogenous polynucleotide comprises a nucleic acid sequence that is at least 95% identical to SEQ ID NOs: 81-86. In some embodiments, the exogenous polynucleotide comprises a nucleic acid sequence that is at least 99% identical to SEQ ID NOs: 81-86. In some embodiments, the exogenous polynucleotide comprises a nucleic acid sequence that is SEQ ID NOs: 81-86. In some embodiments, the genomic locus encodes a gene associated with the cancer. In some embodiments, the gene associated with the cancer is an oncogene. In some embodiments, the gene associated with the cancer is a tumor suppressor gene. In some embodiments, the gene associated with the cancer is Cadherin. In some embodiments, the gene associated with the cancer is E-Cadherin. In some embodiments, the gene associated with the cancer is Catenin. In some embodiments, the gene associated with the cancer is beta-Catenin. In some embodiments, the genomic locus comprises at least one mutation. In some embodiments, the genomic locus comprises a safe harbor site (SHS). In some embodiments, at least one repair template is inserted into the SHS. In some embodiments, at least two repair templates are inserted into the SHS. In some embodiments, the Cas fusion protein increases HDR editing rate in the plurality of the cells compared to a HDR editing rate induced by a second Cas protein in a comparable plurality of cells. In some embodiments, the Cas fusion protein increases the HDR editing rate in the plurality of the cells by at least 10% or more compared to the HDR editing rate induced by a second Cas protein in a comparable plurality of cells. In some embodiments, the Cas fusion protein increases the HDR editing rate in the plurality of the cells by at least 50% or more compared to the HDR editing rate induced by a second Cas protein in a comparable plurality of cells. In some embodiments, the Cas fusion protein increases the HDR editing rate in the plurality of the cells by at least 100% or more compared to the HDR editing rate induced by a second Cas protein in a comparable plurality of cells. In some embodiments, the Cas fusion protein increases the HDR editing rate in the plurality of the cells by at least 200% or more compared to the HDR editing rate induced by a second Cas protein in a comparable plurality of cells. In some embodiments, the Cas fusion protein increases the HDR editing rate in the plurality of the cells by at least 300% or more compared to the HDR editing rate induced by a second Cas protein in a comparable plurality of cells. In some embodiments, the Cas fusion protein decreases endogenous p53 activity in the plurality of the cells compared to an endogenous p53 activity induced by a second Cas protein in a comparable plurality of cells. In some embodiments, the Cas fusion protein decreases endogenous p53 activity in the plurality of the cells by at least 10% or more compared to an endogenous p53 activity induced by a second Cas protein in a comparable plurality of cells. In some embodiments, the Cas fusion protein decreases endogenous p53 activity in the plurality of the cells by at least 50% or more compared to an endogenous p53 activity induced by a second Cas protein in a comparable plurality of cells. In some embodiments, the Cas fusion protein decreases endogenous p53 activity in the plurality of the cells by at least 100% or more compared to an endogenous p53 activity induced by a second Cas protein in a comparable plurality of cells. In some embodiments, the Cas fusion protein decreases endogenous p53 activity in the plurality of the cells by at least 200% or more compared to an endogenous p53 activity induced by a second Cas protein in a comparable plurality of cells. In some embodiments, the Cas fusion protein decreases endogenous p53 activity in the plurality of the cells by at least 300% or more compared to an endogenous p53 activity induced by a second Cas protein in a comparable plurality of cells. In some embodiments, the second Cas protein is a wild type Cas9 nuclease.

Described herein, in some aspects, is a pharmaceutical formulation comprising one or more of: the Cas fusion protein complex of any one of the above claims and the repair template of any one of the above claims. In some embodiments, the pharmaceutical composition comprises a pharmaceutically acceptable excipient.

Described herein, in some aspects, is a system comprising one or more of: the Cas fusion protein complex described herein, and the repair template described herein, or the pharmaceutical formulation described herein.

Described herein, in some aspects, is a kit comprising one or more of: the Cas fusion protein complex described herein, and the repair template described herein, the pharmaceutical formulation described herein, or the system described herein. In some embodiments, the kit comprises instructions for carrying out a method described herein.

### BRIEF DESCRIPTION OF THE DRAWINGS

This patent application contains at least one drawing executed in color. Copies of this patent or patent application with color drawing(s) will be provided by the Office upon request and payment of the necessary fee.

FIGS. 1A-D illustrate initial construction and characterization of Cas9-HRs. FIG. 1A. Diagram showing fusions of Cas9-HRs 1-9, with Cas9, NLS sequences, hExo1, and peptide linkers being black lines. Sequences of peptide linkers used are available in SEQ ID NOs: 211-215. FIG. 1B. Top: Diagram of the px330 plasmid used as the expression vector for Cas9 and Cas9-HRs; Bottom: example of a 96 well seeding pattern for standard A549 toxicity assays used throughout the paper. All experiments contained at least two independent replicates. FIG. 1C. Cas9-HRs reduce cellular toxicity in A549 cells. The target site on Chromosome 12 is depicted above graph showing cellular toxicity for Cas9-HRs 1-8 (1-8), Cas9, Cas9+hExo1, and untransfected controls (Con). Fluorescence values were normalized to untransfected control values, and all Cas9-HRs and GFP show significant reductions in cellular toxicity relative to Cas9 or Cas9+hExo1 (p<<0.0001, n=16, two-sided students t-test, error bar=SEM). FIG. 1D. Pifithrin-α treatment reduces Cas9 mediated cellular toxicity compared to treatment with either solvent (n=16, p<0.001 two-sided students t-test, error bar=SEM), whereas Pifithrin-α treatment in Cas9-HR transfected cells does not affect cellular toxicity relative to DMSO treated controls (light gray, n=8, p>0.05, two-sided students t-test, error bar=SEM).

FIG. 2A-C illustrate Cas9-HR 8 decreasing cellular toxicity and increasing HDR. FIG. 2A. Diagram of the H2B-mNeon repair template, as well as showing the location of hH2B-G4 guide polynucleotide. Light gray: silent mutations introduced in RT sequence, black lines: surrounding genomic sequence. FIG. 2B. Graph of cellular toxicity of Cas9-HRs 4,5,6,8, Cas9 and untransfected controls (Con) targeting H2B-G4 in A549 cells. Only Cas9-HR shows a significant reduction in toxicity compared to Cas9 (p<0.001, n=16, two-sided students t-test, error bar=SEM). FIG. 2C. Quantification of Cas9-HR 4,8 and Cas9 HDR rate in K562 cells. Top left and right show quantification of HDR rate; Cas9-HR 8 shows significantly higher HDR rates than either Cas9-HR 4 or Cas9 (two independent replicates, n=150-200 cells per replicate, p<0.005 two-sided students t-test, error bar=SEM). Bottom shows raw images, with Bright Field (left), mNeon (middle), and both merged (right), with white arrows showing nuclear mNeon fluorescence.

FIG. 3A-C illustrate Cas9-HR 8 showing decreased toxicity and increased HDR rates in an independent assay. FIG. 3A. Left, diagram showing the Puromycin RT template, guide polynucleotides Int-G2 and G3, CMV promoter, PuroR CDS, SV40 polyA sequence, guide targets, and surrounding genomic sequence. Right, graph showing cellular toxicity of Cas9-HRs 4,8, Cas9, PuroR RT and untransfected controls (Con) in A549 cells. Both Cas9-HR 4 and 8 show significant reductions in toxicity relative to Cas9 (p<<0.001, n=16, two-sided student t-test, error bar=SEM). FIG. 3B. Experimental protocol to measure HDR rate of Cas9-HR and Cas9 in K562 cells. FIG. 3C. Top, Diagram showing successful integration of PuroR RT transgene, left and right primer pair shown. Left, bottom shows cellular viability normalized to transfection with a plasmid containing the puromycin RT. Cas9-HR 8 shows a roughly two fold increase in cellular viability compared to Cas9 with both Int-G2 or G3 (p<0.005, n=8 for each treatment, student two-sided t-test, error bar=SEM). Bottom right, specific amplification is seen for both Cas9-HR 8 and Cas9, but not cells transfected with GFP or untransfected cells (Con).

FIG. 4A illustrates diagram of AAVS1 Renella Luciferase Repair Template (RLucRT). Diagram showing the design of the AAVS1 region on human Chromosome 19. AAVS1 Right and left homology arms shown in white and denoted by 5′ and 3′; the strong synthetic promoter CAG; Renella Luciferase ORF; bGH poly adenylation sequence; AAVS1 guide RNAs G1 and G2 (corresponding to T1 and T2 in Mali et al. 2013) shown in red. Not shown, mutations introduced into repair template to prevent cutting of successfully integrated RT by Cas9.

FIG. 4B illustrates experimental procedures for RLucRT experiments. Diagram showing experimental procedure for the dsDNA RLucRT HDR assay in H1299 cells. H1299 are transfect with either Cas9-HR 8 or Cas9 (NT) targeting either G1 or G2. After two days, cellular viability is quantified via Resazurin assay, then cells are washed with PBS then lysed. Lysate is then incubated with Coelenterazine, and luminescence immediately quantified via plate reader.

FIG. 4C illustrates H1299 cellular viability, showing H1299 cell viability normalized to cells transfected with RLucRT template alone. As expected, due to the lack of p53 pathway activity in H1299 cells, no significant differences in viability were seen between Cas9-HR 8 and Cas9, though both were slightly reduced compared to RT alone.

FIG. 4D (left) illustrates background subtracted raw Luminescence Readings from Cas9-HR 8 and Cas9 cells. Graph showing background (untransfected cells) subtracted luminescence readings from both Cas9-HR 8 G1 and G2, Cas9 G1 and G2 and RT transfected cells. Both Cas9-HR 8 and Cas9 G1 show significantly higher luminescence readings than G2, with Cas9-HR 8 showing significantly higher values than either Cas9 when comparing between G1 and G2. Additionally, though the RLucRT in theory can drive expression from non-integrated RTs, in practice the dsDNA RT cannot drive significant expression, as shown by luminescence values virtually indistinguishable from background readings. FIG. 4D (right) illustrates Cas9 normalized luminescence, showing Cas9-HR and Cas9 luminescence values for each individual guide polynucleotide (i.e. Cas9-HR8 to Cas9 G1 or G2). As seen with other integration assays, Cas9-HR drives significant increases in HDR rates (2-3×) for both G1 and G2 compared to Cas9.

FIG. 4E illustrates junction PCR of AAVS1 RLucRT integrations. DNA was extracted from H1299 cells and nested PCR was performed to amplify both 5′ and 3′ junctions as diagrammed using primers specific for genomic and RLucRT sequences. Specific amplification was seen for all samples except for untransfected controls (bottom, 8-G2 and NT-G2 performed, but data not shown), indicating successful genomic integration of the RLucRT transgene. Diagram shows genomic prediction of RLucRT at the AAVS1 locus. Nested PCR was performed on both the 5′ and 3′ ends, with both sets of primers being specific for the genome and repair template respectively (5′ F1, F2 and 3′ R1, R2 for the genome; 5′ R1, R2 and 3′ F1, F2 for RlucRT). Agarose gel (bottom left) illustrates nested PCR of 5′ RLucRT from genomic DNA. Cells transfected with either Cas9-HR 8 plus RlucRT (8-G1) or Cas9 plus RlucRT (NT-G1) targeting AAVS1-G1, RlucRT (RT) only or non-transfected (con). Strong specific amplification can be seen for 8-G1, NT-G1 and RT, but not in control samples. Given the extremely sensitive nature of nested PCR, it is not surprising that some integrant were detected in the RT only treatment. Agarose gel (bottom right) illustrates nested PCR of 3′ RLucRT from genomic DNA. Cells transfected with either Cas9-HR 8 plus RlucRT (8-G1) or Cas9 plus RlucRT (NT-G1) targeting AAVS1-G1, RlucRT (RT) only or non-transfected (con). Strong specific amplification can be seen for 8-G1, NT-G1 and RT, but not in control samples. Given the extremely sensitive nature of nested PCR, it is not surprising that some successful integrants were detected in the RT only treatment.

FIGS. 5A-C illustrate enzymatically active Cas9-HRs purified from E. coli. FIG. 5A illustrates SDS-PAGE gel of purified Cas9-HRs 3, 4, and 8. Cas9-HRs 3 and 4 may undergo some proteolytic cleavage during expression/purification, however all three have upper bands which run at the predicted full-length size (˜200 kD) of Cas9-HR. FIG. 5B illustrates exonuclease activity assays for purified Cas9-HRs. The top diagram shows the HBB genomic region, and the location of primers used to amplify the amplicon used in following nuclease assays. 30 nM of Cas9-HRs 3, 4, 8 and Cas9 and control reactions were incubated with 3 nM of the purified HBB amplicon, and incubated at 37° C. for 60 minutes, after which 1 μL of proteinase K (200 μg/mL) was added and reactions incubated at 65° C. for an additional 20 minutes. Reactions were then electrophoresed and visualized on an agarose gel. FIG. 5C illustrates quantification of exonuclease activity. Quantification of exonuclease activity assayed in FIG. 5B. 3 replicates were performed for each reaction and were quantified via FIJI (ImageJ) and then normalized to control amplicon levels. Cas9-HRs 3 and 8 show significant exonuclease activity, whereas neither Cas9-HR 4 or Cas9 show no significant activity. During purification it was noted that Cas9-HR 4 required somewhat different conditions for successful purification, and may require different reaction conditions than Cas9-HRs 3 and 8.

FIGS. 6A-D illustrate Cas9 mediated cellular toxicity correlated with NHEJ repair pathway activation in A549 cells. FIG. 6A. Left, diagram of Human HBB exons 1, 2 and surrounding genomic sequence. Guide polynucleotides G1, G2, G3 shown with arrows, Exons 1 and 2, surrounding genomic sequences: black. Right, cellular viability of A549 cells transfected with Cas9 targeting either HBB-G1, G2, or G3 (error bar=SEM). FIG. 6B. Sequencing trace of HBB exon1 amplified from Cas9 HBB-G3 transfected cells. FIG. 6B discloses SEQ ID NO: 223. Bar shows HBB-G3, showing the characteristic pattern indicating NHEJ repair. FIG. 6C. Cellular viability of A549 cells transfected with Cas9-HRs 1-9 (1-9,), Cas9 (10,), Cas9+hExo1(11,), GFP (12,), or untransfected controls (Con). Cas9-HRs 1-4, and 6-8 show significant reductions in cellular toxicity compared to Cas9 or Cas9+hExo1, p<0.01, two-sided students t-test, error bar=SEM. FIG. 6D. A549 cells transfected with Cas9 treated with a dilution series of Pifithrin-α. Cells treated with 10 nM-10 μM Pifithrin-α show decreasing levels of toxicity with increasing concentrations of Pifithrin-α, consistent with a specific dose-dependent response to p53 pathway inhibition.

FIG. 7A-D illustrate Cas9-HRs showing similar expression and localization as Cas9. FIG. 7A (top gel) illustrates anti-Cas9 western blot of K562 cells transfected with Cas9-HR 4-8 #2. Additional anti-Cas9 western blot of Cas9-HRs 4-8, again showing predicted size of ˜200 kD. Western blot specificity additionally shown by reduced intensity of staining of Cas9-HR 4 compared to others. FIG. 7A (bottom gel) illustrates anti-Cas9 western blot of K562 cells transfected with Cas9-HRs 4-8 #3. Additional anti-Cas9 western blot of Cas9-HRs 4-8, again showing predicted size of ˜200 kD. Western blot specificity additionally shown by reduced intensity of staining of Cas9-HR 4 compared to others. FIG. 7B. Images of Cas9-HR 4, 8, Cas9 (NT) transfected or untransfected control (Con) K562 cells stained for Cas9 expression. Strong localization of Cas9-HRs 4,8 and Cas9 can be seen in the nucleus (white arrows), whereas control cells only show weak and diffuse signal. FIG. 7C illustrates anti-Cas9 western blot of Cas9-HR 8, Cas9 and Untransfected K562 cells. Cas9-HR 8 and Cas9 show strong staining at expected sizes of ˜200 kD and 160 kD respectively, the size shift seen with Cas9-HR 8 further confirms fusion of Exo1 nuclease domain. Finally, high molecular weight staining is specific for Cas9-HR and Cas9, as control cells do not show staining in this size range. FIG. 7D illustrates anti-Cas9 western blot of Purified Cas9-HR 3 and Cas9. Western blot of purified Cas9-HR 3 and Cas9. Specific staining is seen for Cas9-HR 3 at both full length as well as for various lower molecular weight fragments, indicating these are degradation fragments from full length Cas9-HR3, and not from other protein contaminates. Finally, similar size shift as in human cells can be seen between full length Cas9-HR 3 and Cas9 (black arrows top and bottom).

FIGS. 8A-C illustrate genomic integration with Cas9-HR shows no detectable biases compared to Cas9. FIG. 8A. Top: Diagram showing the H2B-mNeon RT; 5′ primers; and 3′ primers. Bottom left, right: Agarose gel images showing successful amplification of 5′ and 3′ PCR productions from gDNA isolated from K562 cells transfected with Cas9-HR 4, 8, Cas9 and H2B-mNeon RT, but not from untransfected control cells (Con). FIG. 8B. Sanger sequencing traces from gel purified 5′ and 3′ products from Cas9-HR 8. Both show no gross sequence abnormalities, bars: silent mutations added in the guide sequence, arrows: putative genomic SNPs, Black bars: genomic sequences, Medium gray: hH2Bb CDS, and light gray: mNeon CDS. FIG. 8B discloses SEQ ID NOS 224-227, respectively, in order of appearance. FIG. 8C. Sequence consensus alignments from sequenced PCR products form Cas9-HRs 4,8 and Cas9 when aligned to the putative integrated repair template and genomic sequences. Strong consensus is seen for all, indicating likely no gross repair differences exist between Cas9-HRs and Cas9. FIG. 8C discloses SEQ ID NOS 228-235, respectively, in order of appearance.

FIGS. 9A-D illustrate purified Cas9-HR 3 cleavage analysis. FIG. 9A illustrates SDS-PAGE Gel comparing Cas9-HR 3 vs Cas9 and reducing vs non-reducing conditions. 3-8% Tris-Acetate SDS PAGE gel comparing Cas9-HR vs Cas9 in either reducing or non-reducing conditions. Both full length and putatively cleaved Cas9-HR 3 run larger than Cas9. Additionally, apparent migration size is not dependent on oxidation state. Finally, only Cas9-HR 3 shows the full-length band at ˜200 kD. FIG. 9B. illustrates SDS-PAGE gel with concentrated Cas9-HR 3. SDS-PAGE gel with a larger amount of Cas9-HR 3 loaded. The band marked with the arrow likely corresponds to full length Cas9-HR 3, and additionally demonstrates the faint band seen in other gels is not an artifact. FIG. 9C. Western blot of purified Cas9-HR 3 and Cas9. Western blot against Cas9 on either Cas9-HR 3 or Cas9 (middle lane left blank; staining is likely overflow from Cas9-HR3). Western blot shows expected ˜40 kD size shift from full length Cas9-HR 3 (top black arrow) and Cas9 (bottom black arrow). Additionally, detection of the lower molecular weighs through western blot indicates that they are simply degraded fragments of Cas9-HR3, and not contamination by other proteins. FIG. 9D illustrates Pifithrin-α dilution series in A549 cells. A549 cells were plated in 96 well plates, transfected with Cas9 (NT) targeting the intergenic region on Chromosome 6, and treated with either DMSO or increasing concentrations of Pifithrin-α. Dose dependent increases in cellular viability can be seen with increasing concentrations of Pifithrin-α, with 10 μM showing a statistically significant (p<0.001) increase in viability compared to DMSO or Cas9 only treated cells (students t-test, two-sided).

FIG. 10A illustrates RLucRT HDR assay with A549 cells transfected with Cas9-HR 8 and Cas9 G1. Graph shows normalized luminescence levels for Cas9-HR 8 vs Cas9. As with H1299 cells, Cas9-HR 8 shows significantly higher HDR rates (˜2.5×) compared to Cas9.

FIG. 10B illustrates effect of Pifithrin-α in A549 cells transfected with either Cas9-HR 8 or Cas9. Graph demonstrates increase in cellular viability for cells transfected with Cas9 and treated with 10 μM Pifithrin-α compared to DMSO, whereas treatment of A549 cells transfected with Cas9-HR 8 with 10 μM Pifithrin-α has no effect compared to DMSO treatment. Pifithrin-α Cas9-HR 8 and Cas9 were normalized to DMSO treated Cas9-HR 8 and Cas9 respectively in order to facilitate comparison. This is further evidence that the increase in viability with Cas9-HR 8 likely comes from non-activation of the p53 pathway.

FIG. 10C illustrates effect of Pifithrin-α in A549 cells transfected with either Cas9-HR 8 or Cas9. Non DMSO treatment normalized comparison of Pifithrin-α and DMSO treated A549 cells transfected with Cas9-HR8 and Cas9.

FIG. 11A illustrates an exemplary Beta-Catenin1:mCherry RT design diagram showing the human genomic region surrounding the last exon (exon 16) of Beta-Catenin1. Three different gRNAs are denoted by black arrows (G1, G2, G3), with arrow direction indicating the targeted strand. A repair template was constructed containing approximately 750 bp long 5′ and 3′ homology arms, exon 16 of Beta-Catenin1, silent mutations introduced to prevent gRNA binding to the RT, and a direct fusion of the fluorescent protein mCherry.

FIG. 11B illustrates quantification of Beta-Catenin1:mCherry HDR rate in H1299 cells showing the average mCherry+ cells per image section (>10 for all conditions) imaged at day 3 post transfection. Both Cas9-HR8 and Cas9 showed significant increases in positive cell count compared to Beta-Catenin1:mCherry alone. Compared to Cas9, Cas9-HR8 showed significant increases in mCherry+ cells compared to Cas9 for all three gRNAs. When comparing gRNA performance within Cas9-HR8 or Cas9 treated cells, G3 showed the highest HDR efficiency and similar patterns of increases for both Cas9-HR8 and Cas9, indicating Cas9-HR8 augmented HDR efficiency, rather than fundamentally changing the behavior of Cas9.

FIG. 11C illustrates relative fold increase in Beta-Catenin1:mCherry HDR (Cas9-HR8 normalized to Cas9) showing the normalized fold change of Cas9-HR compared the corresponding guide polynucleotide for Cas9 (e.g., Cas9-HR8 G1/Cas9 G1). All guide polynucleotides showed significant (>2 fold) increases in mCherry+ cells relative to Cas9, with G2 and G3 showing the highest (˜2.5) fold change.

FIG. 11D illustrates representative images of Beta-Catenin1:mCherry+ Cells showing images from Bright Field (BF), mCherry (for increased visibility of cells) and a merge of the two for Cas9-HR8 and Cas9 G3, and RT only. Beta-Catenin1 is primarily localized to the membrane, however, can localize to the nucleus upon Wnt pathway activation. The expected membrane localization of mCherry (and possible nuclear localization in two cells of Cas9-HR8 G3) was seen in all mCherry+ cells regardless of condition, though as quantified, Cas9-HR8 had a significantly higher amount of mCherry+ cells compared to Cas9 or RT.

FIG. 11E illustrates an exemplary construct containing the mCherry targeted by two sets of primers and an exemplary agarose gel showing the presence of the intended nucleic acid fragments.

FIG. 12A illustrates a graph quantifying normalized Beta-Catenin1:mCherry Knock-in rates in HEK293 cells transfected with B-Cat-G3, Beta-Catenin1:mCherry repair template (RT) and either Cas9-HR8 or Cas9-NT (no tag). Cas9-HR8 shows significant increases in HDR rates (˜2.5×) compared to Cas9. Two replicates were performed per treatment. (P<0.01 two-sided t-test). Demonstrates Cas9-HR8 HDR improvement in an additional cell type.

FIG. 12B illustrates a graph quantifying absolute Beta-Catenin1:mCherry+ cells in HEK293 cells transfected with Beta-Catenin-G1 and Beta-Catenin1:mCherry RT and either Cas9-HR8, Cas9, or Beta-Catenin1:mCherry repair template alone. Cas9-HR8 shows significant increases in mCherry+ cells relative to Cas9-NT or repair template alone (P<0.01 two-sided t-test Cas9-HR8 vs NT or RT). Both Cas9-HR8 and Cas9 show significantly increased numbers of mCherry+ cells compared to repair template (p<0.001 for Cas9-HR8 and NT vs RT). Demonstrates Cas9-HR8 HDR improvement in an additional cell type.

FIG. 12C illustrates an inverted grayscale example image of Beta-Catenin1:mCherry knock-ins in HEK293 cells transfected with B-Cat-G3 and Cas9-HR8 and Beta-Catenin1:mCherry RT. Dark black dots represent mCherry+ HEK293 cells.

FIG. 12D illustrates an inverted grayscale example image of Beta-Catenin1:mCherry knock-ins in HEK293 cells transfected with B-Cat-G3 and Cas9-NT and Beta-Catenin1:mCherry RT. Dark black dots represent mCherry+ HEK293 cells.

FIG. 12E illustrates an inverted grayscale example image of Beta-Catenin1:mCherry knock-ins in HEK293 cells transfected with Beta-Catenin1:mCherry RT. Dark black dots represent mCherry+ HEK293 cells.

FIG. 13A illustrates a Cadherin1:mCherry RT design showing the human genomic region surrounding the last exon (exon 16) of Cadherin1. Two different gRNAs are denoted by black arrows (G1, G2), with arrow direction indicating the targeted strand. A repair template was constructed containing about 750 bp long 5′ and 3′ Homology arms, exon 16 of Cadherin1, silent mutations introduced to prevent gRNA binding to the RT, and a direct fusion of the fluorescent protein mCherry.

FIG. 13B illustrates quantification of Cadherin1:mCherry HDR rate in H1299 cells showing the average mCherry+ cells per image section (>10 for all conditions) imaged at day 3 post transfection. Both Cas9-HR8 and Cas9 showed significant increases in positive cell count compared to Cadherin1:mCherry alone. Compared to Cas9, Cas9-HR8 showed significant increases in mCherry+ cells compared to Cas9 for all three gRNAs. When comparing gRNA performance within Cas9-HR8 or Cas9 treated cells, G1 showed the highest HDR efficiency and similar patterns of increases for both Cas9-HR8 and Cas9, again further indicating Cas9-HR8 simply augments HDR efficiency, rather than fundamentally changing the behavior of Cas9.

FIG. 13C illustrates graph showing relative fold increase in Cadherin1:mCherry HDR (Cas9-HR8 normalized to Cas9) the normalized fold change of Cas9-HR compared the corresponding guide polynucleotides for Cas9 (e.g. Cas9-HR8 G1/Cas9 G1). All guide polynucleotides showed significant (>1.5 fold) increases in mCherry+ cells relative to Cas9, with G1 showing the highest (˜2.3) fold change.

FIG. 13D illustrates representative images of Cadherin1:mCherry+ Cells showing representative images from Bright Field (BF), mCherry (increased visibility of cells) and a merge of the two for Cas9-HR8 and Cas9 G1, and RT only. Cadherin1 is only localized to the membrane, and the expected membrane localization of mCherry (and possible nuclear localization in two cells of Cas9-HR8 G3) was seen in all mCherry+ cells regardless of condition. Additionally, as quantified, Cas9-HR8 had a significantly higher amount of mCherry+ cells compared to Cas9 or RT.

FIG. 13E illustrates an exemplary construct containing the mCherry targeted by two sets of primers and an exemplary agarose gel showing the presence of the intended nucleic acid fragments.

FIG. 14A illustrates quantifying of normalized CDH1:mCherry Knock-in rates in HEK293 cells transfected with Ecad-G1, CHD1:mCherry repair template (RT) and either Cas9-HR8 or Cas9-NT (no tag). Cas9-HR8 shows significant increases in HDR rates (˜3.5×) compared to Cas9. Two replicates were performed per treatment. (P<0.0001 two-sided t-test for all) Demonstrates Cas9-HR8 HDR improvement in an additional cell type.

FIG. 14B illustrates quantifying of absolute CDH1:mCherry+ cells in HEK293 cells transfected with Ecad-G1 and CHD1:mCherry RT and either Cas9-HR8, Cas9, or CDH1:mCherry repair template alone. Cas9-HR8 shows significant increases in mCherry+ cells relative to Cas9-NT or repair template alone (P<0.001 two-sided t-test Cas9-HR8 vs NT or RT). Both Cas9-HR8 and Cas9 show significantly increased numbers of mCherry+ cells compared to repair template (p<0.0001 for Cas9-HR8 and NT vs RT). Demonstrates Cas9-HR8 HDR improvement in an additional cell type.

FIG. 14C illustrates an inverted grayscale example image of CDH1:mCherry knock-ins in HEK293 cells transfected with Ecad-G1 and Cas9-HR8 and CDH1:mCherry RT. Dark black dots represent mCherry+ HEK293 cells.

FIG. 14D illustrates an inverted grayscale example image of CDH1:mCherry knock-ins in HEK293 cells using Ecad-G1 and Cas9-NT. Dark black dots represent mCherry+ HEK293 cells.

FIG. 14E illustrates an inverted grayscale example image of CDH1:mCherry knock-ins in HEK293 cells using RT only. Dark black dots represent mCherry+ HEK293 cells.

FIG. 15A illustrates whole well imaging of Cas9-HR8 HDR rates of Cadherin1:mCherry genomic integration. Stitched Images from 60 independent sections were taken from cells in a 12 well plate of H1299 cells 4 Days after transfection with Cas9-HR8, Cadherin1:mCherry RT and Ecad-G1 with Brightfield (BF), mCherry, and merged images. Lower images showed enlarged sections (dashed box) as examples of HDR rates. Though high background prevented absolute quantification, Cas9-HR8 showed significant amounts of mCherry+ cells.

FIG. 15B illustrates whole well imaging of Cas9 HDR rates of Cadherin1:mCherry genomic integration. Stitched Images from 60 independent sections were taken from cells in a 12 well plate of H1299 cells 4 Days after transfection with Cas9, Cadherin1:mCherry RT and Ecad-G1 with Brightfield (BF), mCherry, and merged images. Lower images showed enlarged sections (dashed box) as examples of HDR rates. Though high background prevented absolute quantification, Cas9 showed low, though detectable amounts of mCherry+ cells.

FIG. 15C illustrates whole well imaging of HDR rates of Cadherin1:mCherry genomic integration. Stitched Images from 60 independent sections were taken from cells in a 12 well plate of H1299 cells 4 Days after transfection with Cadherin1:mCherry RT with Brightfield (BF), mCherry, and merged images. Lower images show enlarged sections (dashed box) as examples of HDR rates. Though high background prevented absolute quantification, Cadherin1:mCherry RT alone showed very low rates of mCherry+ cells.

FIG. 15D illustrates combined sections of whole well imaging of HDR rates of Cadherin1:mCherry genomic integration. Combined image sections were obtained from cells transfected with either Cas9-HR8 or Cas9, Ecad-G1, Cadherin1:mCherry RT or Cadherin1:mCherry RT alone, shown with Brightfield (BF), mCherry, and merged images. Though high background prevented absolute quantification, Cas9-HR8 shows significantly higher amounts of mCherry+ cells relative to Cas9 or RT alone (Cas9-HR>Cas9>>RT).

FIG. 16A illustrates HDR rates of fusions of Dna2(1-397)-AP5X-Cas9 and Dna2 (1-397)-Cas9, compared to Cas9-HR8, Cas9 or Cadherin1:mCherry RT alone showing the average mCherry+ cells per image section (>10 for all conditions) imaged at day 3 post transfection. All of Dna2 (1-397)-AP5X-Cas9, Dna2 (1-397)-Cas9, Cas9-HR8 and Cas9 showed significant increases in mCherry+ cell count compared to Cadherin1:mCherry RT alone. Compared to Cas9, Cas9-HR8 showed significant increases in mCherry+ cells, whereas Dna2 (1-397)-AP5X-Cas9 and Dna2(1-397)-Cas9 showed roughly similar levels of mCherry+ cells.

FIG. 16B illustrates normalized Cadherin1:mCherry HDR rates of Dna2 (1-397) and Cas9-HR to Cas9 showing the normalized fold change of Dna2 (1-397)-AP5X-Cas9, Dna2 (1-397)-Cas9, and Cas9-HR8 compared to Cas9. Cas9-HR8 had a significantly higher HDR rate than Dna2 (1-397)-AP5X-Cas9, Dna2 (1-397)-Cas9, or Cas9, thereby demonstrating that fusion of the 5′->3′ exonuclease domain of Dna2(1-397) either through a stiff AP5X linker or directly was not sufficient to increase HDR rates.

FIG. 17A illustrates that Cas9-HR reduced cellular toxicity in A549 cells compared to Cas9 when targeted to SHS-231. Quantification of cellular viability in A549 cells transfected with Cas9-HR8 or Cas9 targeting either SHS-231 G1, G2, or G3 was measured on Day 2 post transfection via rezarufin/rezazurin metabolic activity assay. Cas9-HR8 showed significant increases in cellular viability relative to Cas9, with G2 and G3 showing greatest increases in cellular viability.

FIG. 17B illustrates that Cas9-HR reduced cellular toxicity in A549 cells compared to Cas9 when targeted to Cadherin1. Quantification of cellular viability in A549 cells transfected with Cas9-HR8 or Cas9 targeting either Cadherin1 G1 or G2 was measured on Day 2 post transfection via rezarufin/rezazurin metabolic activity assay. Cas9-HR8 showed significant increases in cellular viability relative to Cas9, with G1 showing the greatest increase in cellular viability, as expected based on pervious correlation of reduction of toxicity and increase in HDR rate.

FIG. 17C illustrates that Cas9-HR significantly increases HDR rates for an mNeon expression cassette at a previously identified Safe Harbor Site (SHS)-231. Stitched Images from 78 independent sections were obtained from a 6 well plate of H1299 14 Days after transfection with Cas9-HR8 or Cas9, SHS-231-pCAG-mNeon-bGHPa-RT and SHS-231 G1, with Brightfield (BF), GFP, and merged images. Cas9-HR8 and Cas9 GFP intensities had been increased to aid visualization of GFP+ cells. Cas9-HR showed both significantly more GFP+ cells, as well as significantly more total cells compared to Cas9.

FIG. 18A illustrates SHS-231-pCAG-mNeon-bGHPa RT design showing the human genomic region surrounding SHS-231 (Chr4:58974613-58978632). Three different gRNAs are denoted by black arrows (G1, G2, and G3), with arrow direction indicating the targeted strand. A repair template was constructed containing about 900 bp long 5′ and 3′ homology arms, pCAG (a synthetic strong constitutive promoter), mNeon, and a bGH poly A site (bGHPa). Silent mutations introduced to prevent gRNA binding to the RT are shown in red.

FIG. 18B illustrates box plots showing cellular fluorescence levels quantified for mNeon+ cells from either Cas9-HR8 or Cas9 treated cells 14 days transfection. Cas9-HR cells not only showed significantly more mNeon+ cells, but also showed much more uniform and lower expression levels (significantly reduced sizes of quartile ranges and average) compared to Cas9. This is indicative of vastly increased single site, stable integration of SHS-mNeon transgenes relative to Cas9, as cells with single stable integrations would be expected to have significantly lower fluorescence levels than multiple or other improper integration events.

FIG. 19A illustrates in vitro transcription of 5′ Capped and Poly-A tailed Cas9-HR8 mRNA. Cas9-HR8 (with Cas9 as a reference) was in vitro transcribed from a template containing a T7 promoter, strong Kozac initiation sequence, Cas9-HR8 CDS and a about 150 bp poly-A tail. Reactions were run on a 1% TAE gel for about 1 hr, a strong band at ˜2 kb was present in both Cas9-HR8 lanes, indicating transcription of full length Cas9-HR (as expected on a native gel, Cas9 ran at ˜1.8 kb). Additionally, lanes were also run with an extra micro liter of GTP added as GTP could be rate limiting for longer constructs, as shown indicated on the gel.

FIG. 19B illustrates that Cas9-HR8 editing produced roughly 10× mNeon+ cells relative to Cas9 at 14 days post-transfection. Quantification of the number of mNeon+ cells in either Cas9-HR8 or Cas9, SHS-231-G1, SHS-231-mNeon RT treated H1299 cells 14 days post-transfection was obtained. FIJI (ImageJ) software was used to identify and quantify mNeon+ cells for both Cas9-HR8 and Cas9, with Cas9-HR8 showing a huge increase (3652 vs 359, >10×) in absolute numbers of mNeon+ cells relative to Cas9.

FIG. 20A illustrates a graph showing total cell counts from two independent experiments transfecting H1299 cells with either Cas9-HR8 or Cas9, SHS-231-G2 and an mNeon transgene. Experiment differs from previous exp due to non-catastrophic death of Cas9 treated cells following trypsinization, proving that increase seen with Cas9-HR8 was not due simply to cell death, but also due to increase of HDR rates.

FIG. 20B illustrates a graph showing normalized cell counts to Cas9 from two independent experiments transfecting H1299 cells with either Cas9-HR8 (red) or Cas9 (NT), SHS-231-G2 and an mNeon transgene (p<0.00001 Cas9-HR8 vs Cas9). Experiment differs from previous exp due to non-catastrophic death of Cas9 treated cells following trypsinization, proving that increase seen with Cas9-HR8 was not due simply to cell death, but also due to increase of HDR rates.

FIG. 20C illustrates an inverted grayscale example image of SHS-231-pCAG-mNeon knock-ins in H1299 cells transfected with SHS-G2 and Cas9-HR8 and SHS-231-pCAG-mNeon RT. Dark black dots represent mNeon+H1299 cells.

FIG. 20D illustrates an inverted grayscale example image of SHS-231-pCAG-mNeon knock-ins in H1299 cells transfected with SHS-G2, Cas9-NT and SHS-231-pCAG-mNeon RT. Dark black dots represent mNeon+H1299 cells.

FIGS. 21A-D illustrate a diagram of experiments to quantify replication protein A (RPA) foci in Cas9-HR8 or Cas9 treated U2OS cells and confocal microscopy images of the transfected U2OS cells. U2OS cells were transfected with Cas9-HR8 or Cas9 targeting either SHS-231, hH2B-G4 or B-Catenin1, or non-treated control cells. After two days cells were fixed, then cytoplasm extracted on ice, then remaining nuclei were stained for RPA. At the end of staining, nuclei were labeled with DAPI and cells were imaged via confocal microscopy. Post-imaging nuclei identification and RPA thresholding allowed quantification of the number of RPA foci per nuclei. FIGS. 21B-D illustrate exemplary confocal images of RPA foci stained U2OS cells transfected with Cas9 (complexed with guide RNA 4 targeting hH2B, FIG. 21B); Cas9-HR8 (complexed with guide RNA 4 targeting hH2B, FIG. 21C); and U2OS control cells (FIG. 21D). FIG. 21E illustrates a graph of percent cells above with any RPA foci in U2OS cells transfected with Cas9-HR8 or Cas9 targeting either SHS-231, hH2B-G4 or B-Catenin1, or non-treated control cells. Both Cas9-HR8 and Cas9 increase the percentage of cells with RPA foci, though Cas9 shows a greater increase relative to Cas9-HR8. FIG. 21F illustrates a graph of percent cells with 1-10 RPA foci in U2OS cells transfected with Cas9-HR8 or Cas9 targeting either SHS-231, hH2B-G4 or B-Catenin1, or non-treated control cells. Again, both Cas9-HR8 and Cas9 increase the percentage of cells with RPA foci, though Cas9 shows a greater increase relative to Cas9-HR8, particularly in hH2B and B-Catenin1 treated cells. FIG. 21G illustrates a graph of percent cells with 11-100 RPA foci in U2OS cells transfected with Cas9-HR8 or Cas9 targeting either SHS-231, hH2B-G4 or B-Catenin1, or non-treated control cells. Here, Cas9-HR8 shows a significant decrease in cells with 11-100 RPA foci compared to Cas9 targeting both hH2B and Beta-Catenin1, demonstrating that Cas9-HR8 can significantly decrease genomic stress (as shown by large amounts of RPA foci) at two independent loci compared to Cas9.

FIG. 22A illustrates a diagram of CHO cell Cas9-HR8 stable knock-in protocol. Briefly, CHO cells are transfected with Cas9-HR8, CHO-SHS-1.2-G1, and a Cas9-HR8 repair template consisting of: CHO-SHS-1.2-homology arms, pCAG (a strong constitutive promoter), Cas9-HR8, an IRES sequence, a Puromycin resistance gene, and a BGH poly adenylation signal. After recovering from electroporation, cells were treated with Puromycin for 10 days, after which they were grown an additional 12 days without puromycin. A portion of cells were lysed while others were frozen, and either control or Cas9-HR8 knock-in CHO cells were probed for expression via anti-Cas9 western blot.

FIG. 22B illustrates a SDS-PAGE showing strong staining of, which is the predicted size for Cas9-HR8, in Cas9-HR8 CHO cells thereby demonstrating that the Cas9-HR8 CHO cell line has stably integrated Cas9-HR8, and expression should remain stable for long-term growth. Purified recombinant Cas9 is included as a sizing comparison.

FIG. 23 illustrates two exemplary constructs for utilizing the embodiments described herein.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

CRISPR/Cas9 has revolutionized genetic engineering, however, the inability to control double strand break (DSB) repair has severely limited both therapeutic and academic applications. Many attempts have been made to control DSB repair choice, however particularly in the case of larger edits, none have been able to bypass the rate-limiting step of Homologous Recombination (HR): long-range 5′ end resection. Described herein is a novel set of Cas9 fusions, Cas9-HRs, designed to bypass the rate-limiting step of HR repair by simultaneously coupling initial and long-range end resection, and demonstrate that Cas9-HRs can increase the rate of Homology Directed Repair (HDR) by 2-2.5 fold and decrease p53 mediated cellular toxicity by 2-4 fold compared to Cas9. Cas9-HRs are functional in multiple mammalian cell lines with minimal apparent editing site bias, thus making Cas9-HRs an attractive option for applications demanding increased HDR rates for long inserts and/or reduced p53 pathway activation.

CRIPSR/Cas9 generally has two broad uses in genome editing: mutation of targeted sites via imprecise Non-Homologous End Joining (NHEJ) mediated repair and introducing precise edits in the genome ranging from 1 to 1000s of base-pairs (bp) using an external template sequence via Homology Directed Repair (HDR)4. While HDR methods generally allow for more flexible editing, NHEJ repair is highly preferred in higher eukaryotes where HDR repair rates can be as much as two orders of magnitude less than NHEJ repair. Low HDR/INDEL ratios have so far generally limited therapeutic applications of CRISPR/Cas9 to only those which are amenable to NHEJ based repair, and makes the introduction of targeted mutations, insertions or deletions difficult, expensive and time consuming. In addition, NHEJ based repair can activate the p53 pathway, prolonged activation of which can lead to cellular apoptosis, not only reducing yields of edited cells but also potentially selecting for cancerous mutations.

There are many different approaches that have been taken to attempt to improve HDR rates, however most fall into four broad classes: small molecule and genetically encoded inhibitors or activators of NHEJ and HDR respectively, cell cycle synchronization, optimization of culture conditions, and engineering of the nuclease(s) themselves. For example, inhibiting DNA ligase IV, a key step in the NHEJ repair pathway, or stimulating Cyclin D1, which governs the transition from G0 to G1/S phase, can increase HDR 2-3 fold. While these can be effective in-vitro, these techniques must be optimized for each cell type, and can have undesirable side effects which significantly limit their usefulness. Significant effort has been put into engineering Cas9 (and other nucleases) to provide more flexible alternatives, with some of the most successful being base-editors and prime-editing, both of which demonstrated significant (>20%) error free editing for both single-base (A->G and C->T) and short (˜<40 bp) insertions or deletions with minimal cellular toxicity in multiple cell types. While there are still some issues to resolve with these tools, particularly off-target effects, there is no doubt that they have dramatically impacted the landscape of genetic engineering and point to the significant benefits that engineered Cas9 fusions can bring.

Unfortunately, Cas9 fusions designed to increase the HDR rate for longer inserts (>0.3 kb) have not been as successful, though progress has been made by some groups. Recently, It has been shown that fusion of various factors involved in double strand break (DSB) repair choice (CtIP, Mre11, and a truncated piece of p53 named DN1s) to Cas9 can increase the ratio of HDR/INDEL repair for longer inserts from ˜1.5-2.5 fold, depending on fusion partner. While these results demonstrate this type of approach can succeed, each of these fusions still has significant issues. Cas9-CtIP has been shown to have both editing site and cell type bias, limiting their potential usefulness significantly. Additionally, given that both CtIP and Mre11 undergo extensive post-translational regulation and have a myriad of protein:protein interactions, it is likely that these issues are related to endogenous cellular components themselves, and if so, unlikely to be improved upon. Additionally, none of these fusions have directly been shown to reduce cellular toxicity, in fact the most effective in terms of HDR/INDEL ratio increase, Cas9-DN1s, actually increases toxicity by roughly 5-10%16. Equally important, their mechanisms of action fundamentally limit their effectiveness to endogenous rates of HDR repair, as none of these fusions act on what is thought to be the rate limiting step: long range 5′->3′ end resection.

Eukaryotic DSB repair takes place via two main pathways: canonical and non-canonical. The first relies on binding of MRN/CtIP complex slightly downstream of the DSB, which then resect back via 3′->5′ exonuclease activity to create short (<20 bp) single strand (ss) DNA 3′ ends. After additional steps, the overhanging ssDNA 3′ ends are eventually further resected via long range 5′->3′ exonucleases Exo1 or Dna2, which then commits the DSB to be repaired via HR17. The non-canonical pathway simply by-passes the initial 3′->5′ resection, with either Exo1 or Dna2 directly initiating and resecting the DSB 5′ ends to create long 3′ ssDNA ends, thus committing the DSB to HR repair. It is thought that the canonical repair pathway prevails due to DBS ends being “blocked” by other bound proteins20, however, fusion of either Exo1 or Dna2 to Cas9 should allow them preferential access to the DSB, in theory greatly increasing the chance of committing the DSBs to HR via the non-canonical repair pathway.

While both human Exo1 and Dna2 were considered as initial fusion partners, ultimately, hExo1 was chosen as due to the greater amounts of biochemical and structural data available compared to Dna2. Full length hExo1 is a relatively large protein at 846AA in length that can be divided into roughly two regions: the N-terminal exonuclease region (1-392), and the C-terminal region that interacts with MLH2/MSH1 DNA mismatch repair proteins (393-846). While Exo1 activity and stability is also extensively post-translationally regulated by phosphorylation (similar to CtIP, Mre11 and Dna2), it has several key differences: Exo1 functions as a monomer and all but one of the putative post-translational regulatory phosphorylation sites lie in the C-terminal region, deletion of which does not impede exonuclease activity. Finally, the Escherichia coli (E. coli) homologue of Exo1, which possesses 3′->5′ exonuclease activity as opposed to 5′->3′ for hExo1, was fused to Cas9 and was shown to generate much longer deletions than traditional Cas9, indicating that successful fusion of hExo1 may also be possible27. Therefore, various hExo1-Cas9 fusions were designed to test whether directing cells to the HR repair pathway via the non-canonical pathway can increase HDR rates.

The ability to control DSB repair pathway choice has been long sought after, with numerous different tools and techniques developed to influence repair choice. As described here, Cas9-HRs represent the first ever example of a tool designed to act at the rate limiting step of HR repair. As a first pass for the efficacy of this tool type, HDR assays for Cas9-HRs (particularly Cas9-HR 8) have demonstrated significant increases in HDR rates (˜2-2.8 fold) compared to Cas9 at multiple loci, across multiple cell and assay types. Interestingly, in addition to increased HDR rates, Cas9-HRs also show significantly reduced activation of the p53 pathway, potentially allowing for extension of high efficiency HDR methods to more sensitive cell types, or even select in-vivo applications. It will be particularly interesting to investigate the exact mechanisms behind Cas9-HRs ability to increase HDR rates and reduce cellular toxicity, as this potentially could shed further light on fundamental principles governing eukaryotic DSB repair choice.

Given other experiences with Cas9 fusion proteins, it is likely that further rounds of engineering will be required to improve Cas9-HR efficacy and hopefully boost HDR rates even further. Additionally, since the current size of Cas9-HRs (˜5.5 kb) makes them slightly too large for current adenovirus techniques, fusions using minimal Cas9s or other more compact RNA guided nucleases can be developed, in addition to other fusions and mutations designed to expand Cas9-HR functionality and targetability.

Bacterial expression and purification of Cas9-HRs appears to be viable, though significant optimization of expression conditions or further engineering of Cas9-HR will likely be required before in-vivo testing of Cas9-HR RNP performance is possible. Nevertheless, even in their current state the Cas9-HR platform represents a significant step forward in controlling DSB repair choice and should prove particularly useful for applications demanding increased HDR rates for long inserts and/or reduced p53 pathway activation.

Described herein is a fusion protein complex for increasing HDR rates through bypassing the rate limiting step of homologous recombination repair. In some cases, fusion protein can be complexed with at least one guide polynucleotide described herein to form a fusion protein complex. In some instances, the fusion protein described herein is a Cas fusion protein (e.g., any one of the Cas9-HR described herein) that can introduce a polynucleotide of interest into a genomic locus. In some cases, the Cas fusion protein can introduce a polynucleotide of interest that is longer than compared to a comparable polynucleotide of interest that can be introduced by a conventional Cas proteins. In some cases, the polynucleotide of interest can be a repair template (e.g., encoding nucleic acid sequence that corrects mutation in the genomic locus). In some embodiments, the polynucleotide of interest can be a transgene. In some embodiments, the polynucleotide of interest can be a regulatory element that regulates gene expression in a cell. In some instances, the Cas fusion protein complex can lead to decreased endogenous p53 signaling or decreased cellular toxicity compared to conventional Cas protein.

Also described herein are methods utilizing any one of the fusion protein or fusion protein complex described herein for treating a disease or a condition. In some embodiments, the disease or the condition is cancer. In some embodiments, the cancer is caused by the presence of endogenous genetic mutations. For example, the cancer can be caused by endogenous genetic mutation in Catenin or Cadherin. In some embodiments, the cancer can be treated by repairing the endogenous genetic mutations. In some embodiments, the cancer can be treated by replacing a fragment of the endogenous gene comprising the genetic mutation with a polynucleotide of interest (e.g., a repair template or a HDR template) described herein. In some embodiments, a polynucleotide of interest is introduced into an endogenous genomic locus to replace the endogenous gene fragment containing the genetic mutation via the HDR induced by the any one of the fusion protein or fusion protein complex described herein. In some embodiments, the method comprises utilizing Cas fusion protein or Cas fusion protein complex for treating the disease or condition.

### Fusion Protein

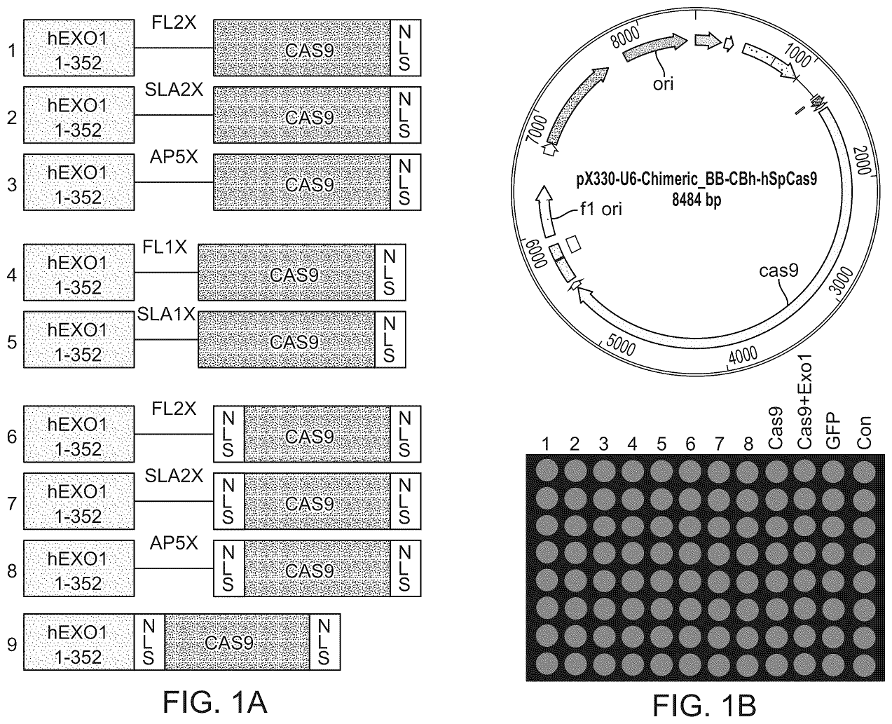

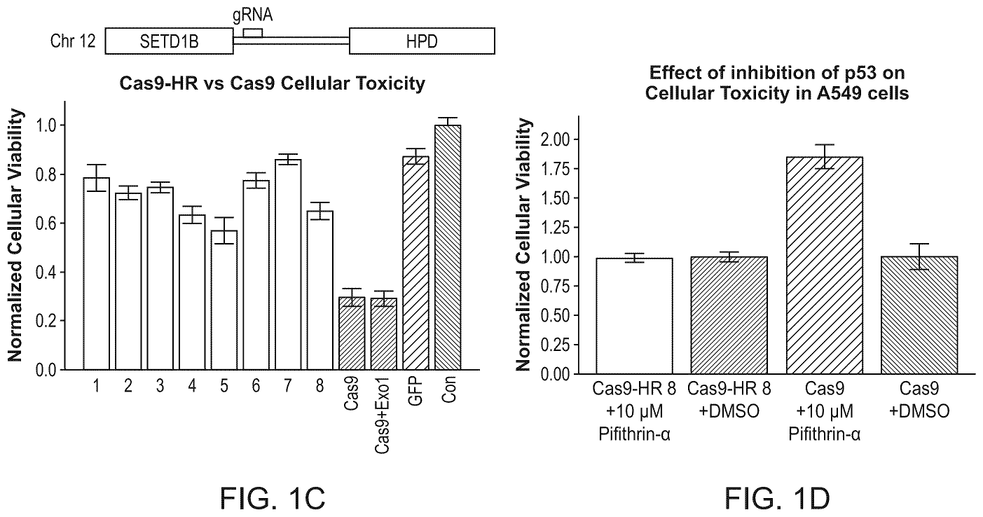

Described herein are fusion proteins, where a programmable endonuclease is fused to at least one additional exonuclease. In some embodiments, the programmable endonuclease is a Cas protein. In some embodiments, the programmable endonuclease and the exonuclease are connected via a peptide linker. FIG. 1A illustrates exemplary arraignments of fusion protein (Cas9-HR) described herein. In some embodiments, the programmable endonuclease comprises a programmable Cas such as Cas9. As used herein, the “Cas9,” “Cas9 domain,” or “Cas9 fragment” refers to an RNA-guided nuclease comprising a Cas9 protein, or a fragment thereof, e.g., a protein comprising an active DNA cleavage domain of Cas9. A Cas9 nuclease is sometimes referred to as a casn1 nuclease or a CRISPR (clustered regularly interspaced short palindromic repeat)-associated nuclease. Cas9 nuclease sequences and structures are well known to those of ordinary skill in the art. Cas9 orthologs have been described in various species, including, but not limited to, S. pyogenes and S. thermophilus. Wild type (unmodified) Cas9 can be from any of the sequences encoded from SEQ ID NOs: 7-23 or SEQ ID NOs: 41-54.

In some embodiments, the programmable Cas can include Class 1 Cas polypeptides, Class 2 Cas polypeptides, type I Cas polypeptides, type II Cas polypeptides, type III Cas polypeptides, type IV Cas polypeptides, type V Cas polypeptides, and type VI CRISPR-associated (Cas) polypeptides, CRISPR-associated RNA binding proteins, or a functional fragment thereof. Further, Cas polypeptides suitable for use with the present disclosure often include Cpf1 (or Cas12a), c2c1, C2c2 (or Cas13a), Cas13, Cas13a, Cas13b, c2c3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Csn1, Csx12, Cas10, Cas10d, Cas1O, Cas1Od, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and Cu1966; any derivative thereof, any variant thereof, and any fragment thereof. In some embodiments, the programmable Cas is Cas12 such as Cas12a, Cas12b, Cas12c, Cas12d, Cas12e, Cas12f, or Cas12j. In some embodiments, the programmable Cas is Cas12a. Exemplary programmable Cas12 can be encoded from any of the sequences SEQ ID NOs: 55-57.

In some embodiments, the programmable Cas can be substituted with another programmable endonuclease. For example, other site-specific endonucleases that are suitable for the fusion protein composition disclosed herein often comprise zinc finger nucleases (ZFN); transcription activator-like effector nucleases (TALEN); meganucleases; RNA-binding proteins (RBP); recombinases; flippases; transposases; Argonaute (Ago) proteins (e.g., prokaryotic Argonaute (pAgo), archaeal Argonaute (aAgo), and eukaryotic Argonaute (eAgo)); or any functional fragment thereof.

The Cas9 enzymes or other programmable nuclease disclosed herein also comprises at least one nuclear localization signal (NLS), which is an amino acid sequence that attaches to a protein for import into the cell nucleus by nuclear transport. Generally, the NLS comprises one or more short sequences of positively charged lysines or arginines exposed on the protein surface. These types of classical NLSs can be further classified as either monopartite or bipartite. The major structural difference between the two is that the two basic amino acid clusters in bipartite NLSs are separated by a relatively short spacer sequence (hence bipartite—2 parts), while monopartite NLSs are not. In some embodiments, the NLS comprises sequence PKKKRKV (SEQ ID NO: 221) of the SV40 Large T-antigen (a monopartite NLS). In other embodiments, the NLS of nucleoplasmin comprises sequence KR[PAATKKAGQA]KKKK (SEQ ID NO: 222). There are also many other types of non-classical NLSs. Different types of NLSs disclosed herein are not meant to be limiting and a person of ordinary skill in the art is able to select a NLS to attach to a Cas9 protein. In some embodiments, the Cas9 protein comprises an N-terminal NLS. In other embodiments, the Cas9 protein comprises a C-terminal NLS. In yet other embodiments, the Cas9 protein comprises both N-terminal and C-terminal NLSs.

In some embodiments, the Cas fusion protein can be complexed with at least one guide polynucleotide (e.g., a gRNA) to form a fusion protein complex or a Cas fusion protein complex via a ribonucleoprotein (RNP). A RNP typically comprises at least two parts: one part comprises a programmable endonuclease such as a Cas9 or other CRISPR-related programmable endonucleases; and the other part comprises a gRNA or other specificity-conveying nucleic acid. Often, a wild type Cas9 enzyme or other Cas or non-Cas programmable endonuclease can be one part of the CRISPR-Cas9 system. The modified Cas9 protein coupled to a fragment of hExo1 via a linker peptide can also be one part of the CRISPR-Cas9 system. Further, the modified Cas9 protein and a gRNA can form a ribonucleoprotein (RNP).

In some embodiments, the Cas fusion protein (e.g., any one of the Cas9-HR) can induce an insertion of a polynucleotide of interest into a genomic locus, where the polynucleotide of interest to be inserted comprises a nucleic acid sequence that is at least 1000 nucleotides, at least 1500 nucleotides, at least 2000 nucleotides, at least 2500 nucleotides, at least 3000 nucleotides, at least 3500 nucleotides, at least 4000 nucleotides, at least 4500 nucleotides, at least 5000 nucleotides, at least 5500 nucleotides, or more nucleotides. In some embodiments, the Cas fusion protein (e.g., any one of the Cas9-HR) can induce an insertion of a polynucleotide of interest, where the length of the polynucleotide of interest, compared to a length of a polynucleotide of interest inserted by convention or wild type Cas protein, is increased by at least 500 nucleotides, at least 1000 nucleotides, at least 1500 nucleotides, at least 2000 nucleotides, at least 2500 nucleotides, at least 3000 nucleotides, at least 3500 nucleotides, at least 4000 nucleotides, at least 4500 nucleotides, at least 5000 nucleotides, at least 5500 nucleotides, or more nucleotides.

### Exonuclease

The fusion protein comprising the programmable endonuclease can be fused to an exonuclease or an exonuclease domain or fragment so as to effect the results disclosed herein. A number of exonuclease or programmable exonuclease combinations are consistent with the disclosure herein. With respect to the exonuclease, certain exemplary exonucleases suitable for use as part of the fusion protein in present application include MRE11, EXO1, EXOIII, EXOVII, EXOT, DNA2, CtIP, TREX1, TREX2, Apollo, RecE, RecJ, T5, Lexo, RecBCD, and Mungbean nuclease. Additional suitable exonucleases are also contemplated. In certain embodiments, human Exo1 (hExo1) is used herein as a part of the fusion protein. Full length hExo1 (SEQ ID NO: 1) can be divided into roughly two regions: the N-terminal nuclease region (1-392, SEQ ID NO: 2); and the C-terminal MLH2/MSH1 interaction region (393-846, SEQ ID NO: 3). In some embodiments, the N-terminal nuclease region of hExo1 (SEQ ID NO: 2) is used to covalently link to a Cas9 with at least one NLS via a peptidyl linker. In other embodiments, a fragment of SEQ ID NO: 2 or other exonuclease domain that retains the nuclease function is used herein. For example, the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to SEQ ID NO: 2. In some embodiments, the fragment may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to SEQ ID NO: 2 or other untruncated or unmutated domain. In certain embodiments, DNA replication ATP-dependent helicase/nuclease (DNA2) is used herein as a part of the fusion protein. Full length DNA2 (SEQ ID NO: 4) can be divided into roughly two regions: the N-terminal nuclease region (1-397, SEQ ID NO: 5); and the C-terminal MLH2/MSH1 interaction region (398-1060, SEQ ID NO: 6). In some embodiments, the N-terminal nuclease region of DNA2 (SEQ ID NO: 4) is used to covalently link to a Cas9 with at least one NLS via a peptidyl linker. In other embodiments, a fragment of SEQ ID NO: 4 or other exonuclease domain that retains the nuclease function is used herein. For example, the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to SEQ ID NO: 4. In some embodiments, the fragment may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to SEQ ID NO: 4 or other untruncated or unmutated domain. The N-terminal nuclease region of the hExo1 or DNA2 is exemplary, and additionally suitable Exo1 or other exonuclease sequences can be utilized for the purpose disclosed herein by a person of ordinary skill in the art.

### Construct

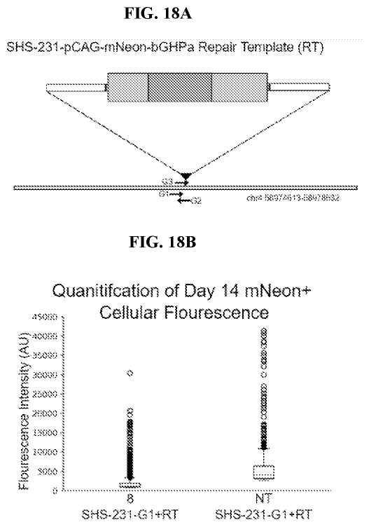

Provided herein are exemplary constructions (e.g., FIG. 18) for expressing the fusion protein (e.g. Cas9-HR), guide polynucleotide, repair template, or a combination thereof. SEQ ID NOs: 24-37 are exemplary nucleic acid sequences encoding an exemplary Cas9 described herein. SEQ ID NOs: 41-54 are exemplary polypeptide sequences (encoded from SEQ ID NOs: 24-37 respectively) of an exemplary Cas9 described herein. SEQ ID NO: 24 and SEQ ID NO: 41 encode Cas9 (D10A): Cas9 nickase mutation, where Cas9 can only cut on the target strand, which can potentially be combined with Exo1 flap nuclease activity present in Cas9-HRs to increase HDR using ssODN (single strands Oligo DNA donor), dsDNA (double strand DNA) or plasmid repair templates. SEQ ID NO: 25 and SEQ ID NO: 42 encode Cas9 (H840A): Cas9 nickase mutation, where Cas9 can only cut on the opposite strand, which can be potentially combined with Exo1 flap nuclease activity present in Cas9-HRs to increase HDR using ssODN (single strands Oligo DNA donor), dsDNA (double strand DNA) or plasmid repair templates. SEQ ID NO: 26 and SEQ ID NO: 43 encode Cas9 (SpG): Relaxation of Cas9 PAM targeting requirements (NGG->NGN, incorporation of mutations to Cas9 which results in relaxation of NGG PAM requirement to NGN) which could be incorporated into existing Cas9-HRs to increase treatable indication and editable locations in the genome. SEQ ID NO: 27 and SEQ ID NO: 44 encode Cas9 (SpRY): Relaxation of Cas9 PAM targeting requirements (NGG->NRN,NYN, incorporation of mutations to Cas9) which results in relaxation of NGG PAM requirement to NRN and some NYN and could be incorporated into existing Cas9-HRs to increase treatable indication and editable locations in the genome through Cas9-HR HDR repair. SEQ ID NO: 28 and SEQ ID NO: 45 encode Cas9 (HF): Cas9 variant designed to reduce off targeting events. These mutations could be introduced into Cas9-HR in combination with any other variants noted here in order to decrease off targeting events. SEQ ID NO: 29 and SEQ ID NO: 46 encode Cas9 (e1.1): Additional Cas9 variant designed to reduce off targeting events. These mutations could be introduced into Cas9-HR in combination with any other variants noted here in order to decrease off targeting events. SEQ ID NO: 30 and SEQ ID NO: 47 encode AsCas12a-HF1 (Cpf1-HF with relaxed PAM requirements): Cas12a engineered to have relaxed PAM requirements combined with mutations reducing off target cutting. Exo1/Dna2 of fragments of could be fused to created Cas12a-HR, which could be used in both mammalian and agricultural settings carry out HDR mediated genetic engineering; SEQ ID NO: 31 and SEQ ID NO: 48 and SEQ ID NO: 32 and SEQ ID NO: 49 encode Split Cas9 (2-573) and Split Cas9 (574-1368) respectively: Exo1/Dna2 could be fused with either fragment of Cas9 (2-573) or (574-1368), then packed in Adenoviral vectors (AVV) and delivered in mammalian cells to achieve the same benefits seen with full length Cas9-HR HDR engineering. This would allow Cas9-HRs to be used with traditional AVV techniques without significant engineering. SEQ ID NO: 33 and SEQ ID NO: 50 encode NmCas9: Alternate Cas9 for use in Cas9-HR to enable AVV techniques or use in expanded organisms; SEQ ID NO: 34 and SEQ ID NO: 51 encode SaCas9: Alternate Cas9 for use in Cas9-HR to enable AVV techniques or use in expanded organisms. SEQ ID NO: 35 and SEQ ID NO: 52 encode CjCas9: Alternate Cas9 for use in Cas9-HR to enable AVV techniques or use in expanded organisms. SEQ ID NO: 36 and SEQ ID NO: 53 encode ScCas9: Alternate Cas9 for use in Cas9-HR to enable AVV techniques or use in expanded organisms. SEQ ID NO: 37 and SEQ ID NO: 54 encode ScCas9 (++): Alternate Cas9 with increased fidelity and activity for use in Cas9-HR to enable AVV techniques or use in expanded organisms.

SEQ ID NOs: 61-69 illustrates exemplary nucleic acid sequences encoding bacterial expression vector for expressing Cas9: pET-28b (SEQ ID NO: 61): vector for lactose inducible T7 mediated expression of Cas9-HRs; pTac (SEQ ID NO: 62): vector containing combined promoter elements from Trp and Lac operons and used for lactose/IPTG inducible expression of Cas9-HRs in E. coli; pTrc (SEQ ID NO: 63): vector containing combined promoter elements from Trp and LacUV5 promoters and used for lactose/IPTG inducible expression of Cas9-HRs in E. coli; pT5 (SEQ ID NO: 64): vector containing combined promoter elements from phage T5 and lac operon promoters and used for lactose/IPTG inducible expression of Cas9-HRs in E. coli; pT7 (SEQ ID NO: 65): vector containing the strong T7 promoter and used for lactose/IPTG inducible expression of Cas9-HRs in E. coli; Cas9-HR 3 E. coli codon optimized vector (SEQ ID NO: 66): sequence of Cas9-HR 3 to be used in conjunction with any of the above vectors for bacterial expression; Cas9-HR 4 E. coli codon optimized vector (SEQ ID NO: 67): sequence of Cas9-HR 4 to be used in conjunction with any of the above vectors for bacterial expression; Cas9-HR 8 E. coli codon optimized vector (SEQ ID NO: 68): sequence of Cas9-HR 8 to be used in conjunction with any of the above vectors for bacterial expression; and another Cas9-HR 8 E. coli codon optimized vector (SEQ ID NO: 69): sequence of Cas9-HR 8 to be used in conjunction with any of the above vectors for bacterial expression. SEQ ID NOs: 71-74 illustrate exemplary nucleic acid sequences of constructs for mammalian expression: pX330 (SEQ ID NO: 71): plasmid for dual mammalian U6 driven gRNA and CAG driven Cas9-HR expression; pCAG (SEQ ID NO: 72): plasmid for expression of Cas9-HR via the strong synthetic CAG promoter; pEmpty (SEQ ID NO: 73): vector lacking a promoter. To be replaced by promoters driving tissue specific expression of Cas9-HR; and pCMV (SEQ ID NO: 74): strong constitutive promoter for mammalian expression of Cas9-HR.

SEQ ID NOs: 81-86 (nucleic acid sequences) and SEQ ID NOs: 91-96 (polypeptide sequences) illustrate exemplary nucleic acid or polypeptide sequences of mammalian integration and expression: AAVS1_Cas9-HR8-T2A-NeoR (SEQ ID NO: 81 and SEQ ID NO: 91): for integration of Cas9-HR (8 as example) fused to the coding sequence for neomycin resistance via a self-cleaving peptide (T2A as example) sequence and designed for constitutive genomic expression of Cas9-HR and neomycin resistance at the AAVS1 site; AAVS1_Cas9-HR8-T2A-PuroR (SEQ ID NO: 82 and SEQ ID NO: 92): for integration of Cas9-HR (8 as example) fused to the coding sequence for puromycin resistance via a self-cleaving peptide (T2A as example) sequence and designed for constitutive genomic expression of Cas9-HR and puromycin resistance at the AAVS1 site; AAVS1_Cas9-HR8 (SEQ ID NO: 83 and SEQ ID NO: 93): for integration of Cas9-HR (8 as example) and designed for constitutive genomic expression of Cas9-HR and puromycin resistance at the AAVS1 site; AAVS1_Cas9-HR8-6×His (SEQ ID NO: 84 and SEQ ID NO: 94) for integration of Cas9-HR (8 as example) tagged with a 6×His tag (SEQ ID NO: 236) and designed for constitutive genomic expression of Cas9-HR and puromycin resistance at the AAVS1 site; AAVS1_Cas9-HR8-6×His-C-T2A-PuroR (SEQ ID NO: 85 and SEQ ID NO: 95): for integration of Cas9-HR (8 as example) tagged with a 6×His tag (SEQ ID NO: 236) fused to the coding sequence for puromycin resistance via a self-cleaving peptide (T2A as example) sequence and designed for constitutive genomic expression of Cas9-HR and puromycin resistance at the AAVS1 site; and AAVS1_Cas9-HR8-6×His-C-T2A-NeoR (SEQ ID NO: 86 and SEQ ID NO: 96): for integration of Cas9-HR (8 as example) tagged with a 6×His tag (SEQ ID NO: 236) fused to the coding sequence for neomycin resistance via a self-cleaving peptide (T2A as example) sequence and designed for constitutive genomic expression of Cas9-HR and neomycin resistance at the AAVS1 site.

In some embodiments, the construct comprises any single or combination of components for inducing the HDR. For example, the construct can comprise polynucleotide comprising nucleic acid sequence encoding a promoter, any one of the Cas9-HR or Cas fusion protein described herein, a reporter, at least one guide polynucleotide, a polynucleotide of interest, a selection marker (e.g., antibody resistance selection marker), a fragment thereof, or a combination thereof. In some embodiments, at least one construct is introduced into a cell harboring the endogenous genetic mutation. In some embodiments, at least two different constructs are introducing into a cell harboring the endogenous genetic mutation. In some embodiments, a fragment of the construct can be inserted into a safe harbor site (SHS) of the chromosome. Non-limiting examples of the SHS where the construct can be inserted include SHS_227_chr1_231999396-231999415; SHS_229_chr2_45708354-45708373; SHS_231_chr4_58976613-58976632; SHS_233_chr6_114713905-114713924; SHS_253_chr2_48830185-48830204; SHS_255_chr5_19069307-19069326; SHS_257_chr7_138809594-138809613; SHS_259_chr14_92099558-92099577; SHS_261_chr17_48573577-48573596; SHS_263_chrX_12590812-12590831; SHS_283_chr4_37769238-37769257; SHS_285_chr6_89574320-89574339; SHS_289_chr1_175942362-175942381; SHS_291_chr22_35770121-35770140; SHS_293 chr8_40727927-40727946; SHS_295_chr12_66516386-66516405; SHS_297_chr17_14810285-14810304; SHS_299_chr6_138972461-138972480; SHS_301_chr7_113327685-113327704; SHS_303_chr2_150500675-150500694; SHS_305_chr5_159922029-159922048; SHS_307 chr16_19323777-19323796; SHS_309_chr20_5055245-5055264; SHS_311_chr6_134385946-134385965; SHS_313_chrX_16059732-16059751; SHS_315_chr5_7577728-7577747; SHS_317_chr2_77263930-77263949; SHS_319_chr11_32680546-32680565; SHS_321_chrX_79674328-79674347; SHS_323_chr1_152360840-152360859; SHS_325_chr8_68720172-68720191; SHS_327_chr5_93159222-93159241; SHS_329_chr12_126152581-126152600; SHS_331_chr3_31670871-31670890; SHS_333_chr12_27543737-27543756; SHS_AAVS1_chr19_55625241-55629351; SHS_AAVS1_chr19_55625241-55629351; or SHS_hROSA2_chr3_9415082-9414043.

### Guide Polynucleotide

A ribonucleic acid that comprises a sequence for guiding the ribonucleic acid to a target site on a gene and another sequence for binding to an endonuclease such as Cas9 enzyme is used herein. The guide polynucleotide can comprises at least one CRISPR RNA (crRNA) and at least one transactivating crRNA (tracrRNA). A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid. A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid. The crRNA and tracrRNA can hybridize to form a guide nucleic acid. The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to be used. The Cas protein-binding segment of a guide nucleic acid can comprise two stretches of nucleotides (e.g., crRNA and tracrRNA) that are complementary to one another. The two stretches of nucleotides (e.g., crRNA and tracrRNA) that are complementary to one another can be covalently linked by intervening nucleotides (e.g., a linker in the case of a single guide nucleic acid). The two stretches of nucleotides (e.g., crRNA and tracrRNA) that are complementary to one another can hybridize to form a double stranded RNA duplex or hairpin of the Cas protein-binding segment, thus resulting in a stem-loop structure. The crRNA and the tracrRNA can be covalently linked via the 3′ end of the crRNA and the 5′ end of the tracrRNA. Alternatively, tracrRNA and crRNA can be covalently linked via the 5′ end of the tracrRNA and the 3′ end of the crRNA.

In some cases, the target site is a genomic locus. In some embodiments, the genomic locus encodes a gene. In some embodiments, the genomic locus does not encode a gene. In some embodiments, the genomic locus is a safe harbor site (SHS). SEQ ID NOs 101-117 illustrate exemplary nucleic acid sequences of guide polynucleotide described herein.

In some embodiments, the gRNA is a synthetic gRNA (sgRNA). The gRNA directs the fusion protein complex to a targeted nucleotide sequence of the DNA molecule. The gRNA is a short synthetic RNA composed of a scaffold sequence necessary for Cas-binding and a user-defined about 20 nucleotide spacer that defines the genomic target to be modified. In some embodiments, the gRNA targets a genomic locus that encodes a gene associated with cancer. In some embodiments, the gRNA targets an oncogene. In some embodiments, the gRNA targets an oncogene a tumor suppressor gene. In some embodiments, the gene associated with the cancer is Cadherin. In some embodiments, the gene associated with the cancer is E-Cadherin. In some embodiments, the gene associated with the cancer is Catenin. In some embodiments, the gene associated with the cancer is Beta-Catenin.

In some embodiments, the genomic locus comprises at least one mutation that can be corrected by the fusion protein complex and the polynucleotide of interest described herein. In some embodiments, the genomic locus comprises at least two mutations that can be corrected by the fusion protein complex and the polynucleotide of interest described herein. In some embodiments, the genomic locus comprises at least two mutation, at least three mutations, at least four mutations, at least five mutations, at least ten mutations, at least twenty mutations, at least fifty mutations, at least one hundred mutations, or more mutations that can be corrected by the fusion protein complex inserting the polynucleotide of interest described herein. In some embodiments, the mutations can be spaced apart in the genomic locus by at least 200 base pair (bp), at least 300 bp, at least 400 bp, at least 500 bp, at least 600 bp, at least 700 bp, at least 800 bp, at least 900 bp, at least 1,000 bp, at least 1,100 bp, at least 1,200 bp, at least 1,300 bp, at least 1,400 bp, at least 1,500 bp, at least 1,600 bp, at least 1,700 bp, at least 1,800 bp, at least 1,900 bp, at least 2,000 bp, at least 2,500 bp, at least 3,000 bp, at least 3,500 bp, at least 4,000 bp, at least 4,500 bp, at least 5,000 bp, at least 5,500 bp, at least 6,000 bp, at least 6,500 bp, at least 7,000 bp, at least 7,500 bp, at least 8,000 bp, at least 8,500 bp, at least 9,000 bp, at least 9,500 bp, at least 10,000 bp, at least 11,000 bp, at least 12,000 base pair (bp), at least 13,000 bp, at least 14,000 bp, at least 15,000 bp, at least 16,000 bp, at least 17,000 bp, at least 18,000 bp, at least 19,000 bp, at least 20,000 or more base pairs.

In some embodiments, the guide polynucleotide can direct the Cas9 fusion protein (e.g., any one of the Cas9-HR) to induce insertion of least two, at least three, at least four, at least five, or more polynucleotides of interest into a genomic locus, where the polynucleotide of interest can be the same (e.g., sharing identical nucleic acid sequences) or different (e.g., comprising different nucleic acid sequences for encoding different genes). In some embodiments, the method described herein can utilize multiple guide polynucleotides to direct the Cas9 fusion protein to insert the same polynucleotide of interest in multiple genomic loci. For example, the Cas9 fusion protein can be directed by a plurality of the guide polynucleotides to insert the same polynucleotide of interest two, three four, five, or more genomic loci. In some embodiments, the method described herein can utilize multiple guide polynucleotides to direct the Cas9 fusion protein to insert the same polynucleotide of interest in multiple genomic loci. For example, the Cas9 fusion protein (e.g., any one of the Cas9-HR) can be directed by a plurality of the guide polynucleotides to insert the same polynucleotide of interest to two, three four, five, or more genomic loci. In some embodiments, the method described herein can utilize multiple guide polynucleotides to direct the Cas9 fusion protein to insert the same polynucleotide of interest in multiple genomic loci. For example, the Cas9 fusion protein (e.g., any one of the Cas9-HR) can be directed by a plurality of the guide polynucleotides to insert the same polynucleotide of interest to two, three four, five, or more genomic loci. In some embodiments, the method described herein can utilize multiple guide polynucleotides to direct the Cas9 fusion protein to insert the different polynucleotides of interest in at least one, at least two, or more genomic loci. For example, the Cas9 fusion protein (e.g., any one of the Cas9-HR) can be directed by a plurality of the guide polynucleotides to insert the different polynucleotides of interest to two or more genomic loci.

In some embodiments, the guide polynucleotide can direct the Cas9 fusion protein to induce insertion of the polynucleotide of interest in a genomic locus comprising a safe harbor site (SHS). In some embodiments, at least two, at least three, at least four, at least five, or more polynucleotides of interest can be introduced into a SHS. In some embodiments, the method described herein can utilize multiple guide polynucleotides to direct the Cas9 fusion protein to insert at least one polynucleotide of interest in multiple safe harbor sites. For example, the Cas9 fusion protein can be directed by a plurality of the guide polynucleotides to insert at least one polynucleotide of interest to two, three four, five, or more safe harbor sites. n some embodiments, the method described herein can utilize multiple guide polynucleotides to direct the Cas9 fusion protein to insert the different polynucleotides of interest in at least one, at least two, or more safe harbor sites. For example, the Cas9 fusion protein can be directed by a plurality of the guide polynucleotides to insert the different polynucleotides of interest to two or more safe harbor sites.

### Polynucleotide of Interest

Genome stability necessitates the correct and efficient repair of DSBs. In eukaryotic cells, mechanistic repair of DSBs occurs primarily by two pathways: Non-Homologous End-Joining (NHEJ) and Homology Directed Repair (HDR). NHEJ is the canonical homology-independent pathway as it involves the alignment of only one to a few complementary bases at most for the re-ligation of two ends, whereas HDR uses longer stretches of sequence homology to repair DNA lesions. HDR is the more accurate mechanism for DSB repair due to the requirement of higher sequence homology between the damaged and intact donor strands of DNA. The process is error-free if the DNA template used for repair is identical to the original DNA sequence at the DSB, or it can introduce very specific mutations into the damaged DNA.

As addressed above, HDR methods provide the great freedom in genomic engineering, allowing for as little as single base mutations and up to insertions or deletions of kilo-bases (kb) of DNA. In eukaryotes, HDR rate is governed by the competition between two different pathways: Homologous Recombination (HR) and Non-Homologous End Joining (NHEJ). The competition between these two pathways begins by competitive binding by either MRN/CtIP complex or Ku 70/80 heterodimer. If MRN/CtIP bind first, they recruit other proteins, including Exonuclease I (ExoI), which possess 5′->3′ exonuclease activity 20. 5′ end resection of double strand DNA breaks by either Exo1 or Dna2 at each side of the break commits the DSB to be repaired by the HR pathway. Alternatively, if the Ku 70/80 heterodimer binds, it can then recruit other NHEJ pathway members, including DNA Ligase IV, and eventually repairs the double strand break via NHEJ.

Polynucleotide of interest comprising a HDR template sequences is needed to be delivered into a cell when delivering the CRISPR-Cas9 system to the cell. HDR templates used to create specific mutations or insert new elements into a gene require a certain amount of homology surrounding the target sequence that will be modified. In some embodiments, the 5′ and 3′ homology arms start at the CRISPR-induced DSB. In general, the insertion sites of the modification can be very close to the DSB, ideally less than 10 bp away if possible. In some embodiments, the 5′ and 3′ homology arm of the HDR template sequences are at least 80% identical to the targeted sequence. Further, in some embodiments, single stranded donor oligonucleotide (ssDON) is utilized for smaller insertions. Each homology arm of the ssDON may comprise about 30-80 bp nucleotide sequence. The length of the homology arm is not meant to be limiting and the length can be adjusted by a person of ordinary skill in the art according to a locus of gene interest and experimental system. For larger insertions such as fluorescent proteins or selection cassettes, double stranded donor oligonucleotide (dsDON) can be utilized as HDR template sequence. In some embodiments, each homology arm of the ssDON may comprise about 800-1500 bp nucleotide sequence. To prevent Cas9 enzyme cleaving the HDR template, in some embodiments, a single base mutation can be introduced in the Protospacer Adjacent Motif (PAM) sequence of the HDR template.

In some embodiments, the polynucleotide of interest to be inserted comprises a length that is at least 1000 nucleotides, at least 1500 nucleotides, at least 2000 nucleotides, at least 2500 nucleotides, at least 3000 nucleotides, at least 3500 nucleotides, at least 4000 nucleotides, at least 4500 nucleotides, at least 5000 nucleotides, at least 5500 nucleotides, or more nucleotides. In some embodiments, the polynucleotide of interest to be inserted comprises a length that i about 1,000 nucleotides to about 6,500 nucleotides. In some embodiments, the polynucleotide of interest to be inserted comprises a length that is about 1,000 nucleotides to about 1,500 nucleotides, about 1,000 nucleotides to about 2,000 nucleotides, about 1,000 nucleotides to about 2,500 nucleotides, about 1,000 nucleotides to about 3,000 nucleotides, about 1,000 nucleotides to about 3,500 nucleotides, about 1,000 nucleotides to about 4,000 nucleotides, about 1,000 nucleotides to about 4,500 nucleotides, about 1,000 nucleotides to about 5,000 nucleotides, about 1,000 nucleotides to about 5,500 nucleotides, about 1,000 nucleotides to about 6,000 nucleotides, about 1,000 nucleotides to about 6,500 nucleotides, about 1,500 nucleotides to about 2,000 nucleotides, about 1,500 nucleotides to about 2,500 nucleotides, about 1,500 nucleotides to about 3,000 nucleotides, about 1,500 nucleotides to about 3,500 nucleotides, about 1,500 nucleotides to about 4,000 nucleotides, about 1,500 nucleotides to about 4,500 nucleotides, about 1,500 nucleotides to about 5,000 nucleotides, about 1,500 nucleotides to about 5,500 nucleotides, about 1,500 nucleotides to about 6,000 nucleotides, about 1,500 nucleotides to about 6,500 nucleotides, about 2,000 nucleotides to about 2,500 nucleotides, about 2,000 nucleotides to about 3,000 nucleotides, about 2,000 nucleotides to about 3,500 nucleotides, about 2,000 nucleotides to about 4,000 nucleotides, about 2,000 nucleotides to about 4,500 nucleotides, about 2,000 nucleotides to about 5,000 nucleotides, about 2,000 nucleotides to about 5,500 nucleotides, about 2,000 nucleotides to about 6,000 nucleotides, about 2,000 nucleotides to about 6,500 nucleotides, about 2,500 nucleotides to about 3,000 nucleotides, about 2,500 nucleotides to about 3,500 nucleotides, about 2,500 nucleotides to about 4,000 nucleotides, about 2,500 nucleotides to about 4,500 nucleotides, about 2,500 nucleotides to about 5,000 nucleotides, about 2,500 nucleotides to about 5,500 nucleotides, about 2,500 nucleotides to about 6,000 nucleotides, about 2,500 nucleotides to about 6,500 nucleotides, about 3,000 nucleotides to about 3,500 nucleotides, about 3,000 nucleotides to about 4,000 nucleotides, about 3,000 nucleotides to about 4,500 nucleotides, about 3,000 nucleotides to about 5,000 nucleotides, about 3,000 nucleotides to about 5,500 nucleotides, about 3,000 nucleotides to about 6,000 nucleotides, about 3,000 nucleotides to about 6,500 nucleotides, about 3,500 nucleotides to about 4,000 nucleotides, about 3,500 nucleotides to about 4,500 nucleotides, about 3,500 nucleotides to about 5,000 nucleotides, about 3,500 nucleotides to about 5,500 nucleotides, about 3,500 nucleotides to about 6,000 nucleotides, about 3,500 nucleotides to about 6,500 nucleotides, about 4,000 nucleotides to about 4,500 nucleotides, about 4,000 nucleotides to about 5,000 nucleotides, about 4,000 nucleotides to about 5,500 nucleotides, about 4,000 nucleotides to about 6,000 nucleotides, about 4,000 nucleotides to about 6,500 nucleotides, about 4,500 nucleotides to about 5,000 nucleotides, about 4,500 nucleotides to about 5,500 nucleotides, about 4,500 nucleotides to about 6,000 nucleotides, about 4,500 nucleotides to about 6,500 nucleotides, about 5,000 nucleotides to about 5,500 nucleotides, about 5,000 nucleotides to about 6,000 nucleotides, about 5,000 nucleotides to about 6,500 nucleotides, about 5,500 nucleotides to about 6,000 nucleotides, about 5,500 nucleotides to about 6,500 nucleotides, or about 6,000 nucleotides to about 6,500 nucleotides. In some embodiments, the polynucleotide of interest to be inserted comprises a length that is about 1,000 nucleotides, about 1,500 nucleotides, about 2,000 nucleotides, about 2,500 nucleotides, about 3,000 nucleotides, about 3,500 nucleotides, about 4,000 nucleotides, about 4,500 nucleotides, about 5,000 nucleotides, about 5,500 nucleotides, about 6,000 nucleotides, or about 6,500 nucleotides. In some embodiments, the polynucleotide of interest to be inserted comprises a length that is at least about 1,000 nucleotides, about 1,500 nucleotides, about 2,000 nucleotides, about 2,500 nucleotides, about 3,000 nucleotides, about 3,500 nucleotides, about 4,000 nucleotides, about 4,500 nucleotides, about 5,000 nucleotides, about 5,500 nucleotides, or about 6,000 nucleotides. In some embodiments, the polynucleotide of interest to be inserted comprises a length that is at most about 1,500 nucleotides, about 2,000 nucleotides, about 2,500 nucleotides, about 3,000 nucleotides, about 3,500 nucleotides, about 4,000 nucleotides, about 4,500 nucleotides, about 5,000 nucleotides, about 5,500 nucleotides, about 6,000 nucleotides, or about 6,500 nucleotides.

In some embodiments, the polynucleotide of interest comprises a repair template (e.g., a HDR template described herein). In some embodiments, the repair template encodes a wide type gene or a fragment thereof for correcting at least one mutation in the genomic locus. In some embodiments, the repair template comprises a length that is sufficient to correct at least two mutations, where the mutations are spaced at least 200 bp, at least 300 bp, at least 400 bp, at least 500 bp, at least 600 bp, at least 700 bp, at least 800 bp, at least 900 bp, at least 1000 bp, at least 1100 bp, at least 1200 bp, at least 1300 bp, at least 1400 bp, at least 1500 bp, at least 1600 bp, at least 1700 bp, at least 1800 bp, at least 1900 bp, at least 2000 bp, at least 2500 bp, at least 3000 bp, at least 3500 bp, or more base pairs.

In some embodiments, the polynucleotide of interest can encode a full length transgene. In some embodiments, the polynucleotide of interest can encode a fragment of a transgene. In some embodiments, the polynucleotide of interest can encode a reporter. For example, the polynucleotide of interest can encode a reporter for diagnosing a disease or condition described herein. In some embodiments, the polynucleotide of interest can encode a regulatory element for regulating gene expression in a cell. For example, the polynucleotide of interest can encode at least one RNA such as a transfer RNA (tRNA), a ribosomal RNA (rRNA), an snRNA, a long non-coding RNA, a small RNA, a snoRNA, a siRNA, a miRNA, a tRNA-derived small RNA (tsRNA), an antisense RNA, an shRNA, or a small rDNA-derived RNA (srRNA) for modulating gene expression of an endogenous gene in a cell.

### Method

### Delivery

The construct described herein can be introduced into a cell by any method for delivering the composition described herein into the cell. In some embodiments, the construct is introduced into the cell by physical, chemical, or biological methods.

Physical methods for introducing a construct into a host cell include calcium phosphate precipitation, lipofection, particle bombardment, microinjection, gene gun, electroporation, and the like. Methods for producing cells comprising vectors and/or exogenous nucleic acids are suitable for methods herein. One method for the introduction of a construct into a host cell is calcium phosphate transfection.

Biological methods for introducing a construct into a host cell include the use of DNA and RNA vectors. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector.

Chemical means for introducing a construct into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the construct into a host cell (in vitro, ex vivo, or in vivo). In another aspect, the nucleic acid is associated with a lipid. The construct associated with a lipid, in some embodiments, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape.

### Modification of Genomic Locus

Described herein, in some embodiments, are methods for modifying a genomic locus by inserting a polynucleotide of interest described herein. In some embodiments, the method comprises contacting a cell with a Cas fusion protein complex comprising a Cas fusion protein (e.g., any one of the Cas9-HR) complexed with a guide polynucleotide configured to bind to a genomic locus of the cell. In some embodiments, the method further comprises contacting the same cell with a polynucleotide of interest (e.g., a repair template) comprising a nucleic acid donor sequence that is at least 1000 nucleotides, at least 1500 nucleotides, at least 2000 nucleotides, at least 2500 nucleotides, at least 3000 nucleotides, at least 3500 nucleotides, at least 4000 nucleotides, at least 4500 nucleotides, at least 5000 nucleotides, at least 5500 nucleotides, or more nucleotides in length. In some embodiments, the Cas fusion protein complex is configured to induce homology-directed repair (HDR) of the genomic locus of a cell with the nucleic acid donor sequence to produce an edited cell. In some embodiments, the editing of the cell can be used to treat a disease or condition. In some embodiments, the edited cell can be further formulated into a pharmaceutical composition for treating the disease or condition.

In some embodiments, the method can correct at least one mutation in a genomic locus, where the genomic locus encodes a gene associated with the cancer. For example, the genomic locus comprising the at least one mutation can encode Cadherin or Catenin. In some embodiments, the gene associated with the cancer is an oncogene. In some embodiments, the gene associated with the cancer is a tumor suppressor gene.

In some embodiments, the method inserts at least one polynucleotide of interest into a safe harbor site (SHS). In some embodiments, the method inserts a least one polynucleotide of interest comprising a repair template or HDR template into the SHS. In some embodiments, the polynucleotide of interest encodes a full length transgene or a fragment of the transgene. For example, the method described herein can insert a polynucleotide of interest encoding full length Cadherin or Catenin. In some embodiment, the transgene gene can be a reporter for diagnosing a disease or condition described herein.

In some embodiments, the method described herein increases a HDR editing rate in a plurality of the cells contacted with Cas9 fusion protein compared to a HDR editing rate induced by a conventional or wild type Cas9 in a comparable plurality of cells. In some embodiments, the HDR editing rate induced by the Cas9 fusion protein is increased by least 10%, at least 50%, at least 100%, at least 200%, at least 300%, or more compared the HDR editing rate induced by the conventional or the wild type Cas9.

In some embodiments, the method described herein increases a HDR editing rate in a plurality of the cells contacted with Cas9 fusion protein (e.g., any one of the Cas9-HR) compared to a HDR editing rate induced by a conventional or wild type Cas9 in a comparable plurality of cells. In some embodiments, the HDR editing rate induced by the Cas9 fusion protein is increased by least 10%, at least 50%, at least 100%, at least 200%, at least 300%, or more compared the HDR editing rate induced by the conventional or the wild type Cas9.

In some embodiments, the method described herein increases cell viability in a plurality of the cells contacted with Cas9 fusion protein (e.g., any one of the Cas9-HR) compared to cell viability of a comparable plurality of cells contacted a conventional or wild type Cas9 in. In some embodiments, the cell viability of the plurality of cells contacted with the Cas9 fusion protein is increased by least 10%, at least 50%, at least 100%, at least 200%, at least 300%, or more compared the cell viability of the comparable plurality of cells contacted the conventional or the wild type Cas9.

In some embodiments, the method described herein decreases cellular toxicity in a plurality of the cells contacted with Cas9 fusion protein (e.g., any one of the Cas9-HR) compared to cellular toxicity of a comparable plurality of cells contacted a conventional or wild type Cas9 in. In some embodiments, the cellular toxicity of the plurality of cells contacted with the Cas9 fusion protein is increased by least 10%, at least 50%, at least 100%, at least 200%, at least 300%, or more compared the cellular toxicity of the comparable plurality of cells contacted the conventional or the wild type Cas9.

In some embodiments, the method described herein decreases endogenous p53 signaling in a cell contacted with Cas9 fusion protein (e.g., any one of the Cas9-HR) compared to endogenous p53 signaling of a comparable cell contacted with a conventional or wild type Cas9. In some embodiments, the endogenous p53 signaling in the cell contracted with the Cas9 fusion protein is decreased by at least 10%, at least 50%, at least 100%, at least 200%, at least 300%, or more compared to the endogenous p53 of the comparable cell contacted with the conventional or wild type Cas9. In some embodiments, the decreased p53 signaling as induced by the Cas9 fusion protein (e.g., any one of the Cas9-HR) leads to an increase of cellular viability. In some embodiments, the decreased p53 signaling as induced by the Cas9 fusion protein (e.g., any one of the Cas9-HR) leads to a decrease of cellular toxicity. In some embodiments, the decreased p53 signaling as induced by the Cas9 fusion protein (e.g., any one of the Cas9-HR) leads to an increase of the HDR rate. In some embodiments, the decreased p53 signaling as induced by the Cas9 fusion protein (e.g., any one of the Cas9-HR), when combined with the modification of the genomic locus induced by the method described herein, can decrease cellular proliferation. In some embodiments, the decreased p53 signaling as induced by the Cas9 fusion protein (e.g., any one of the Cas9-HR), when combined with the modification of the genomic locus induced by the method described herein, can decrease cellular migration such as metastasis.

### Treatment

Disclosed herein, in some embodiments, are methods for treating a disease or condition by inserting at least one polynucleotide of interest into a genomic locus via the use of the Cas fusion protein (e.g., any one of Cas9-HR described herein). In some embodiment, the method comprises contacting a cell with the Cas fusion protein, Cas fusion protein complex, guide polynucleotide, polynucleotide of interest, or a combination thereof to correct at least one mutation encoded by the genomic locus. In some embodiments, the genomic locus encodes a gene associated with the disease or condition. In some embodiments, the method comprises administering the Cas fusion protein, Cas fusion protein complex, guide polynucleotide, polynucleotide of interest, or a combination thereof to a subject in need thereof. In some embodiments, the method comprises editing a cell with the Cas fusion protein, Cas fusion protein complex, guide polynucleotide, polynucleotide of interest, or a combination thereof to generate an edited cell and then subsequently administering the edited cell to the subject. In some embodiments, the Cas fusion protein, Cas fusion protein complex, guide polynucleotide, polynucleotide of interest, or a combination thereof, or the edited cell can formulated into a pharmaceutical composition to be administered to the subject.

In some embodiments, the Cas fusion protein, Cas fusion protein complex, guide polynucleotide, polynucleotide of interest, or a combination thereof, or the edited cell, or the pharmaceutical composition can be administered to the subject alone (e.g., standalone treatment). In some embodiments, the Cas fusion protein, Cas fusion protein complex, guide polynucleotide, polynucleotide of interest, or a combination thereof, or the edited cell, or the pharmaceutical composition is administered in combination with an additional agent. In some cases, the additional agent as used herein is administered alone. the Cas fusion protein, Cas fusion protein complex, guide polynucleotide, polynucleotide of interest, or a combination thereof, or the edited cell, or the pharmaceutical composition and the additional agent can be administered together or sequentially as a combination therapy. The combination therapy can be administered within the same day, or can be administered one or more days, weeks, months, or years apart. In some embodiments, the additional agent is a p53 inhibitor.

In some embodiments, the Cas fusion protein, Cas fusion protein complex, guide polynucleotide, polynucleotide of interest, or a combination thereof, or the edited cell, or the pharmaceutical composition is a first-line treatment for the disease or condition. In some embodiments, the Cas fusion protein, Cas fusion protein complex, guide polynucleotide, polynucleotide of interest, or a combination thereof, or the edited cell, or the pharmaceutical composition is a second-line, third-line, or fourth-line treatment. In some embodiments, the Cas fusion protein, Cas fusion protein complex, guide polynucleotide, polynucleotide of interest, or a combination thereof, or the edited cell, or the pharmaceutical composition comprises at least one, two, three, four, five, six, seven, eight, nine, 10, 20, 30 or more guide polynucleotides or polynucleotides of interest. In some instances, method comprises administering the Cas fusion protein, Cas fusion protein complex, guide polynucleotide, polynucleotide of interest, or a combination thereof, or the edited cell, or the pharmaceutical composition by intravenous (“i.v.”) administration. It is conceivable that one can also administer the Cas fusion protein, Cas fusion protein complex, guide polynucleotide, polynucleotide of interest, or a combination thereof, or the edited cell, or the pharmaceutical composition disclosed herein by other routes, such as subcutaneous injection, intramuscular injection, intradermal injection, transdermal injection percutaneous administration, intranasal administration, intralymphatic injection, rectal administration intragastric administration, or any other suitable parenteral administration. In some embodiments, routes for local delivery closer to site of injury or inflammation are preferred over systemic routes. Routes, dosage, time points, and duration of administrating therapeutics can be adjusted. In some embodiments, administration of therapeutics is prior to, or after, onset of either, or both, acute and chronic symptoms of the disease or condition.

Suitable dose and dosage administrated to a subject is determined by factors including, but no limited to, the particular the Cas fusion protein, Cas fusion protein complex, guide polynucleotide, polynucleotide of interest, or a combination thereof, or the edited cell, or the pharmaceutical composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject being treated.

In some embodiments, the administration of the Cas fusion protein, Cas fusion protein complex, guide polynucleotide, polynucleotide of interest, or a combination thereof, or the edited cell, or the pharmaceutical composition described herein is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges can be adjusted based on subject's response to the treatment. Some routes of administration will require higher concentrations of effective amount of therapeutics than other routes.

In some embodiments, the administration of the Cas fusion protein, Cas fusion protein complex, guide polynucleotide, polynucleotide of interest, or a combination thereof, or the edited cell, or the pharmaceutical composition described herein increases survival rate of the subject by at least 1%, 2%, 3%, 4%, 5%, 6%, 7%, 8%, 9%, 10%, 15%, 20%, 30%, 40%, 50%, or more. In some embodiments, the administration of the Cas fusion protein, Cas fusion protein complex, guide polynucleotide, polynucleotide of interest, or a combination thereof, or the edited cell, or the pharmaceutical composition described herein at a dose that increases survival rate of the subject by at least 1%, 2%, 3%, 4%, 5%, 6%, 7%, 8%, 9%, 10%, 15%, 20%, 30%, 40%, 50%, or more. In some embodiments, the administration of the Cas fusion protein, Cas fusion protein complex, guide polynucleotide, polynucleotide of interest, or a combination thereof, or the edited cell, or the pharmaceutical composition described herein at a schedule that increases survival rate of the subject by at least 1%, 2%, 3%, 4%, 5%, 6%, 7%, 8%, 9%, 10%, 15%, 20%, 30%, 40%, 50%, or more. In some embodiments, the administration of the the Cas fusion protein, Cas fusion protein complex, guide polynucleotide, polynucleotide of interest, or a combination thereof, or the edited cell, or the pharmaceutical composition described herein at a dose and a schedule that increase survival rate of the subject by at least 1%, 2%, 3%, 4%, 5%, 6%, 7%, 8%, 9%, 10%, 15%, 20%, 30%, 40%, 50%, or more.

In some embodiments, the administration of the Cas fusion protein, Cas fusion protein complex, guide polynucleotide, polynucleotide of interest, or a combination thereof, or the edited cell, or the pharmaceutical composition described herein inhibits growth of the tumor by at least 1%, 2%, 3%, 4%, 5%, 6%, 7%, 8%, 9% 10%, 15%, 20%, 30%, 40%, 50%, or more. In some embodiments, the administration of the Cas fusion protein, Cas fusion protein complex, guide polynucleotide, polynucleotide of interest, or a combination thereof, or the edited cell, or the pharmaceutical composition described herein at a dose that inhibits growth of the tumor by at least 1%, 2%, 3% 4%, 5%, 6%, 7%, 8%, 9%, 10%, 15%, 20%, 30%, 40%, 50%, or more. In some embodiments, the administration of the Cas fusion protein, Cas fusion protein complex, guide polynucleotide, polynucleotide of interest, or a combination thereof, or the edited cell, or the pharmaceutical composition described herein at a schedule that inhibits growth of the tumor by at least 1%, 2%, 3%, 4%, 5%, 6%, 7%, 8%, 9%10%, 15%, 20%, 30%, 40%, 50%, or more. In some embodiments, the administration of the Cas fusion protein, Cas fusion protein complex, guide polynucleotide, polynucleotide of interest, or a combination thereof, or the edited cell, or the pharmaceutical composition described herein at a dose and a schedule that inhibits growth of the tumor by at least 1%, 2%, 3%, 4%, 5%, 6%, 7%, 8%, 9%, 10%, 15%, 20%, 30%, 40%, 50%, or more.

In some embodiments, the administration of the Cas fusion protein, Cas fusion protein complex, guide polynucleotide, polynucleotide of interest, or a combination thereof, or the edited cell, or the pharmaceutical composition described herein to the subject in a dose that is sufficient to inhibit growth of the tumor. In some embodiments, the administration of the Cas fusion protein, Cas fusion protein complex, guide polynucleotide, polynucleotide of interest, or a combination thereof, or the edited cell, or the pharmaceutical composition described herein to the subject in a schedule that is sufficient to inhibit growth of the tumor. In some embodiments, the administration of the Cas fusion protein, Cas fusion protein complex, guide polynucleotide, polynucleotide of interest, or a combination thereof, or the edited cell, or the pharmaceutical composition described herein to the subject in a dose and a schedule that are sufficient to inhibit growth of the tumor.

In certain embodiments, where the subject's condition does not improve, upon the doctor's discretion the administration of the Cas fusion protein, Cas fusion protein complex, guide polynucleotide, polynucleotide of interest, or a combination thereof, or the edited cell, or the pharmaceutical composition is administered chronically, that is, for an extended period of time, including throughout the duration of the subject's life in order to ameliorate or otherwise control or limit the symptoms of the subject's disease or condition. In certain embodiments where a subject's status does improve, the dose of the Cas fusion protein, Cas fusion protein complex, guide polynucleotide, polynucleotide of interest, or a combination thereof, or the edited cell, or the pharmaceutical composition being administered can be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of the pharmaceutical composition being administered can be temporarily reduced or temporarily suspended for a certain length of time (i.e., a “drug diversion”).

In some embodiments, once improvement of the subject's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the subject requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the composition described herein lies within a range of circulating concentrations that include the ED50 with minimal toxicity. In certain embodiments, the daily dosage range and/or the unit dosage amount varies within this range depending upon the dosage form employed and the route of administration utilized.

In some embodiments, the disease or condition described herein is a cancer. In some embodiments, the cancer is associated with SOS1. In some embodiments, the cancer is associated with SOS2. In some embodiments, the cancer is associated with KRAS. In some embodiments, the cancer is associated with an abnormality of KRAS-mediated signaling pathway. In some embodiments, the cancer is a lung cancer, a pancreatic cancer, or a colon cancer. Other non-limiting examples of the cancer can include Acute Lymphoblastic Leukemia, Acute Lymphocytic Leukemia (ALL), Acute Myeloid Leukemia (AML), Adenoid Cystic Carcinoma, Adrenal Gland Cancer, Adrenocortical Carcinoma, Adult Leukemia, AIDS-Related Lymphoma, Amyloidosis, Anal Cancer, Astrocytomas, Ataxia Telangiectasia, Atypical Mole Syndrome, Atypical Teratoid/Rhabdoid Tumor, Basal Cell Carcinoma, Bile Duct Cancer, Birt Hogg Dube Syndrome, Bladder Cancer, Bone Cancer, Brain Tumor, Breast Cancer, Bronchial Tumors, Burkitt Lymphoma, Carcinoid Tumor (Gastrointestinal), Carcinoma of Unknown Primary, Cardiac (Heart) Tumors, Cervical Cancer, Cholangiocarcinoma, Chordoma, Chronic Lymphocytic Leukemia (CLL), Chronic Myelogenous Leukemia, Chronic Myeloid Leukemia, Chronic Myeloproliferative Neoplasms, Colorectal Cancer, Craniopharyngioma, Cutaneous T-Cell Lymphoma, Ductal Carcinoma, Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Fallopian Tube Cancer, Fibrous Histiocytoma of Bone, Malignant, and Osteosarcoma, Gallbladder Cancer, Gastric Cancer, Gastrointestinal Carcinoid Tumor, Gastrontestinal Stromal Tumor (GIST), Germ Cell Tumors, Gestational Trophoblastic Disease, Hairy Cell Leukemia, Head and Neck Cancer, Hepatocellular Cancer, HER2-Positive Breast Cancer, Histiocytosis, Langerhans Cell, Hodgkin's Lymphoma, Hypopharyngeal Cancer, Intraocular Melanoma, Islet Cell Tumor, Juvenile Polyposis Syndrome, Kaposi Sarcoma, Kidney Cancer, Langerhans Cell Histiocytosis, Laryngeal Cancer, Leukemia, Lip and Oral Cavity Cancer, Liver Cancer, Lobular Carcinoma, Lung Cancer (Non-Small Cell and Small Cell), Lymphoma, Malignant Fibrous Histiocytoma of Bone and Osteosarcoma, Malignant Glioma, Melanoma, Intraocular Melanoma, Meningioma, Merkel Cell Carcinoma, Mesothelioma, Malignant, Metastatic Cancer, Metastatic Squamous Neck Cancer with Occult Primary, Midline Tract Carcinoma, Multiple Endocrine Neoplasia Syndromes, Multiple Myeloma, Plasma Cell Neoplasms, Mycosis Fungoides, Myelodysplastic Syndrome (MDS), Myeloproliferative Neoplasms, Chronic, Nasal Cavity and Paranasal Sinus Cancer, Nasopharyngeal Cancer, Neuroblastoma, Neuroendocrine Tumor, Non-Hodgkin Lymphoma, Oral Cancer, Lip and Oral Cavity Cancer and Oropharyngeal Cancer, Oropharyngeal Cancer, Osteosarcoma, Ovarian Cancer, Ovarian Germ Cell Tumors, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Peritoneal Cancer, Peutz-Jeghers Syndrome, Pharyngeal Cancer, Pheochromocytoma, Pituitary Tumor, Plasma Cell Neoplasm/Multiple Myeloma, Pleuropulmonary Blastoma, Polycythemia Vera, Pregnancy and Breast Cancer, Primary Central Nervous System (CNS) Lymphoma, Primary Peritoneal Cancer, Prostate Cancer, Rectal Cancer, Recurrent Cancer, Renal Cell Carcinoma, Retinoblastoma, Rhabdomyosarcoma, Salivary Gland Cancer, Sarcoma, Sezary Syndrome, Skin Cancer, Small Intestine Cancer, Soft Tissue Sarcoma, Solid tumor, Squamous Cell Carcinoma of the Skin, Squamous Neck Cancer with Occult Primary, Metastatic, Stomach Cancer, T-Cell Lymphoma, Testicular Cancer, Throat Cancer, Thymoma, Thymic Carcinoma, Thyroid Cancer, Transitional Cell Cancer of the Renal Pelvis and Ureter, Unusual Cancers of Childhood, Ureter and Renal Pelvis, Transitional Cell Cancer, Urethral Cancer, Uterine (Endometrial) Cancer, Uterine Sarcoma, Vaginal Cancer, Vascular Tumors, Vulvar Cancer, Wilms Tumor, or a combination thereof.

### Pharmaceutical Composition

Described herein, in some embodiments, are pharmaceutical compositions comprising the fusion protein, the guide polynucleotide, the polynucleotide of interest (e.g., a repair template,) or a combination thereof. In some embodiments, the pharmaceutical composition comprises a cell edited with the fusion protein described herein. Exemplary cells (with ATCC cell line number) that can be edited and formulated into the pharmaceutical composition can include Embryonic Stem Cells: SRC-2002; Dermal Fibroblasts: PCS-201-010; Mixed Renal Epithelial: PCS-400-012; Corneal Cells: PCS-700-010; Bladder smooth muscle cells: PCS-420-012; Lobar Epithelial Cells: PCS-300-015; Primary Epithelial Cells: PCS-600-010; Adipose derived Mesenchymal Stem Cells: PSC-500-011; Primary Subcutaneous Pre-adipocytes: PCS-210-010; Aortic Endothelial Cells: PCS-100-011; Epidermal Keratinocytes: PCS-200-010; Gingival Keratinocytes: PCS-200-014; Epidermal Melanocytes: PCS-200-012; Coronary Artery Smooth Muscle Cells: PCS-100-021; Lung Smooth Muscle Cells: PCS-130-010; and CD34+: PCS-800-012.

In some embodiments, the pharmaceutical composition further comprises a pharmaceutically acceptable: carrier, excipient, diluent, or nebulized inhalant. In some embodiments, the pharmaceutical composition includes two or more active agents (e.g., a Cas fusion protein complex and the polynucleotide of interest described herein). In some embodiments, the two or more active agents are contained in a single dosage unit. In embodiments, the two or more active agents are contained in separate dosage units. In practicing the methods of treatment or use provided herein, therapeutically effective amounts of pharmaceutical composition described herein is administered to a mammal having a disease, disorder, or condition to be treated, e.g., cancer. In some embodiments, the mammal is a human. A therapeutically effective amount may vary widely depending on the severity of the disease, the age and relative health of the subject, the potency of the therapeutic agent used and other factors. The therapeutic agents, and in some cases, compositions described herein, may be used singly or in combination with one or more therapeutic agents as components of mixtures.

The compositions (e.g., pharmaceutical compositions) described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical composition may be manufactured in a conventional manner, such as, by way of example only, by means of conventional mixing, dissolving, granulating, dragee-making, levigating, emulsifying, encapsulating, entrapping or compression processes.

The pharmaceutical composition may include at least an exogenous therapeutic agent as an active ingredient in free-acid or free-base form, or in a pharmaceutically acceptable salt form. In addition, the methods and compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In certain embodiments, pharmaceutical composition provided herein includes one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, pharmaceutical composition described herein benefits from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, I about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

The pharmaceutical composition described herein is formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

For intravenous injections or drips or infusions, a pharmaceutical composition described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. pharmaceutical composition for injection may be presented in unit dosage form, e.g., in ampoules or in multi dose containers, with an added preservative. The composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a pharmaceutical composition is formulated for use as an aerosol, a mist or a powder. Pharmaceutical compositions described herein are conveniently delivered in the form of an aerosol spray presentation from pressurized packs or nebulizers, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch. Formulations that include a composition are prepared as solutions in saline, employing benzyl alcohol or other suitable preservatives, fluorocarbons, and/or other solubilizing or dispersing agents known in the art. Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compositions described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

In some embodiments, the pharmaceutical composition are provided that include particles of a therapeutic agent and at least one dispersing agent or suspending agent for oral administration to a subject. The formulations may be a powder and/or granules for suspension, and upon admixture with water, a substantially uniform suspension is obtained.

Furthermore, the pharmaceutical composition optionally includes one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the pharmaceutical composition optionally includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other the pharmaceutical composition optionally includes one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, the pharmaceutical composition described herein includes at least one additional active agent other than the enucleated cell described herein. In some embodiments, the at least one additional active agent is a chemotherapeutic agent, cytotoxic agent, cytokine, growth-inhibitory agent, anti-hormonal agent, anti-angiogenic agent, cardio protectant, and/or checkpoint inhibitor.

### Kit

Disclosed herein, in some embodiments, are kits for using the compositions described herein. In some embodiments, the kits disclosed herein may be used to treat a disease or condition in a subject. In some embodiments, the kits comprise an assemblage of materials or components apart from the composition.

In some embodiments, the kits described herein comprise components for synthesizing the constructs or vectors described herein for encoding the fusion protein, guide polynucleotide, polynucleotide of interest (e.g., a repair template), or a combination thereof. In some embodiments, the kits described herein comprise components for delivering the constructs or vectors described herein into a cell. In some embodiments, the kits described herein comprise components for selecting for a homogenous population of the edited cells. In some embodiments, the kits described herein comprise components for selecting for a heterogenous population of the edited cells. In some embodiments, the kit comprises components for performing assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., cancer) in a subject. In some embodiments, the kit is configured particularly for the purpose of treating mammalian subjects. In some embodiments, the kit is configured particularly for the purpose of treating human subjects.

Instructions for use may be included in the kit. In some embodiments, the kit comprises instructions for administering the composition to a subject in need thereof. In some embodiments, the kit comprises instructions for further engineering the composition to express a biomolecule (e.g., the fusion protein described herein). In some embodiments, the kit comprises instructions thawing or otherwise restoring biological activity of the composition, which may have been cryopreserved, lyophilized, or cryo-hibernated during storage or transportation. In some embodiments, the kit comprises instructions for measure viability of the restored compositions, to ensure efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components are typically contained in suitable packaging material(s).

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular and not limited to sequential steps. Accordingly, terms such as “first” and “second” do not necessarily imply priority, order of importance, or order of acts.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Example 1. Design and Characterization of Cas9-HRs

A fragment of Human Exo1 (1-352), which possess exonuclease activity but lacks post-translational regulation, was N-terminally fused to Cas9 with linkers of different lengths and amino acids, which are diagrammed in FIG. 1A. Two different versions of Cas9 were used: one with two nucleus localizing sequences (2XNLS)-Cas9, and one with the N-terminal NLS deleted, 1XNLS-Cas9, hypothesizing that the extra NLS could interfere with proper fusion of hExo1 (FIG. 1A). Finally, a directly fused hExo1-Cas9 construct was developed as well, as it is possible for linkers to have a deleterious effect on fusion protein performance. Here after the hExo1(1-352)-Cas9 fusions will be referred to Cas9-HR 1-9. Plasmid PX330 was chosen as the expression vector, as it allows for fascicle simultaneous expression of Cas9 and gRNA, diagramed in FIG. 1B, top. After constructs were cloned and sequenced, Cas9-HRs 1-8 were then tested in Human lung carcinoma A549 cells. A549 cells were chosen as they are both facile to grow and transfect, cost effective in terms of media and other reagents, and importantly have retained a functional p53 gene. An intergenic region on Chromosome 12 was targeted, with the idea that if Cas9-HRs can shift cells from NHEJ to HR repair, p53 pathway activation and corresponding cell death should be reduced compared to unmodified Cas9.

Cells were plated in 96 well plates and transfected with Cas9-HR 1-8, Cas9 and GFP using a standard Calcium Phosphate transfection technique and incubated overnight for 16-20 hours (FIG. 1B, bottom). Cells were then allowed to recover for one additional day, after which viability was cells were incubated with resazurin (Millipore Sigma) for 4 hours, after which fluorescence was quantified by a plate reader (Perkin Elmer). FIG. 1C shows that all Cas9-HR fusions had greatly increased and statistically significant increased cellular viability (˜2-4 fold) compared to unmodified Cas9. Dramatically, most had similar survival rates when compared to GFP, indicating that Cas9-HRs may significantly reduce NHEJ repair and subsequent p53 pathway activation. Importantly, hExo1 fusion to Cas9 is required, as co-expression of Cas9 and hExo1 was insufficient to reduce Cas9 mediated cellular toxicity.

### Example 2. Cas9 Cellular Toxicity in A549 is in Part Mediated by the p53 Pathway

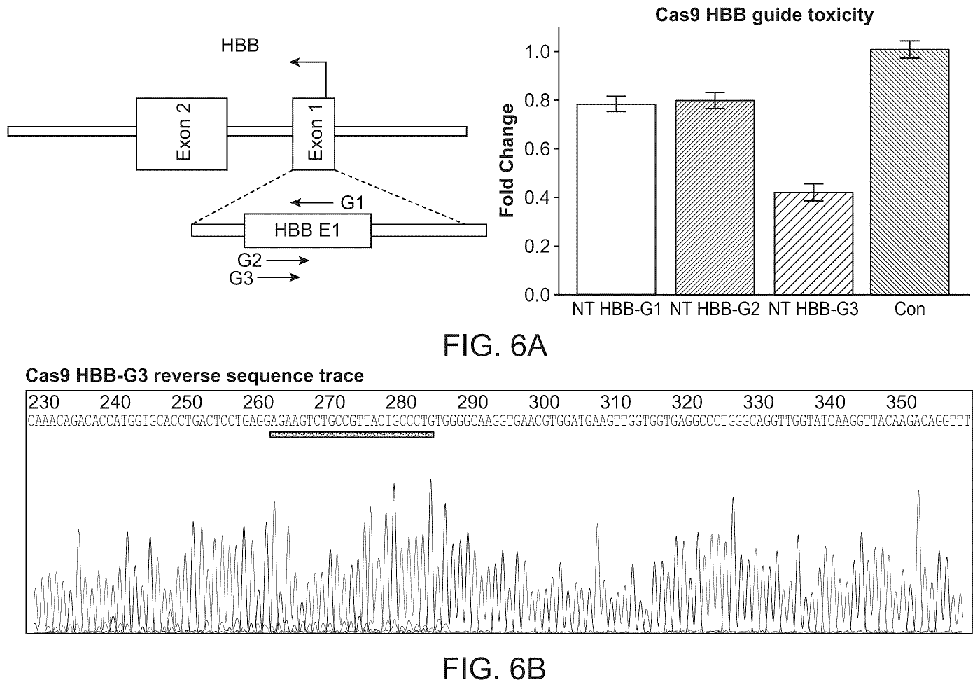

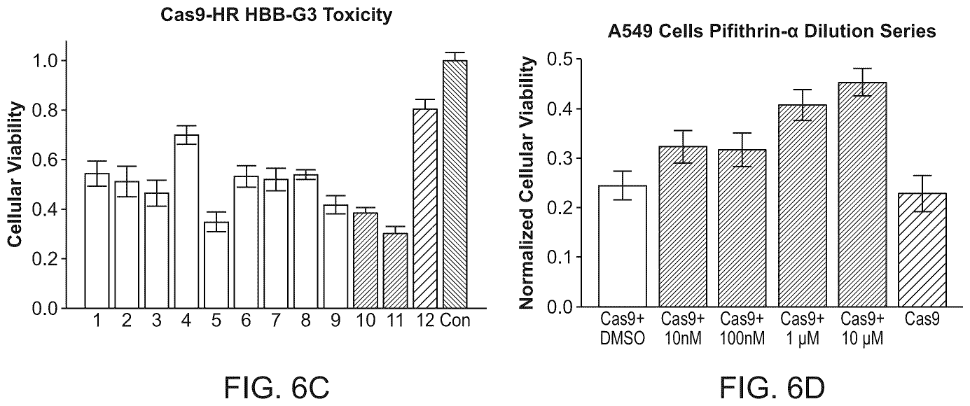

To probe whether the Cas9 mediated toxicity was due to activation of the p53 pathway, A549 cells were again transfected with Cas9-HR or Cas9 and treated with the cell permeable p53 inhibitor Pifithrin-α (Millipore Sigma). Again, after two days cellular viability was quantified via resazurin, with 10 μM Pifithrin-α treatment increasing cellular viability by ˜2-fold compared to Cas9 treated with DMSO (solvent), whereas cells transfected with Cas9-HR 8 and treated with 10 μM Pifithrin-α showed no significant change in cellular viability relative to DMSO treated cells (FIG. 1D). Additionally, treatment of A549 cells transfected with Cas9 with increasing amounts of Pifithrin-α showed a dose dependent response in cellular viability (FIG. 6D). Pifithrin-α treatment could not fully reduce Cas9 toxicity, though this could possibly be explained by incomplete inactivation of the p53 pathway. Regardless, these results indicate that the cause of Cas9 toxicity in A549 cells is at least partly due to activation of p53 mediated apoptosis, as seen in other p53+ cell types, and that Cas9-HRs do not seem to activate the p53 pathway to the same extent as Cas9, thereby offering a potential explanation for their increased cellular viability.

Next, to test the assumption that toxicity is an adequate proxy for NHEJ repair rate, three new guides were designed targeting the first exon of human beta-globin (HBB, FIG. 6A, right). Resazurin quantification of cellular viability of transfected A549 cells demonstrated that one guide polynucleotide, HBB-G3, showed significant toxicity compared to HBB-G1 or G2 (FIG. 6A, left). Sanger sequencing traces of gDNA from cells transfected with HBB-G1, G2, G3 and untransfected controls demonstrated that only HBB-G3 showed the characteristic NHEJ generated INDEL pattern (FIG. 6B), thus strongly indicating that cellular toxicity is an adequate proxy for NHEJ repair rate in A549 cells. Next, Cas9-HRs 1-9, Cas9 and Cas9+hExo1 targeting HBB-G3 were then transfected in A549 cells as before, with toxicity results shown in FIG. 6C. Interestingly, only Cas9-HRs 5 and 9 did not show a reduction in cellular toxicity, while this time Cas9-HR 4 showed the greatest reduction. These results demonstrate the importance of modulating hExo1 positioning via linker identity and length in allowing Cas9-HRs to be functional in a wide variety of genomic loci. Given the link between toxicity, NHEJ repair rate and p53 pathways it is likely that Cas9-HRs truly reduce NHEJ repair pathway activity.

### Example 3. Cas9-HRs Show Full Length Expression and Correct Subcellular Localization

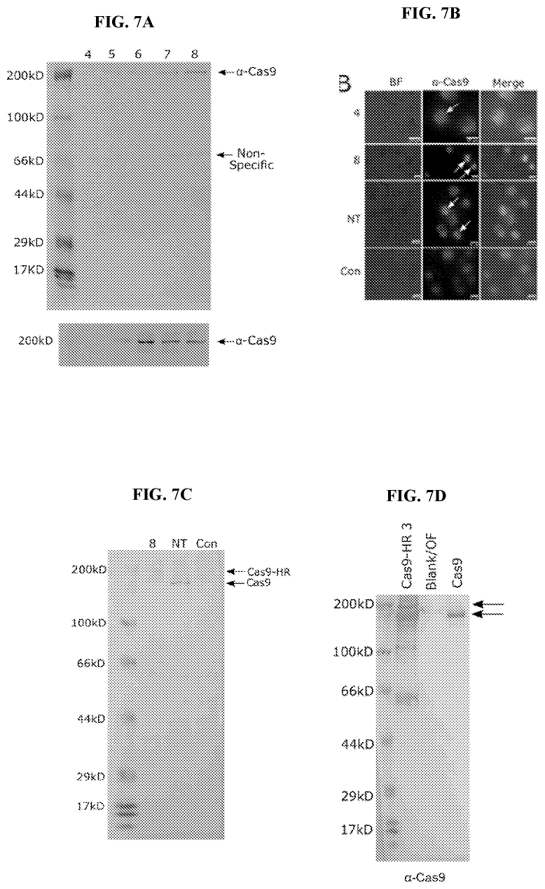

To probe Cas9-HR expression and localization, K562 cells in 24 well plates were transfected with Cas9-HRs 4-8 and Cas9 using lipofectamine 3000 (Thermofisher). Cas9-HRs 1-3 were omitted due to similar initial reduction in toxicity to Cas9-HRs 6-8 (FIG. 1D), and Cas9-HR 9 was omitted due to lack of toxicity reduction (FIG. 6C). K562 cells chosen because they are p53−/−, ideally minimizing the cellular toxicity effects of Cas9 transfection and thus facilitating accurate quantification of expression levels. K562 cells were either lysed with RIPA buffer (Santa Cruz) or fixed with 4% PFA, then probed for expression levels and sub-cellular localization via an α-Cas9 anti-body (Santa-Cruz) through both western blot and F-IHC. Detectable expression of bands at 200 kD roughly corresponding to full-length Cas9-HR (˜200-205 kD) was seen for all constructs, though with significantly reduced, though still detectable, expression levels for Cas9-HR 4 (FIG. 7A, left: top and bottom). Additionally, western blots of K562 cells transfected with Cas9-HR 8, Cas9 and untransfected control cells demonstrated the expected ˜40 kD size shift for Cas9-HR 8 compared to Cas9, and that the high molecular weight staining was specific as it was not seen in control cells (FIG. 7A, right). Finally, F-IHC experiments further demonstrated detectable expression and proper nuclear localization (white arrows) of Cas9-HRs 4-8 (data not shown for 5-7) comparable to that of Cas9 (FIG. 7B). These results indicate Cas9-HRs are properly expressed and localized when expressed in human cells, leaving only HDR activity to be assayed.

### Example 4. Cas9-HRs Simultaneously Reduce Toxicity and Increase HDR Rate

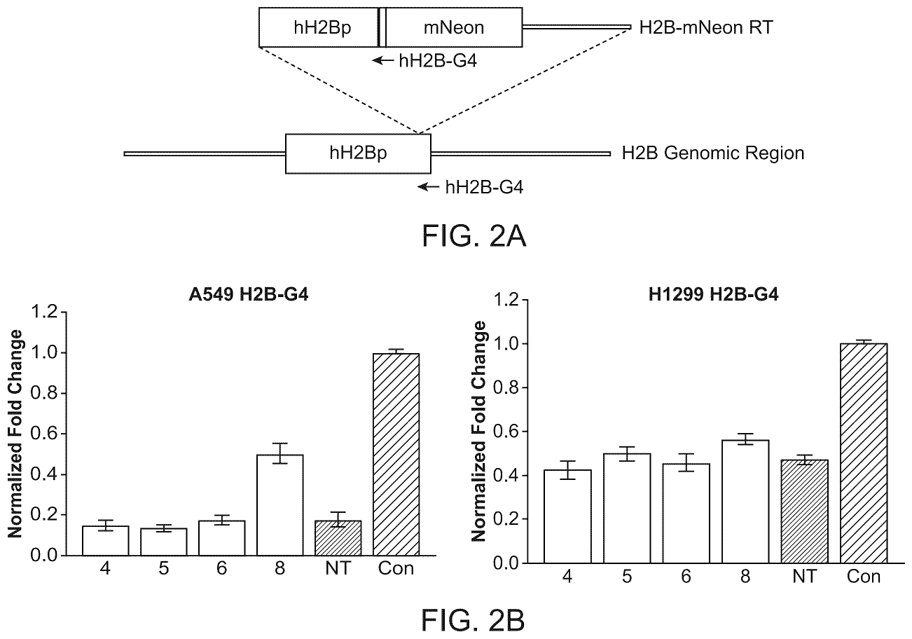

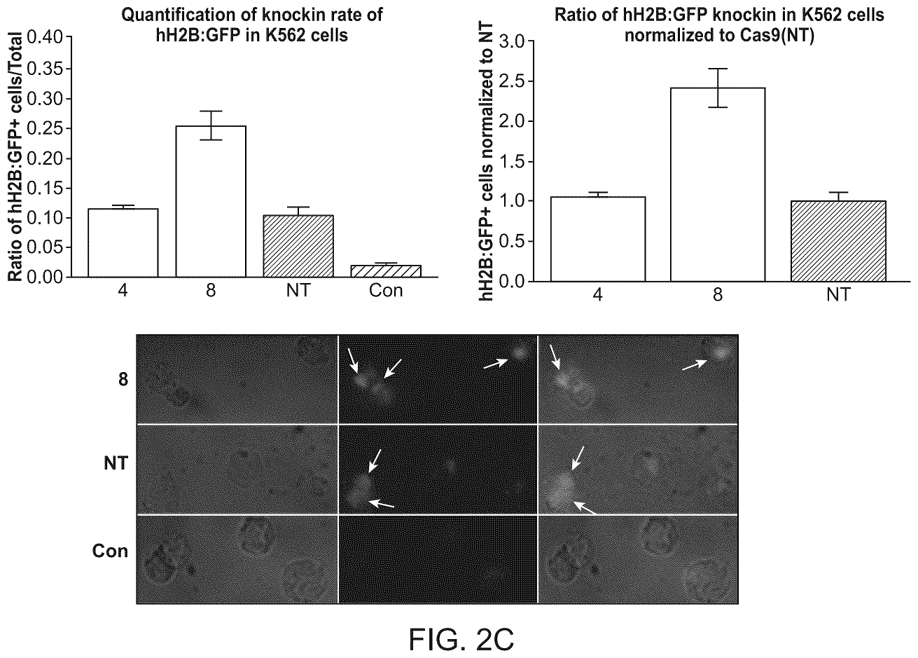

The next experiments were designed to simultaneously test both Cas9-HR nuclease activity and effects on HDR editing rate, as well as to further elucidate the role of p53 in Cas9 mediated cellular toxicity. First, a repair template designed to tag endogenous H2B with mNeon (referred to hereafter as H2B-mNeon RT) was constructed, including two silent mutations (indicated by the bar) designed to disrupt Cas9 binding (FIG. 2A). A549 cells were transfected using lipofectamine 3000 with Cas9-HRs 4,5,6,8 and Cas9 with H2B-G4 guide polynucleotides. Interestingly, Cas9-HR 8 was the only Cas9-HR to show significant reduction in cellular toxicity compared to Cas9 (FIG. 2B, left). Next, given that Pifithrin-α treatment in A549 cells likely does not fully inhibit p53 pathway activation, H1299 cells, a different lung carcinoma cell line which lacks a functional p53 gene, were used as an independent assay to further examine the effect that p53 pathway activation has on Cas9 mediated cellular toxicity. H1299 cells were plated and transfected similarly to A549 cells, and resazurin quantification of cellular viability demonstrated a dramatic reduction in toxicity for Cas9-HRs 4-6 and Cas9 in H1299 cells compared to A549, with Cas9-HR 8 only showing a small reduction (FIG. 2B, right). These results further demonstrate that Cas9-HR toxicity reduction is very likely due to reduced activation of the p53 pathway, indicating Cas9-HRs may be particularly useful in applications where significant p53 activation is undesirable.

Next, to test whether Cas9-HRs can improve HDR repair rates, cells were transfected via electroporation with both H2B-mNeon RT and only Cas9-HRs 4,8 and Cas9, since Cas9-HRs 4 and 8 had shown the most promise in reducing toxicity. After two days, cells were fixed in 4% PFA washed in PBS, then directly imaged, with cells showing nuclear localized fluorescence counted as successful HDR events. As with toxicity reduction in A549 cells, Cas9-HR 8 showed a significant increase in HDR rate compared to either Cas9-HR 4 or Cas9 (FIG. 2C, left and right). Additionally, representative fluorescent images demonstrating cells classified as HDR positive and negative cells are shown in FIG. 2C, bottom. These results demonstrate that not only do Cas9-HRs retain nuclease activity, they may in fact be able to increase the HDR rate substantially.

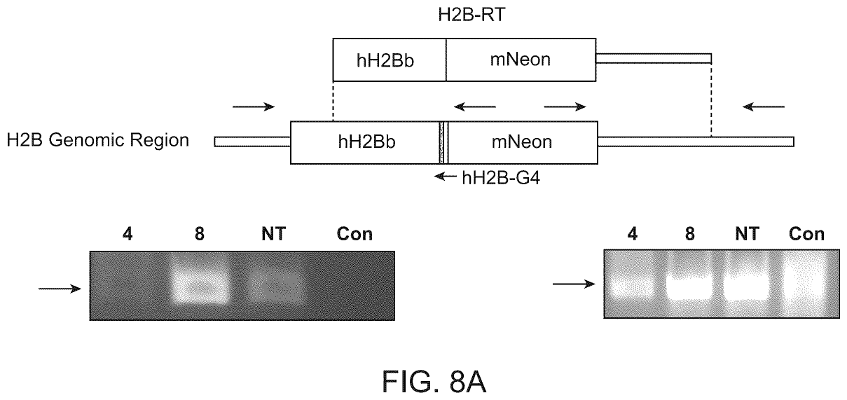

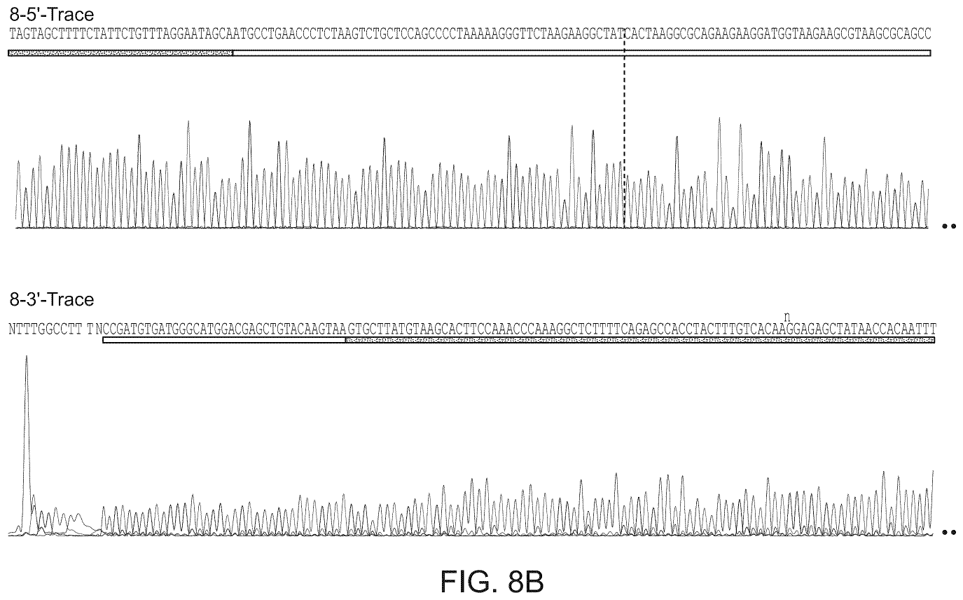

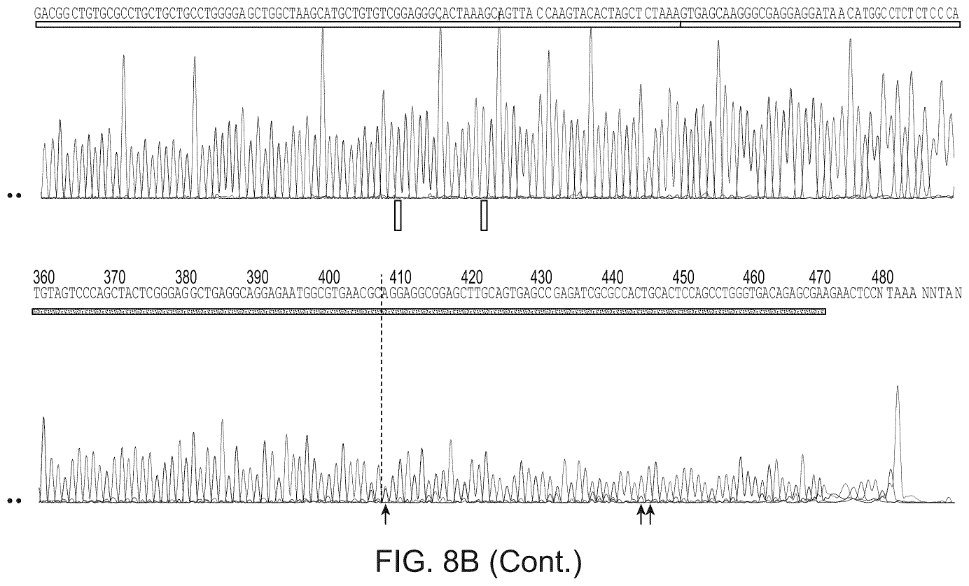

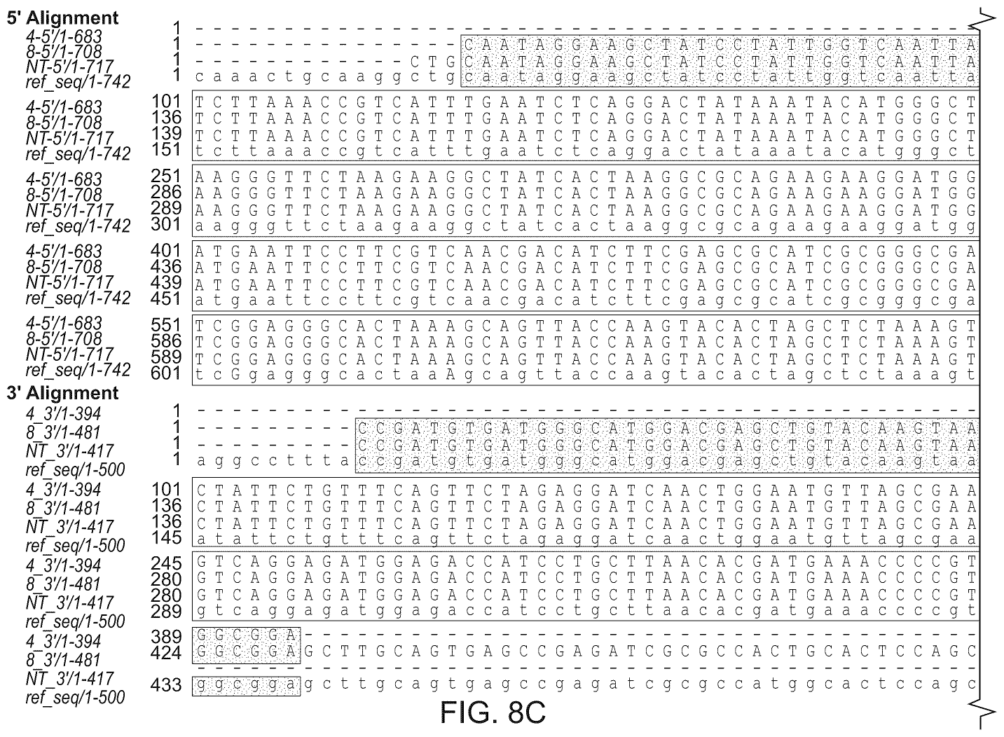

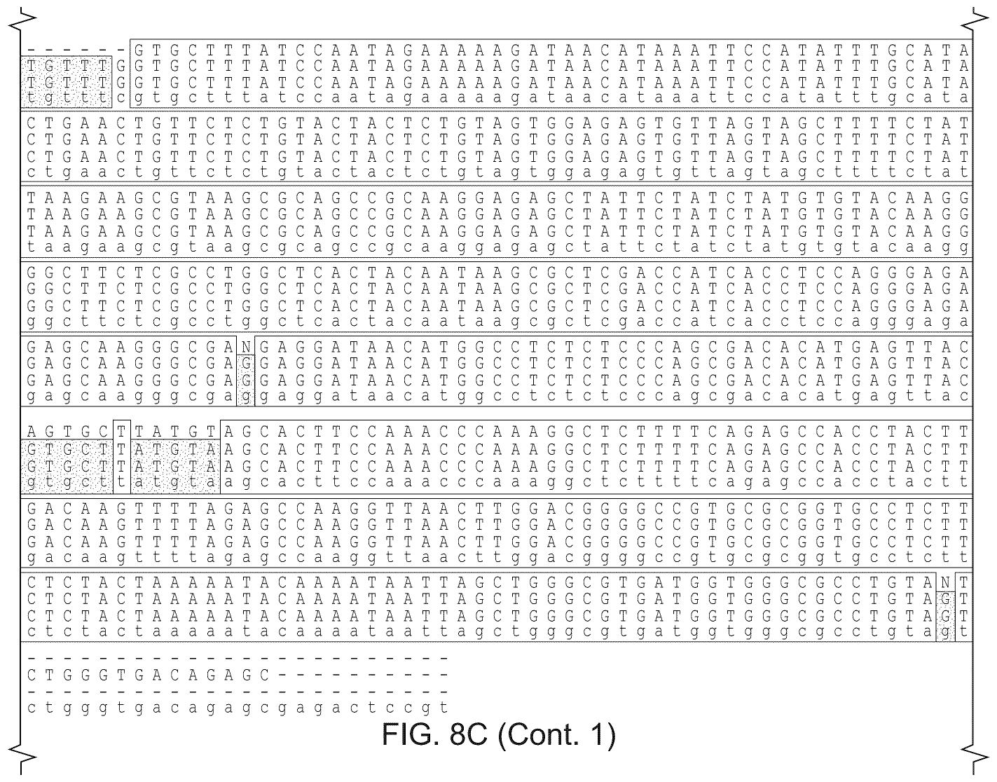

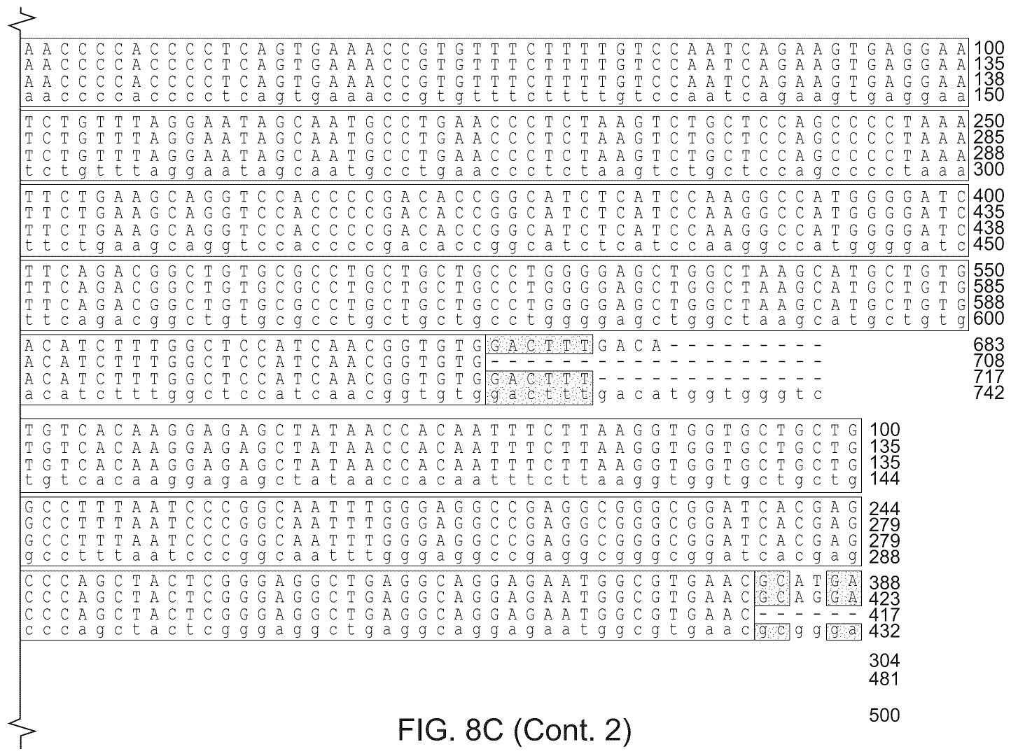

To confirm proper tagging of H2B with mNeon, DNA was extracted with the DNA-easy kit (Zymogen) from 1/10 of H2B-mNeon RT+Cas9-HR 4,8 or Cas9 transfected K562 cells. Regions surrounding the putative knock-in were amplified using 5′ and 3′ specific primers (FIG. 8A), with each primer pair containing one genome specific and one RT specific primer, ensuring amplification of only correctly integrated HDR events. Successful amplification of both 5′ and 3′ pairs was seen with both Cas9-HR 4,8 and Cas9 (NT), but not in untransfected control samples. Additionally, the increase in band strength of Cas9-HR 8 5′ product compared to Cas9-HR 4 or Cas9 further confirms the imaging results that Cas9-HR 8 likely has an increased HDR rate relative to Cas9 (FIG. 8B, 3′ appears to be out of quantitative range). The 5′ and 3′ PCR products were then gel extracted (Qiagen) and sent for sequencing (Elim Bio), where both sanger traces and consensus alignments of 4,8, and NT showed no gross differences in sequence or genomic mutations (FIG. 8C). These experiments demonstrate that Cas9-HRs not only can increase the HDR rate, but as expected based on initial design appears to maintain functionality across different cell types.

### Example 5. Cas9-HRs Decrease Toxicity and Increase HDR Rates at Multiple Additional Genomic Loci

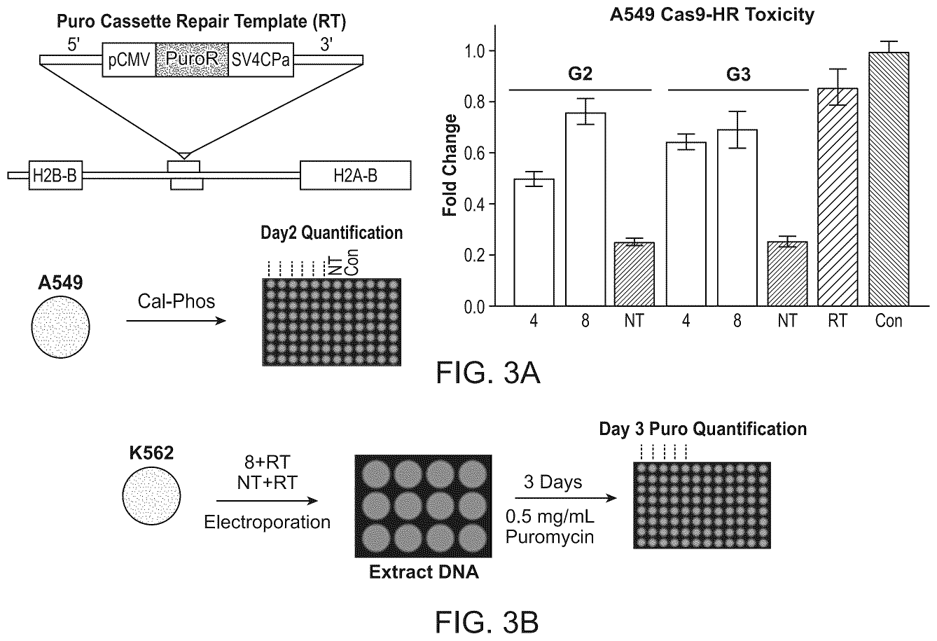

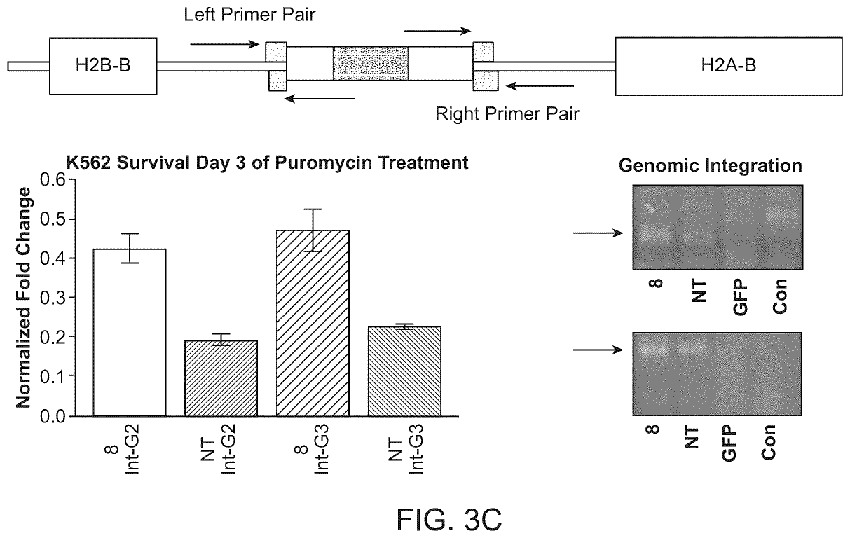

Next, an additional set of experiments were designed further test the relationship between cellular toxicity and repair pathway choice, as well as to test if Cas9-HRs can increase the rate of HDR for an independent and longer (1.8 kb) insert. First, an HDR template containing a Puromycin antibiotic resistance cassette (1.8 kb) was created via fusion PCR, with 5′ and 3′ homology arms added respectively as shown in FIG. 3A, left. The target integration site is approximately ˜1 kb to the 3′ end of the human H2B gene on Chromosome 6, an intergenic region which has no predicted genes or long non-coding RNA. Quantification of toxicity in A549 cells was again determined as in FIG. 1C. A549 were transfected via CalPhos with either Cas9-HRs 4 or 8, Cas9 targeting either Int-G2 or Int-3, or Puro RT alone. Significant toxicity was seen with Cas9 targeting both Int G-2 and G-3, while Cas9-HRs 4 and 8 both showed a dramatic reduction in cellular toxicity (FIG. 3A, right). Interestingly, Cas9-HR 4 showed significantly more toxicity targeting G-2 rather than G-3, further indicating a potential differential site preference of Cas9-HR 4 compared to Cas9-HR 8.

Next, to assay HDR rates, K562 cells were again used instead of A549 cells, as the lack of a functional p53 gene should help to deconvolute HDR rates from cellular toxicity effects. Additionally, only Cas9-HR 8 was assayed, as it was unlikely Cas9-HR 4 would be superior to Cas9-HR 8 at either locus given previous toxicity results. K562 cells were grown in 24 well plates, which were then electroporated with Cas9-HRs 8 or Cas9 and 100 ng of amplified repair template (RT), as shown in FIG. 3B. After two days, DNA was extracted from ˜ 1/10 of surviving cells and used for analysis of Puro RT genomic integration. The next day, 0.5 mg/mL puromycin was added, and after three days cellular survival was quantified via resazurin assay as described previously. As shown in FIG. 3C (left), Cas9-HR 8 G-2 and G-3 had ˜2-fold greater surviving viable cells compared to unmodified Cas9 (NT). Again, amplification using primers designed specific for the genome (containing no sequence used in the RT) and specific for the RT at both 5′ and 3′ ends demonstrated successful integration of the repair template in both Cas9-HR 8 and Cas9, but not in either GFP or untransfected cells. These results further demonstrate that Cas9-HRs have significantly increased HDR rates relative to Cas9, demonstrate cross functionality across cell types and further link reduction of toxicity to increases in HDR rates.

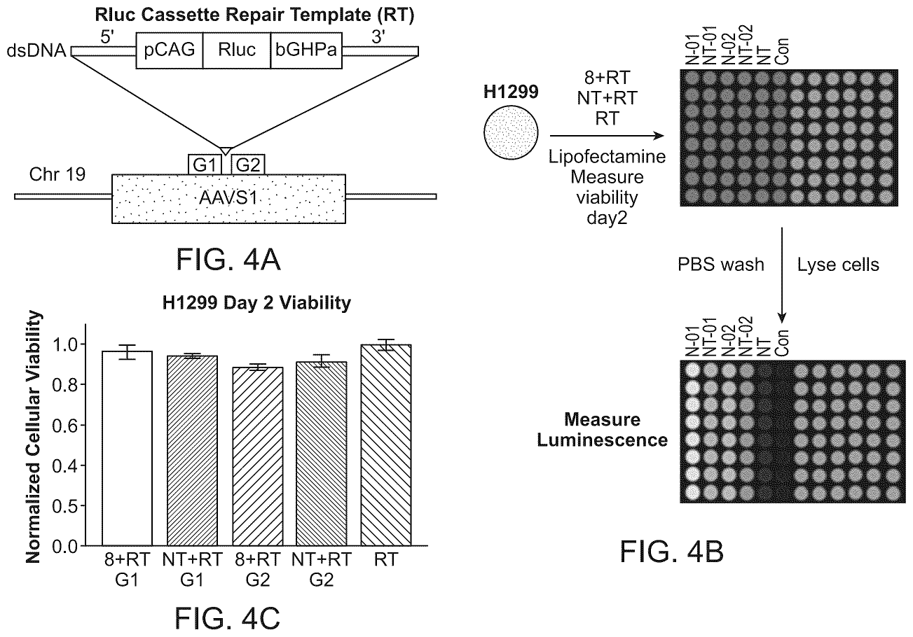

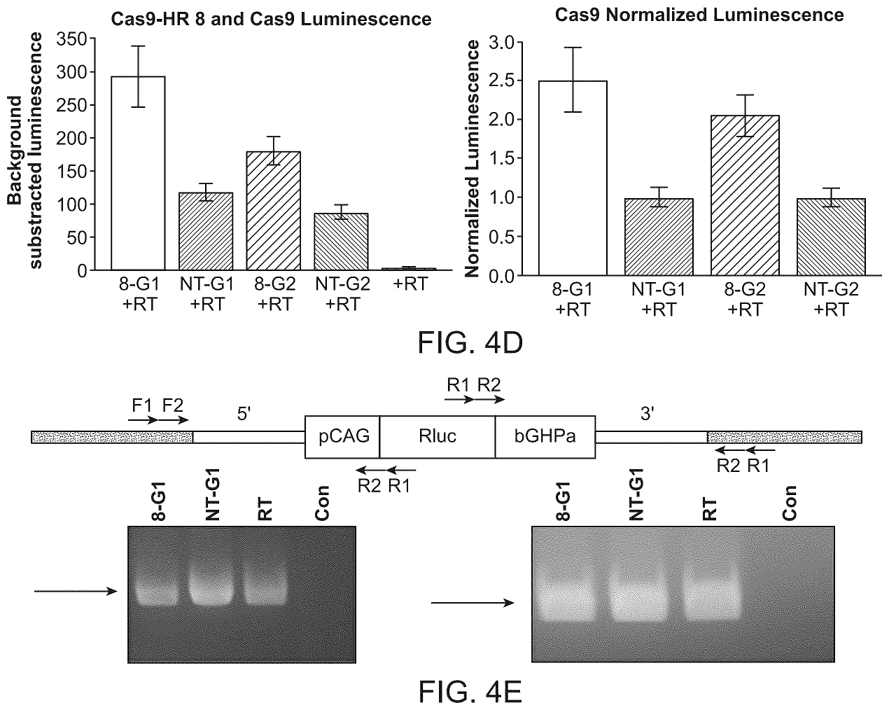

For an additional comparison of Cas9-HR vs Cas9 HDR rates, both a different integration site and HDR readout assay were used to minimize cell bias. For these experiments, the well characterized safe harbor site AAVS1 was used combined with previously established and functional guide RNAs AAVS1 T1 and T2, which will be referred to as G1 and G2 respectively hereafter. A repair template was constructed as shown in FIG. 4A, containing 5′ and 3′ AAVS1 homology arms, with a constitutively expressed Renilla Luciferase cassette (RLucRT, 2.0 kb). As with the puromycin resistance cassette, PCR amplified dsDNA was used as opposed to plasmids to avoid confounding results when quantifying HDR rates, and to avoid extra integration of plasmid specific sequences in the genome. These experiments also used H1299 cells as they are both p53−/− and adherent, which should help to minimize experimental error due to differences in Cas9-HR vs Cas9 toxicity and potential handling errors. Finally, since Cas9-HR 8 had shown the best performance with the other various targets tested to date, only Cas9-HR 8 was compared to Cas9 for these experiments.

H1299 cells were transfected with either Cas9-HR 8, Cas9 plus RLucRT targeting AAVS1 G1 or G2, RLucRT alone, or control untransfected cells (FIG. 4A). Next the viability of transfected cells was quantified via resazurin after two days, then cells were washed with PBS, then lysed and luminescence quantified via plate reader (FIG. 4B). As expected, no gross changes in cellular viability were seen with transfection of either Cas9-HR8 or Cas9 plus RLucRT compared to RLucRT alone (FIG. 4C). Next Luminescence was quantified via the Renilla-Glo Luciferase Assay System (Promega) using a 96 well plate reader with luminescence capabilities (Tecan). After data collection, raw luminescence was background subtracted from non-transfected control cells, corrected for cellular viability, and plotted in FIG. 4D. While both Cas9-HR 8 and Cas9 targeting AAVS1-G1 showed significantly higher luminescence than AAVS1-G2, Cas9-HR 8 consistently significant increases in luminescence (˜2.5 and ˜2 fold respectively) relative to Cas9 when targeted with either G1 or G2 (FIG. 4D, left, right). As before, integration in the genome was confirmed via junction PCR, with successful amplification for both 5′ and 3′ junctions with successful amplification seen with all treatments except controls (FIG. 4E). Given the extremely sensitive nature of nested PCR, it is not surprising that integrations were detected in the RT samples, even if the amount were too low to be detected via luminescence assay. Sequencing of purified amplicons from each treatment confirmed correct and specific amplifications, with no gross abnormalities seen (data not shown). Combined with previous results, this data strongly indicates that the Cas9-HR series (and Cas9-HR 8 in particular) has significantly higher HDR editing rates than Cas9 and is functional across multiple cell types and genomic loci.

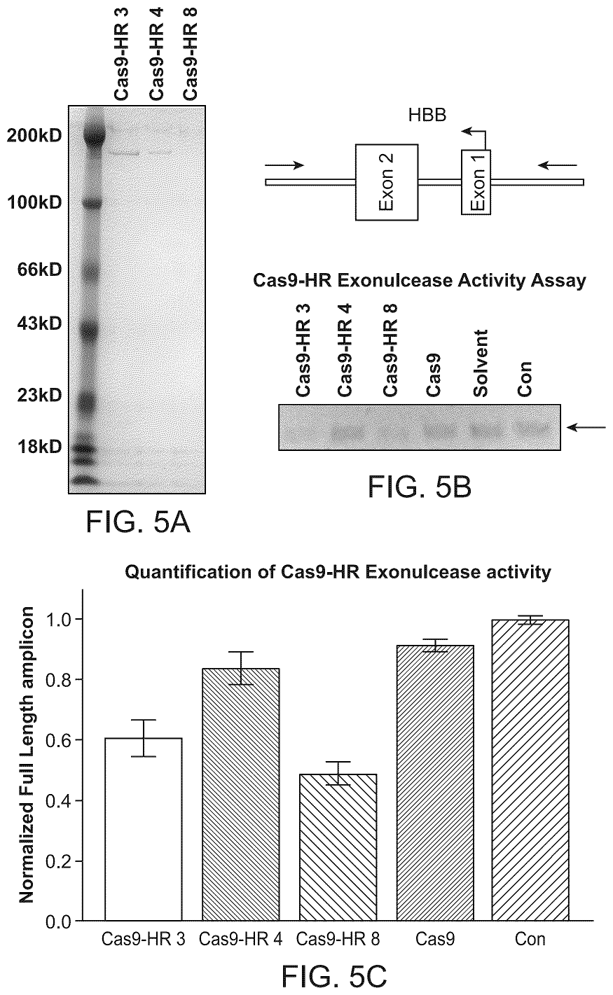

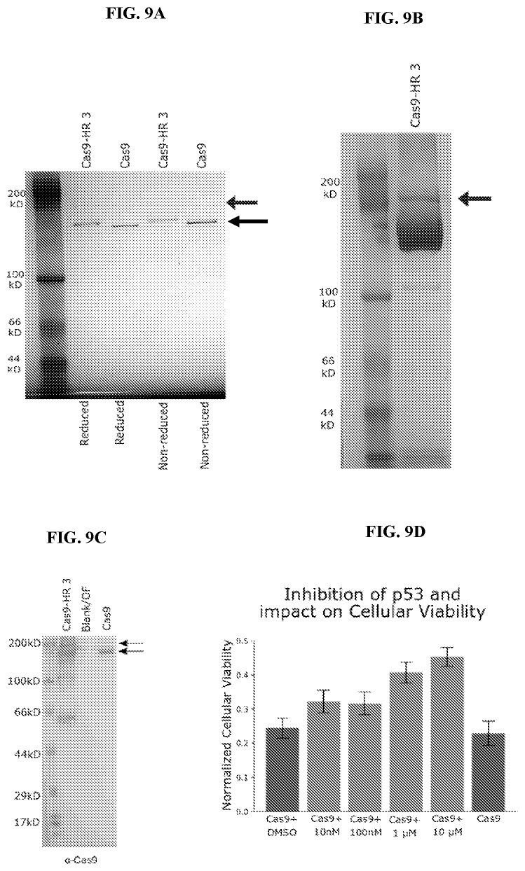

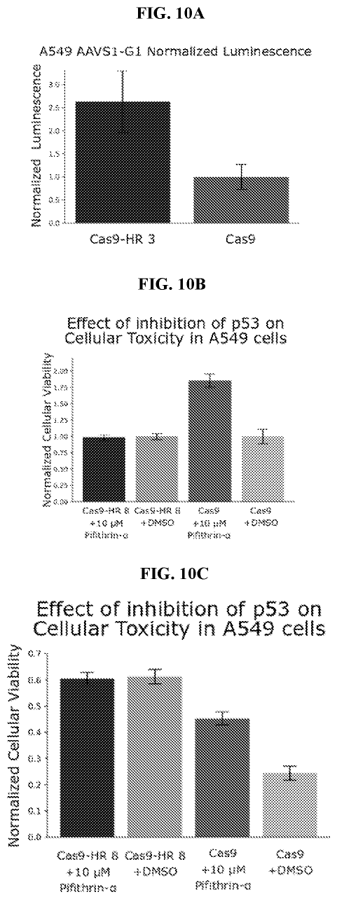

Finally, given the increasing popularity of RNP applications, protocols for bacterial expression and purification of Cas9-HRs 3, 4, 8 were developed. As RNP preparations of Cas9 generally lack the N-terminal NLS, Cas9-HR 3 was included to test if the N-terminal NLS would interfere with Cas9-HR 8 purification, and Cas9-HR 4 due to potential differential site preference. Successful purification of Cas9-HR 3, 4 and 8 is shown with a representative SDS-PAGE gel (FIG. 5A), though it appeared that the majority of Cas9-HRs 3 and 4 are cleaved (FIG. 9A, B). Cleavage was confirmed via western blot against Cas9, which confirmed the (˜200 kD) band is full length Cas9-HR 3, while the smaller products seen in FIG. 9A and FIG. 9B are digestion products co-purified with full length Cas9-HR 3 (FIG. 9C). FIG. 9D illustrates the impact on A549 cell viability when p53 was inhibited. A549 cells were plated in 96 well plates, transfected with Cas9 (NT) targeting the intergenic region on Chromosome 6, and treated with either DMSO or increasing concentrations of Pifithrin-α. FIG. 10A illustrates SDS-PAGE gel of purified Cas9-HRs 3, 4, and 8. Cas9-HRs 3 and 4 may undergo some proteolytic cleavage during expression/purification, however all three have upper bands which run at the predicted full-length size (˜200 kD) of Cas9-HR.

FIG. 10B illustrates exonuclease activity assays for purified Cas9-HRs. The top diagram shows the HBB genomic region, and the location of primers used to amplify the amplicon used in following nuclease assays. 30 nM of Cas9-HRs 3, 4, 8 and Cas9 and control reactions were incubated with 3 nM of the purified HBB amplicon, and incubated at 37° C. for 60 minutes, after which 1 μL of proteinase K (200 μg/mL) was added and reactions incubated at 65° C. for an additional 20 minutes. Reactions were then electrophoresed and visualized on an agarose gel.

FIG. 10C illustrates quantification of exonuclease activity. Quantification of exonuclease activity assayed in FIG. 10B. 3 replicates were performed for each reaction and were quantified via FIJI (ImageJ) and then normalized to control amplicon levels. Cas9-HRs 3 and 8 show significant exonuclease activity, whereas neither Cas9-HR 4 or Cas9 show no significant activity. During purification it was noted that Cas9-HR 4 required somewhat different conditions for successful purification, and may require different reaction conditions than Cas9-HRs 3 and 8.

Dose dependent increases in cellular viability can be seen with increasing concentrations of Pifithrin-α, with 10 μM showing a statistically significant (p<0.001) increase in viability compared to DMSO or Cas9 only treated cells (students t-test, two-sided). Even with significant cleavage in Cas9-HRs 3 and 4, exonuclease activity assays were performed using an amplified region surrounding Exon1 of HBB (FIG. 5B) as a simple first pass to test for catalytic activity of purified Cas9-HRs. Both Cas9-HR 3 and Cas9-HR 8 showed a significant reduction in the full length HBB amplicon compared to the other treatment conditions and controls (FIG. 5C). Though it was somewhat surprising that Cas9-HR 4 did not show Exonuclease activity, it was noted throughout the purification protocol that Cas9-HR 4 required different binding and elution conditions, and might require a different buffer composition than Cas9-HR 3 and 8. Though significant optimization remains in order to produce majority full length protein, successful purification of soluble full length and active Cas9-HRs is a promising first step in extending Cas9 fusion protein based HDR improvement to RNP based methods.

### Example 6. Materials and Methods for Experimentations in Examples 1-5

### Genomic DNA Extraction, Amplification and Sequencing

DNA was extracted from cells using the DNA-easy mini kit (Zymogen) per manufacturer's instructions. DNA was then amplified with either standard Taq (Bioneer), Fusion (NEB), Q5 (NEB), or PrimeSTAR GXL (Takara) using standard PCR protocols. Genome integrated H2B-RT-3′ was amplified with standard taq and primers hH2B-3′-F and -R with Tm=56° C. for 35 cycles; H2B-RT-5′ was amplified using PrimeSTAR GXL and primers hH2B-5′-F and -R at Tm=65° C. for 35 cycles. Genome integrated PuroRT-3′ was amplified with Phusion polymerase and Puro-Int-3′-F and -R at Tm=58° C. for 35 cycles; PuroRT-5′ was amplified with Phusion polymerase and Puro-Int-5′-F and -R at Tm=56° C. for 35 cycles. Genome integrated RLucRT was amplified using nested PCR with Primestar GXL. The first round was performed using Rluc_G-5′-F2 and Rluc_G-5′-R2 for the 5′ junction and RLuc_G-3′-F1 and Rluc_G-3′-R for the 3′ junction, Tm=64° C. for 30 cycles. Reactions were then diluted 1:200 and the second round of PCR was performed for 5′ with Rluc_G-5′-F1 and avRT-rLuc-P2-R, and for 3′ with Rluc_G-3′-F3 and Rluc_G-3′-R2, Tm=62° C. for 30 cycles. All sequencing was carried out gel extracted amplicons (Qiagen), and sanger sequenced using primers used for amplification. Sequences were inspected and visualized with ApE and Jalview. SEQ ID NOs: 121-153 and 161-191 illustrate exemplary nucleic acid sequences of primers for the amplification and sequencing.

### Cell Culture and Transfection and Drug Delivery:

Unless otherwise noted, adherent cells (A549 or H1299) were seeded in 96 well plates and grown in either 50/50 F-12/DMEM or RPMI-1640 respectively, supplemented with 5% FBS and grown to roughly 70% confluency. Cells were then transfected with either Cal-Phos as described in Chen 2012 or Lipofectamine 3000 (Thermofisher). Fresh media was exchanged the next day and cellular viability was quantified on day 2.

K562 were grown in 24 well plates with RPMI-1640 supplemented with 5% FBS until roughly 70% confluent. At that time cells were either electroporated using the mNeon system (Invitrogen) using optimized settings for K562 cells, or lipofectamine 3000 again following manufactures instructions. Cells were then returned to 24 well plates with fresh media and grown for at least two days before being used in downstream analysis.

Pifithrin-α (Millipore sigma) was diluted to appropriate concentrations such that a 1:100 dilution resulted in the desired final concentration and was applied concurrent with transfection, with equal amounts of DMSO added as a control.

### Cellular Toxicity Quantification

20 μL of 0.15 mg/mL Resazurin solution was added to cells either plated in 96 well plates for all cell lines, which were then incubated for ˜4 hours at 37° C. Cellular toxicity was quantified using a Tecan SpectraFlour Plus plate reader (Tecan) using a 535/595 filter set. Raw data was then normalized to the mean of the untransfected controls and plotted as described.

### H2B-mNeon Knock-In HDR Quantification

K562 cells were transfected with 500 ng of Cas9-HR 4,5,6,8 or Cas9 targeting hH2B-G4, plus 50 ng of hH2B-mNeon RT (SEQ ID NO: 201). After 2 days, cells attached to coverslips coated with 0.01% poly-1-lysine, and were fixed in 4% PFA (Thermofisher) for 15 minutes at RT. After fixation, cells were washed 3× in PBS, mounted in 50% glycerol and imaged on a Nikon Eclipse E600 with standard FITC filters. For quantification, random patches of cells (usually between 8-15 cells per image) were identified via Brightfield, then fluoresce images were taken with constant illumination and exposure time (100%, and 110 ms respectively). After acquiring roughly 10 images per construct, ratios of positive to total cells were calculated and plotted either in absolute or normalized to Cas9 (NT), with two independent experiments each treatment.

### Cas9-HR PuroRT HDR Quantification

K562 cells were transfected with 500 ng of Cas9-HR 8 or Cas9 targeting Int-G2 or Int-G3, plus 50 ng of Puro RT (SEQ ID NO: 202). After two days, 1/10 of cells were taken for DNA extraction, while the rest were treated with 0.5 μg/mL puromycin. Cells were grown for a further 3 days then viability quantified via Resazurin assay.

### Cas9-HR and Cas9 Luminesce Assays

H1299 were seeded and grown in 96 well plates as for other experiments, then transfected with 500 ng of Cas9-HR 8 or Cas9 (NT) targeting either AAAVS1 G1 or G2 with 50 ng of AAVS1 RLucRT (SEQ ID NO: 203). After two days, cellular viability was quantified via Resazurin, then washed with PBS and lysed with 25 μL of cell lysis buffer, and luciferase activity was quantified via Renilla Luciferase Assay (Promega) and using a Tecan Infinite Mplex plate reader.

### Cas9-HR and Cas9 Protein Purification

pET-28b-Cas9-HR 3,4, 8 and pET-28b-Cas9 were transformed into BL21(DE3) bacteria. Single colonies were picked and grown overnight at 37° C. in LB supplemented with 75 μg/mL Carbenicillin. The next day, each was diluted was 1:100 in fresh Terrific Broth media supplemented with 75 μg/mL Carbenicillin, 0.05% Glucose, 10-50 μM IPTG and grown overnight at room temperature. Purification protocols were based on a modified version of a previously published two-step Cas9 purification protocol.

### Cas9-HR and Cas9 In-Vitro Exonuclease Assay

DNA was isolated from wildtype K562 cells, and amplified using standard Taq DNA polymerase and HBB-out-F (5′-aacgatcctgagacttccaca-3′ (SEQ ID NO: 125)) and HBB-out-R (5′-tgcttaccaagctgtgattcc-3′ (SEQ ID NO: 126)), Tm=56 for 35 cycles, and purified using the Qiagen PCR cleanup kit. Cas9-HR 3,4,8 or Cas9 were combined with the amplified HBB fragment at a 10:1 molar ratio (30 nM: 3 nM) in 1× Cas9 reaction Buffer (50 mM Tris, 100 mM NaCl, 10 mM MgCl₂, 1 mM DTT, pH7.9) and incubated for 1 hr at 37° C., after which 1 μL of Proteinase K (NEB) was added and the reaction was incubated for an additional 20 minutes at 65° C. The samples were then electrophoresed on a standard 1% TAE agarose gel stained with gel green.

### Cloning

pX330 was digested with AgeI, EcoRI and EcoRV, then electrophoresed and gel purified using standard procedures (Qiagen). Cas9-HR fusions 1-9 were created by amplifying hExo1 (1-352) and Cas9 from pTXB1 and pX330 respectively. Fragments were electrophoresed and gel purified using standard procedures, then stitched together using the following fusion PCR protocol: equimolar amounts of both fragments without primers were run for 10 cycles at a Tm of 58° C., after which outer primers were added and reaction continued for 20 cycles at a Tm of 62° C. Fragments were then electrophoresed, and correct sized fragments gel purified as before. Cas9-HR 1-9 purified fragments were then cloned into the pX330 backbone using infusion cloning (Takara). After colony picking and sequencing to confirm no mutations were present, Cas9-HRs 1-9 plasmids were purified using zymopure miniprep kit (Zymogen). Cas9-HRs 1-9 were then digested with BbsI, and gel purified as before. Guides (e.g., guide polynucleotides comprising nucleic acid sequences of any one of SEQ ID NOs: 101-117) were cloned in using standard protocols briefly consisting of denaturation and subsequent annealing, phosphorylation of 5′ ends using PNK, then final cloning using T4 ligase. Colonies were picked and screened via PCR to ensure correct cloning of guide polynucleotides. Cas9-HRs 1-9 with various guide polynucleotides were then purified via ZymoPURE miniprep kits (Zymogen).

pET-28b Cas9-HRs 3,4,8 were created by synthesizing new E. Coli codon optimized hExo1 fragments including linkers. pET-28b Cas9 was digested with NcoI, then the backbone was gel purified as before. Both hExo1 3,4,8 and a N-terminal fragment of pET-28b were amplified using Phusion polymerase. Fragments were then purified, then stitched together using a similar fusion PCR protocol as above: 10 cycles at Tm 62° C. without primers, then 20 cycles at Tm 62° C. Correct sized fragments were then gel purified, then cloned into NcoI digested pET-28b using infusion as before.

### Western Blotting

Total protein was extracted from K562 cells transfected with 500 ng of Cas9-HRs 4-8 or Cas9 using RIPA buffer supplemented with 2 mM PMSF, 1 mM Sodium Orthovandate, and 1 mM protease cocktail inhibitor (Santa Cruz). After quantifying protein concentration via Bradford Assay (BioRad), 5 μg total protein was run on a NuPage 4-12% Bis-Tris precast Gel (Thermofisher), then transferred at 30V for 1 hr to a nitrocellulose membrane (Sigma) using the X-cell II blot module (Invitrogen). The membrane was the washed 2-4× for 5 minutes with PBST, blocked for 30 minutes with 5% non-fat milk, washed 2× with PBST, then incubated with α-Cas9 (1:1000, Santa Cruz), for 1 hr at room temperature, then overnight at 4° C. After washing 4-6× for ˜ 10 minutes each wash with PBST, the membrane was incubated with α-mouse-HRP (1:1000, Santa Cruz) for 1 hr at RT, and overnight at 4° C. After washing 2-4× for 5 minutes per wash with PBST, the membrane was incubated with a 1× NC/DAB (Thermofisher) solution for 15-30 minutes after which the gel was imaged.

### F-IHC

K562 cells transfected with 500 ng of either Cas9-HR 4-8 or Cas9, were attached to coverslips coated with 0.01% Poly-1-lysine and fixed with 4% PFA (Thermofisher) for 15 minutes at RT and washed 4× for 5 minutes with PBST. Cells were blocked with 5% BSA for 30 minutes, then incubated with α-Cas9 (1:1000, Santa Cruz) for 1 hr at RT, then 4° C. overnight. The next day cells were washed 4× with PBST for 5 minutes per wash, then incubated with m-IgGₖ BP-CFL 488 (1:1000, Santa Cruz) for 1 hour at RT, then overnight at 4° C. After an additional 4×PBST washes of 5 minutes per wash, cells were mounted in 50% glycerol, then imaged using a Nikon Eclipse E600 and standard FITC filters.

### Sequence Analysis

A Plasmid Editor (APE, Wayne Davis) was used for all sequence analysis and to generate images of sequence traces. Alignment of sequencing results the 5′ and 3′ PCR product from H2B-mNeon Cas9-HRs 4,8, and Cas9 with the reference sequence. Sequences were aligned using ClustalOmega and pseudo-colored using Jalview to show percent identity of all sequences.

### Example 7

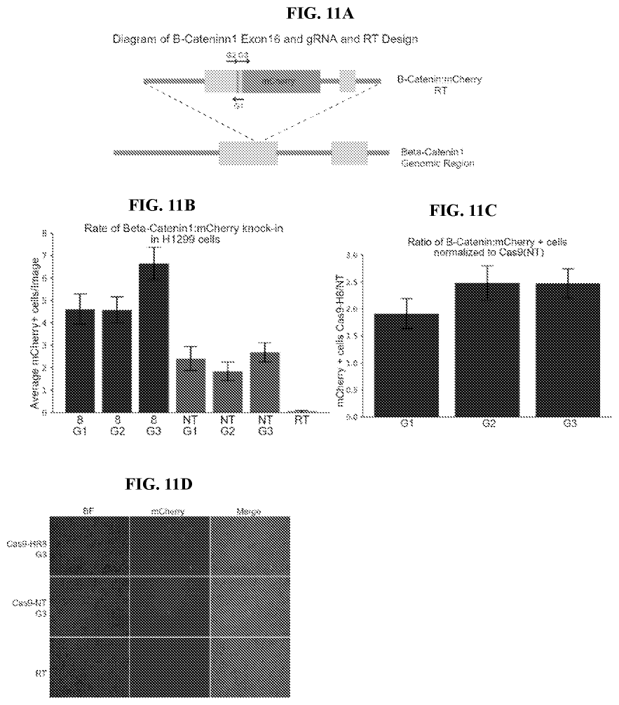

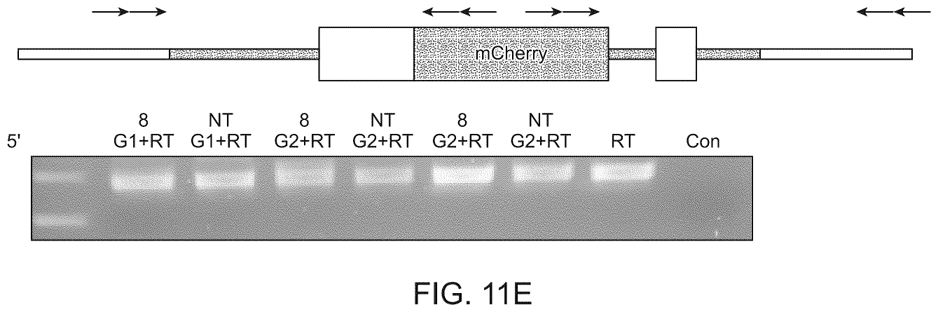

### Example 7. Methods for Experimentation of FIGS. 11-17

### FIGS. 11A-E

H1299 cells were seeded in 24 well plates, grown to about 70% confluency, then transfected using lipofectamine 3000 with 250 ng of pCAG-Cas9-HR8 or pCAG-Cas9, 250 ng of pU6-Beta Catenin-G1, -G2, or -G3, and 50 ng of Beta-Catenin1:mCherry RT. After three days, media was replaced with HBSS (to reduce background for imaging) imaged using an EVOS M5000 imaging system. Imaging was performed by randomly finding and focusing on at least ten different sections per well using a 20× magnification lens, after which both brightfield and RFP images were acquired. Imaged were quantified by hand, using control images as the basis for RFP/mCherry background fluorescence. Stats: All two-sided students T-test with unequal variance: [Cas9-HR8, G1] vs [Cas9, G1] p<0.01 (0.00922); [Cas9-HR8, G2] vs [Cas9, G2] p<0.001 (0.0005); [Cas9-HR8, G3] vs [Cas9, G3] p<0.0001 (9.595E-05); [Cas9-HR8, G3] vs [Cas9-HR8, G1] p<0.05 (0.046); [Cas9-HR8, G3] vs [Cas9-HR8, G2] p<0.05 (0.031); all Cas9-HR8 and Cas9 vs RT p<<0.0001; and other relations, p>0.05. SEQ ID NOs: 110-117 provide examples of guide polynucleotides targeting Cadherin (Ecad), Beta Catenin (B-Catenin1), and Safe Harbor Site 231 (SHS-231) for inducing the HDR by the Cas9-HR described herein.

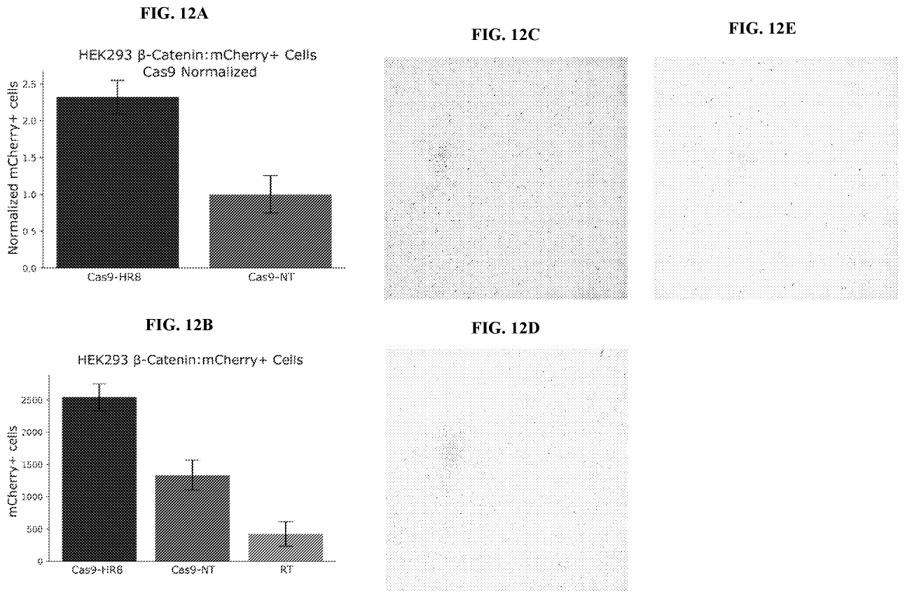

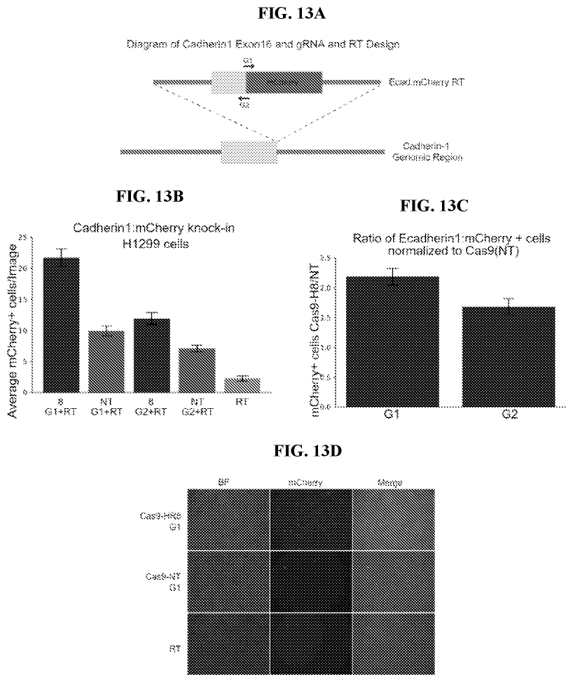

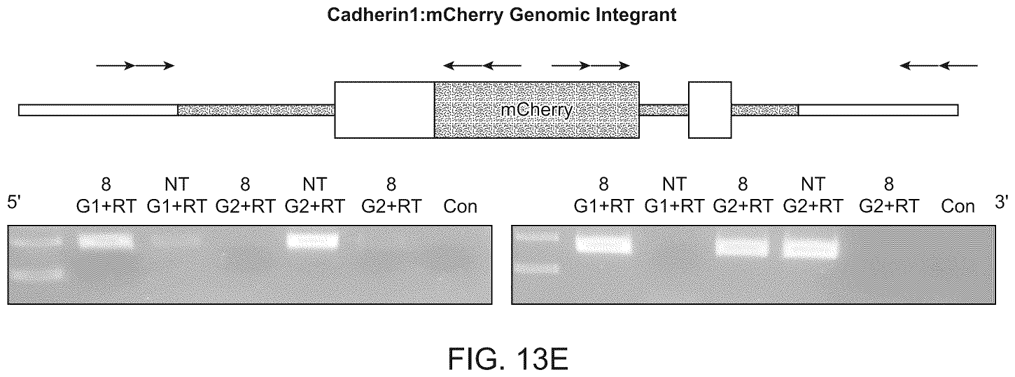

### FIGS. 13A-E

H1299 cells were seeded in 24 well plates, grown to about 70% confluency, then transfected using lipofectamine 3000 with 250 ng of pCAG-Cas9-HR8 or pCAG-Cas9, 250 ng of pU6-Ecad-G1, and 50 ng of Cadherin1:mCherry RT (˜750 bp). After three days, media was replaced with HBSS (to reduce background for imaging) imaged using an EVOS M5000 imaging system. Imaging was performed by randomly finding and focusing on at least ten different sections per well using a 10× magnification lens, after which both brightfield and RFP images were acquired. Imaged were quantified by hand, using control images as the basis for RFP/mCherry background fluorescence. Stats: All two-sided students T-test with unequal variance: [Cas9-HR8, G1] vs [Cas9, G1] p<10\hat{ }-6 (1.8E-07); [Cas9-HR8, G2] vs [Cas9, G2] p<0.0001 (1E-04); [Cas9-HR8, G1] vs [Cas9-HR8, G2] p<0.05 (3.9E-06); [Cas9, G1] vs [Cas9, G2] p>0.05 (0.10575); all Cas9-HR8 and Cas9 vs RT p<<0.0001; and others, p>0.05

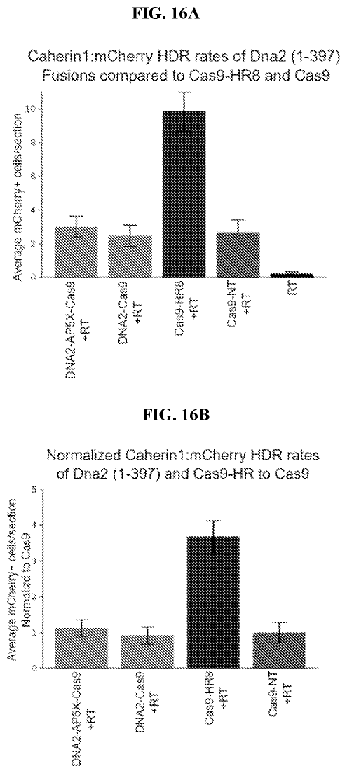

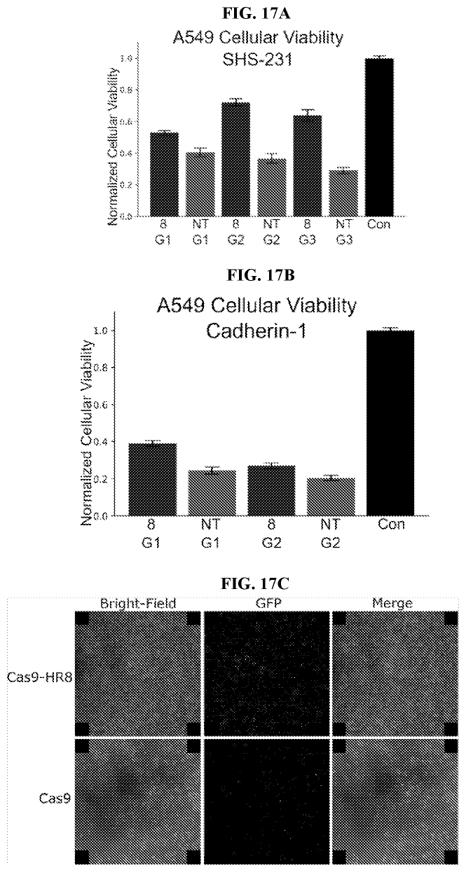

### FIG. 17A and FIG. 17B

20 μL of 0.15 mg/mL Resazurin solution was added to cells either plated in 96 well plates for all cell lines, which were then incubated for ˜4 hours at 37° C. Cellular toxicity was quantified using a Tecan SpectraFlour Plus plate reader (Tecan) using a 535/595 filter set. Raw data was then normalized to the mean of the untransfected controls and plotted as described. Guide polynucleotides used and plasmids used for the resazurin assay: pCAG-Cas9-HR or pCAG-Cas9; pU6-SHS-G1, G2, G3 and pU6-Ecad-G1 and G2. Stats: All two-sided students T-test with unequal variance. SHS-231: [Cas9-HR8, G1] vs [Cas9, G1] p<0.005 (0.002109); [Cas9-HR8, G2] vs [Cas9, G2] p<<0.0001 (2.90048E-07); [Cas9-HR8, G3] vs [Cas9, G3] p<<0.0001 (3.46688E-06); and all p<<0.001 compared to controls. Cadherin1: [Cas9-HR8, G1] vs [Cas9, G1] p<0.001 (0.000243); [Cas9-HR8, G2] vs [Cas9, G2] p<0.05 (0.011678); and all p<<0.001 compared to controls

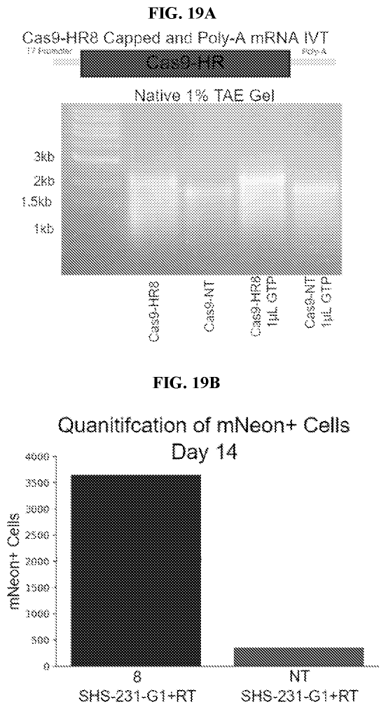

### FIG. 18B, and FIG. 19B

H1299 cells were seeded in 96 well plates, grown to ˜70% confluency, then transfected with 500 ng of pCAG-Cas9-HR8 or pCAG-Cas9, 500 ng of pU6-SHS-G1, and 50 ng of SHS-231-pCAG-mNeon RT per each column of 8 wells. After reaching confluency, cells were tryspinized and seeded in 6 well plates, which were then imaged at 10× magnification using a Leica THUNDER Epifluorescent microscope. After imaging, thresholds and ROIs for mNeon+ cells were generated using FIJI (ImageJ). Quantification of mNeon+ cells was done via simple count of ROIs for Cas9-HR8 or Cas9 treatment, boxplots were generated using a custom script using python. Stats: average expression value [Cas9-HR8, G1] vs [Cas9, G1] p<<<0.0001 (p=0).

### FIG. 19A

Cas9-HR and Cas9 CDS were cloned into vectors containing strong T7 promoters, 5′ and 3′ UTR from human B-Globin. An approximately 150 base-pair long poly-A tail was then added via PCR, with ˜-150 ng of the resulting product used as template for a 5′ capped in-vitro transcription reaction using the mMessage Machine T7 transcription kit (Thermo). 20 μL reactions were incubated for 90 minutes at 37° C. for 90 minutes, after which 1 μL of DNaseI was added and incubated for an additional 15 minutes at 37° C.

### Example 8. Increased Insertion of Repair Template Encoding Beta-Catenin Induced by Cas Fusion Protein Complex

HEK293T cells were seeded in 24 well plates, grown to ˜70% confluency, then transfected using lipofectamine 3000 with 250 ng of Cas9-HR8 or Cas9, 250 ng of Beta-Catenin-G3, and 50 ng of Beta-Catenin1:mCherry RT (˜750 bp). After three days, media was replaced with HBSS (to reduce background for imaging) imaged using an Cytation5 imaging system. Imaging was performed by taking a 6×6 stitched image in the middle of the well using an 10× magnification lens, where both brightfield and RFP images were acquired. HDR rates were quantified via ImageJ, two independent replicates were performed per treatment. FIG. 11A illustrates an exemplary Beta-Catenin1:mCherry RT design diagram showing the human genomic region surrounding the last exon (exon 16) of Beta-Catenin1. Three different gRNAs are denoted by black arrows (G1, G2, G3), with arrow direction indicating the targeted strand. A repair template was constructed containing approximately 750 bp long 5′ and 3′ homology arms, exon 16 of Beta-Catenin1, silent mutations introduced to prevent gRNA binding to the RT, and a direct fusion of the fluorescent protein mCherry (˜750 bp). FIG. 11B illustrates quantification of Beta-Catenin1:mCherry HDR rate in H1299 cells showing the average mCherry+ cells per image section (>10 for all conditions) imaged at day 3 post transfection. Both Cas9-HR8 and Cas9 showed significant increases in positive cell count compared to Beta-Catenin1:mCherry alone. Compared to Cas9, Cas9-HR8 showed significant increases in mCherry+ cells compared to Cas9 for all three gRNAs. When comparing gRNA performance within Cas9-HR8 or Cas9 treated cells, G3 showed the highest HDR efficiency and similar patterns of increases for both Cas9-HR8 and Cas9, indicating Cas9-HR8 augmented HDR efficiency, rather than fundamentally changing the behavior of Cas9. FIG. 11C illustrates relative fold increase in Beta-Catenin1:mCherry HDR (Cas9-HR8 normalized to Cas9) showing the normalized fold change of Cas9-HR compared the corresponding guide polynucleotide for Cas9 (e.g., Cas9-HR8 G1/Cas9 G1). All guide polynucleotides showed significant (>2 fold) increases in mCherry+ cells relative to Cas9, with G2 and G3 showing the highest (˜2.5) fold change. FIG. 11D illustrates representative images of Beta-Catenin1:mCherry+ Cells showing images from Bright Field (BF), mCherry (for increased visibility of cells) and a merge of the two for Cas9-HR8 and Cas9 G3, and RT only. Beta-Catenin1 is primarily localized to the membrane, however, can localize to the nucleus upon Wnt pathway activation. The expected membrane localization of mCherry (and possible nuclear localization in two cells of Cas9-HR8 G3) was seen in all mCherry+ cells regardless of condition, though as quantified, Cas9-HR8 had a significantly higher amount of mCherry+ cells compared to Cas9 or RT. FIG. 11E illustrates an exemplary construct containing the mCherry targeted by two sets of primers and an exemplary agarose gel showing the presence of the intended nucleic acid fragments.

FIG. 12A illustrates an exemplary graph showing the quantifying of normalized Beta-Catenin1:mCherry Knock-in rates in HEK293 cells transfected with B-Cat-G3, Beta-Catenin1:mCherry repair template (RT) and either Cas9-HR8 or Cas9-NT (no tag). Cas9-HR8 shows significant increases in HDR rates (˜2.5×) compared to Cas9. Two replicates were performed per treatment. (P<0.01 two-sided t-test). Demonstrates Cas9-HR8 HDR improvement in an additional cell type. FIG. 12B illustrates an exemplary graph showing the quantifying absolute Beta-Catenin1:mCherry+ cells in HEK293 cells transfected with Beta-Catenin-G1 and Beta-Catenin1:mCherry RT and either Cas9-HR8, Cas9, or Beta-Catenin1:mCherry repair template alone. Cas9-HR8 shows significant increases in mCherry+ cells relative to Cas9-NT or repair template alone (P<0.01 two-sided t-test Cas9-HR8 vs NT or RT). Both Cas9-HR8 and Cas9 show significantly increased numbers of mCherry+ cells compared to repair template (p<0.001 for Cas9-HR8 and NT vs RT). Demonstrates Cas9-HR8 HDR improvement in an additional cell type. FIG. 12C illustrates an exemplary an inverted gray scale image of Beta-Catenin1:mCherry knock-ins in HEK293 cells transfected with B-Cat-G3 and Cas9-HR8 and Beta-Catenin1:mCherry RT. Dark black dots represent mCherry+ HEK293 cells. FIG. 12D illustrates an exemplary inverted grayscale image of Beta-Catenin1:mCherry knock-ins in HEK293 cells transfected with B-Cat-G3 and Cas9-NT and Beta-Catenin1:mCherry RT. Dark black dots represent mCherry+ HEK293 cells. FIG. 12E illustrates an exemplary inverted grayscale image of Beta-Catenin1:mCherry knock-ins in HEK293 cells transfected with Beta-Catenin1:mCherry RT. Dark black dots represent mCherry+ HEK293 cells.

### Example 9. Increased Insertion of Repair Template Encoding Cadherin Induced by Cas Fusion Protein Complex

HEK293T cells were seeded in 24 well plates, grown to ˜70% confluency, then transfected using lipofectamine 3000 with 250 ng of Cas9-HR8 or Cas9, 250 ng of Ecad-G1, and 50 ng of CHD1:mCherry RT. After three days, media was replaced with HBSS (to reduce background for imaging) imaged using an Cytation5 imaging system. Imaging was performed by taking a 6×6 stitched image in the middle of the well using an 10× magnification lens, where both brightfield and RFP images were acquired. HDR rates were quantified via ImageJ, two independent replicates were performed per treatment. FIG. 13A illustrates a Cadherin1:mCherry RT design showing the human genomic region surrounding the last exon (exon 16) of Cadherin1. Two different gRNAs are denoted by black arrows (G1, G2), with arrow direction indicating the targeted strand. A repair template was constructed containing about 750 bp long 5′ and 3′ Homology arms, exon 16 of Cadherin1, silent mutations introduced to prevent gRNA binding to the RT, and a direct fusion of the fluorescent protein mCherry. FIG. 13B illustrates quantification of Cadherin1:mCherry HDR rate in H1299 cells showing the average mCherry+ cells per image section (>10 for all conditions) imaged at day 3 post transfection. Both Cas9-HR8 and Cas9 showed significant increases in positive cell count compared to Cadherin1:mCherry alone. Compared to Cas9, Cas9-HR8 showed significant increases in mCherry+ cells compared to Cas9 for all three gRNAs. When comparing gRNA performance within Cas9-HR8 or Cas9 treated cells, G1 showed the highest HDR efficiency and similar patterns of increases for both Cas9-HR8 and Cas9, again further indicating Cas9-HR8 simply augments HDR efficiency, rather than fundamentally changing the behavior of Cas9. FIG. 13C illustrates graph showing relative fold increase in Cadherin1:mCherry HDR (Cas9-HR8 normalized to Cas9) the normalized fold change of Cas9-HR compared the corresponding guide polynucleotides for Cas9 (e.g. Cas9-HR8 G1/Cas9 G1). All guide polynucleotides showed significant (>1.5 fold) increases in mCherry+ cells relative to Cas9, with G1 showing the highest (˜2.3) fold change. FIG. 13D illustrates representative images of Cadherin1:mCherry+ Cells showing representative images from Bright Field (BF), mCherry (increased visibility of cells) and a merge of the two for Cas9-HR8 and Cas9 G1, and RT only. Cadherin1 is only localized to the membrane, and the expected membrane localization of mCherry (and possible nuclear localization in two cells of Cas9-HR8 G3) was seen in all mCherry+ cells regardless of condition. Additionally, as quantified, Cas9-HR8 had a significantly higher amount of mCherry+ cells compared to Cas9 or RT. FIG. 13E illustrates an exemplary construct containing the mCherry targeted by two sets of primers and an exemplary agarose gel showing the presence of the intended nucleic acid fragments.

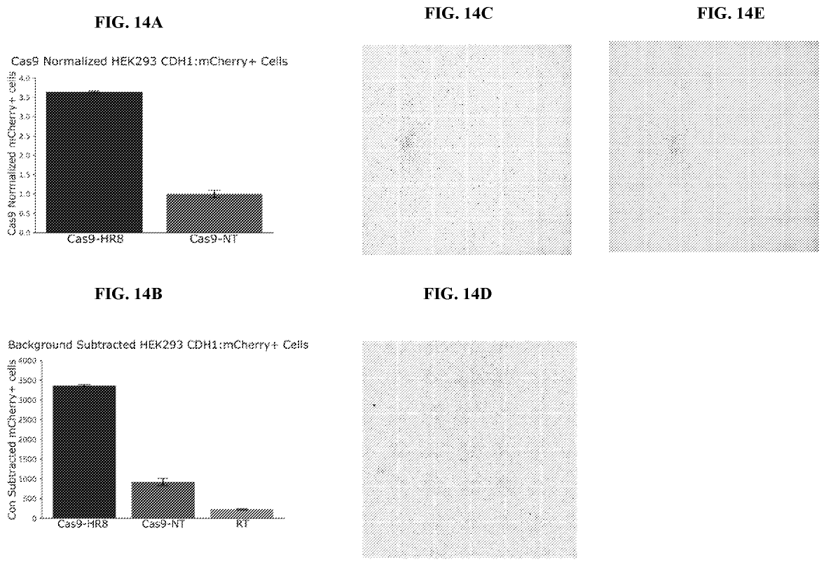

FIG. 14A is an exemplary graph illustrating the quantification of normalized CDH1:mCherry Knock-in rates in HEK293 cells transfected with Ecad-G1, CHD1:mCherry repair template (RT) and either Cas9-HR8 or Cas9-NT (no tag). Cas9-HR8 shows significant increases in HDR rates (˜3.5×) compared to Cas9. Two replicates were performed per treatment. (P<0.0001 two-sided t-test for all) Demonstrates Cas9-HR8 HDR improvement in an additional cell type. FIG. 14B is an exemplary graph showing the quantifying of absolute CDH1:mCherry+ cells in HEK293 cells transfected with Ecad-G1 and CHD1:mCherry RT and either Cas9-HR8, Cas9, or CDH1:mCherry repair template alone. Cas9-HR8 shows significant increases in mCherry+ cells relative to Cas9-NT or repair template alone (P<0.001 two-sided t-test Cas9-HR8 vs NT or RT). Both Cas9-HR8 and Cas9 show significantly increased numbers of mCherry+ cells compared to repair template (p<0.0001 for Cas9-HR8 and NT vs RT). Demonstrates Cas9-HR8 HDR improvement in an additional cell type. FIG. 14C is an exemplary inverted grayscale image of CDH1:mCherry knock-ins in HEK293 cells transfected with Ecad-G1 and Cas9-HR8 and CDH1:mCherry RT. Dark black dots represent mCherry+ HEK293 cells. FIG. 14D is an exemplary inverted grayscale example image of CDH1:mCherry knock-ins in HEK293 cells using Ecad-G1 and Cas9-NT. Dark black dots represent mCherry+ HEK293 cells. FIG. 14E is an exemplary inverted grayscale example image of CDH1:mCherry knock-ins in HEK293 cells using RT only. Dark black dots represent mCherry+ HEK293 cells.

FIG. 15A illustrates whole well imaging of Cas9-HR8 HDR rates of Cadherin1:mCherry genomic integration. Stitched Images from 60 independent sections were taken from cells in a 12 well plate of H1299 cells 4 Days after transfection with Cas9-HR8, Cadherin1:mCherry RT and Ecad-G1 with Brightfield (BF), mCherry, and merged images. Lower images showed enlarged sections (dashed box) as examples of HDR rates. Though high background prevented absolute quantification, Cas9-HR8 showed significant amounts of mCherry+ cells. FIG. 15B illustrates whole well imaging of Cas9 HDR rates of Cadherin1:mCherry genomic integration. Stitched Images from 60 independent sections were taken from cells in a 12 well plate of H1299 cells 4 Days after transfection with Cas9, Cadherin1:mCherry RT and Ecad-G1 with Brightfield (BF), mCherry, and merged images. Lower images showed enlarged sections (dashed box) as examples of HDR rates. Though high background prevented absolute quantification, Cas9 showed low, though detectable amounts of mCherry+ cells. FIG. 15C illustrates whole well imaging of HDR rates of Cadherin1:mCherry genomic integration. Stitched Images from 60 independent sections were taken from cells in a 12 well plate of H1299 cells 4 Days after transfection with Cadherin1:mCherry RT with Brightfield (BF), mCherry, and merged images. Lower images show enlarged sections (dashed box) as examples of HDR rates. Though high background prevented absolute quantification, Cadherin1:mCherry RT alone showed very low rates of mCherry+ cells. FIG. 15D illustrates combined sections of whole well imaging of HDR rates of Cadherin1:mCherry genomic integration. Combined image sections were obtained from cells transfected with either Cas9-HR8 or Cas9, Ecad-G1, Cadherin1:mCherry RT or Cadherin1:mCherry RT alone, shown with Brightfield (BF), mCherry, and merged images. Though high background prevented absolute quantification, Cas9-HR8 shows significantly higher amounts of mCherry+ cells relative to Cas9 or RT alone (Cas9-HR>Cas9>>RT).

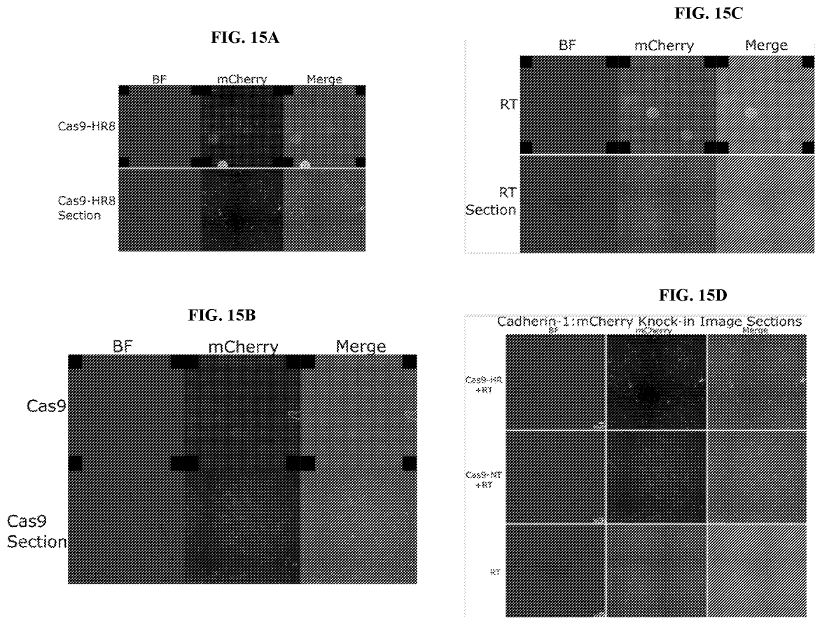

FIG. 16A illustrates HDR rates of fusions of Dna2(1-397)-AP5X-Cas9 and Dna2 (1-397)-Cas9, compared to Cas9-HR8, Cas9 or Cadherin1:mCherry RT alone showing the average mCherry+ cells per image section (>10 for all conditions) imaged at day 3 post transfection. All of Dna2 (1-397)-AP5X-Cas9, Dna2 (1-397)-Cas9, Cas9-HR8 and Cas9 showed significant increases in mCherry+ cell count compared to Cadherin1:mCherry RT alone. Compared to Cas9, Cas9-HR8 showed significant increases in mCherry+ cells, whereas Dna2 (1-397)-AP5X-Cas9 and Dna2(1-397)-Cas9 showed roughly similar levels of mCherry+ cells. FIG. 16B illustrates normalized Cadherin1:mCherry HDR rates of Dna2 (1-397) and Cas9-HR to Cas9 showing the normalized fold change of Dna2 (1-397)-AP5X-Cas9, Dna2 (1-397)-Cas9, and Cas9-HR8 compared to Cas9. Cas9-HR8 had a significantly higher HDR rate than Dna2 (1-397)-AP5X-Cas9, Dna2 (1-397)-Cas9, or Cas9, thereby demonstrating that fusion of the 5′->3′ exonuclease domain of Dna2(1-397) either through a stiff AP5× linker or directly was not sufficient to increase HDR rates.

### Example 10. Increased Insertion of Repair Template in Safe Harbor Site (SHS) Induced by Cas Fusion Protein Complex

H1299 cells were seeded in 24 well plates, grown to ˜70% confluency, then transfected using lipofectamine 3000 with 250 ng of Cas9-HR8 or Cas9, 250 ng of SHS-231-G2, and 50 ng of SHS-231-mNeon RT. After 7 days, cells were tryspinized and transferred to 6 well plates, and grown for an additional 7 days. On Day 14, media was replaced with HBSS (to reduce background for imaging) and imaged using a Cytation5 imaging system. Imaging was performed by taking a 6×6 stitched image in the middle of the well using an 10× magnification lens, where both brightfield and RFP images were acquired. HDR rates were quantified via ImageJ, two independent replicates were performed per treatment. FIG. 17A illustrates that Cas9-HR reduced cellular toxicity in A549 cells compared to Cas9 when targeted to SHS-231. Quantification of cellular viability in A549 cells transfected with Cas9-HR8 or Cas9 targeting either SHS-231 G1, G2, or G3 was measured on Day 2 post transfection via rezarufin/rezazurin metabolic activity assay. Cas9-HR8 showed significant increases in cellular viability relative to Cas9, with G2 and G3 showing greatest increases in cellular viability. FIG. 17B illustrates that Cas9-HR reduced cellular toxicity in A549 cells compared to Cas9 when targeted to Cadherin1. Quantification of cellular viability in A549 cells transfected with Cas9-HR8 or Cas9 targeting either Cadherin1 G1 or G2 was measured on Day 2 post transfection via rezarufin/rezazurin metabolic activity assay. Cas9-HR8 showed significant increases in cellular viability relative to Cas9, with G1 showing the greatest increase in cellular viability, as expected based on pervious correlation of reduction of toxicity and increase in HDR rate. FIG. 17C illustrates that Cas9-HR significantly increases HDR rates for an mNeon expression cassette at a previously identified Safe Harbor Site (SHS)-231. Stitched Images from 78 independent sections were obtained from a 6 well plate of H1299 14 Days after transfection with Cas9-HR8 or Cas9, SHS-231-pCAG-mNeon-bGHPa-RT and SHS-231 G1, with Brightfield (BF), GFP, and merged images. Cas9-HR8 and Cas9 GFP intensities had been increased to aid visualization of GFP+ cells. Cas9-HR showed both significantly more GFP+ cells, as well as significantly more total cells compared to Cas9.

FIG. 18A illustrates SHS-231-pCAG-mNeon-bGHPa RT design showing the human genomic region surrounding SHS-231 (Chr4:58974613-58978632). Three different gRNAs are denoted by black arrows (G1, G2, and G3), with arrow direction indicating the targeted strand. A repair template was constructed containing about 900 bp long 5′ and 3′ homology arms, pCAG (a synthetic strong constitutive promoter), mNeon, and a bGH poly A site (bGHPa). Silent mutations introduced to prevent gRNA binding to the RT are shown in red. FIG. 18B illustrates box plots showing cellular fluorescence levels quantified for mNeon+ cells from either Cas9-HR8 or Cas9 treated cells 14 days transfection. Cas9-HR cells not only showed significantly more mNeon+ cells, but also showed much more uniform and lower expression levels (significantly reduced sizes of quartile ranges and average) compared to Cas9. This is indicative of vastly increased single site, stable integration of SHS-mNeon transgenes relative to Cas9, as cells with single stable integrations would be expected to have significantly lower fluorescence levels than multiple or other improper integration events.

FIG. 19A illustrates in vitro transcription of 5′ Capped and Poly-A tailed Cas9-HR8 mRNA. Cas9-HR8 (with Cas9 as a reference) was in vitro transcribed from a template containing a T7 promoter, strong Kozac initiation sequence, Cas9-HR8 CDS and a about 150 bp poly-A tail. Reactions were run on a 1% TAE gel for about 1 hr, a strong band at ˜2 kb was present in both Cas9-HR8 lanes, indicating transcription of full length Cas9-HR (as expected on a native gel, Cas9 ran at ˜1.8 kb). Additionally, lanes were also run with an extra micro liter of GTP added as GTP could be rate limiting for longer constructs, as shown indicated on the gel. FIG. 19B illustrates that Cas9-HR8 editing produced roughly 10× mNeon+ cells relative to Cas9 at 14 days post-transfection. Quantification of the number of mNeon+ cells in either Cas9-HR8 or Cas9, SHS-231-G1, SHS-231-mNeon RT treated H1299 cells 14 days post-transfection was obtained. FIJI (ImageJ) software was used to identify and quantify mNeon+ cells for both Cas9-HR8 and Cas9, with Cas9-HR8 showing a huge increase (3652 vs 359, >10×) in absolute numbers of mNeon+ cells relative to Cas9.

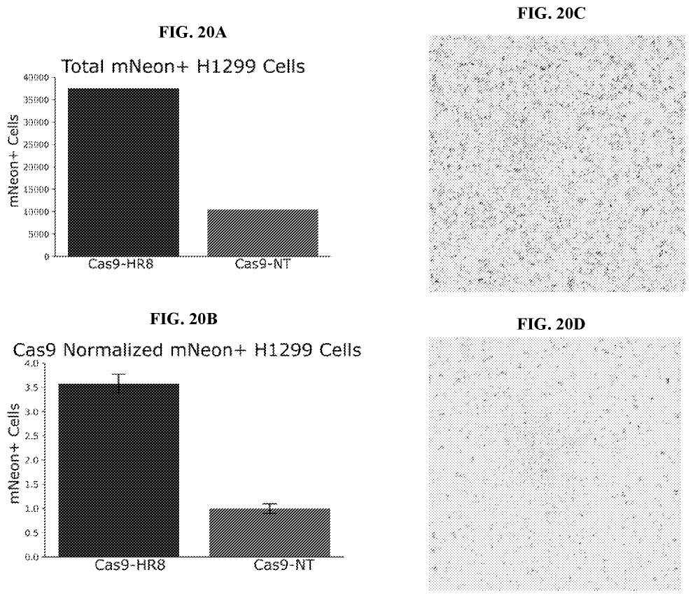

FIG. 20A illustrates an exemplary graph showing total cell counts from two independent experiments transfecting H1299 cells with either Cas9-HR8 or Cas9, SHS-231-G2 and an mNeon transgene. Experiment differs from previous exp due to non-catastrophic death of Cas9 treated cells following trypsinization, proving that increase seen with Cas9-HR8 was not due simply to cell death, but also due to increase of HDR rates. FIG. 20B illustrates an exemplary graph showing normalized cell counts to Cas9 from two independent experiments transfecting H1299 cells with either Cas9-HR8 (red) or Cas9 (NT), SHS-231-G2 and an mNeon transgene (p<0.00001 Cas9-HR8 vs Cas9). Experiment differs from previous experiments due to non-catastrophic death of Cas9 treated cells following trypsinization, proving that increase seen with Cas9-HR8 was not due simply to cell death, but also due to increase of HDR rates. FIG. 20C illustrates an exemplary inverted grayscale image of SHS-231-pCAG-mNeon knock-ins in H1299 cells transfected with SHS-G2 and Cas9-HR8 and SHS-231-pCAG-mNeon RT. Dark black dots represent mNeon+H1299 cells. FIG. 20D illustrates an exemplary inverted grayscale example image of SHS-231-pCAG-mNeon knock-ins in H1299 cells transfected with SHS-G2, Cas9-NT and SHS-231-pCAG-mNeon RT. Dark black dots represent mNeon+H1299 cells.

### Example 11. Increased HDR Rate for Inserting Repair Template in SHS Induced by Cas Fusion Protein Complex

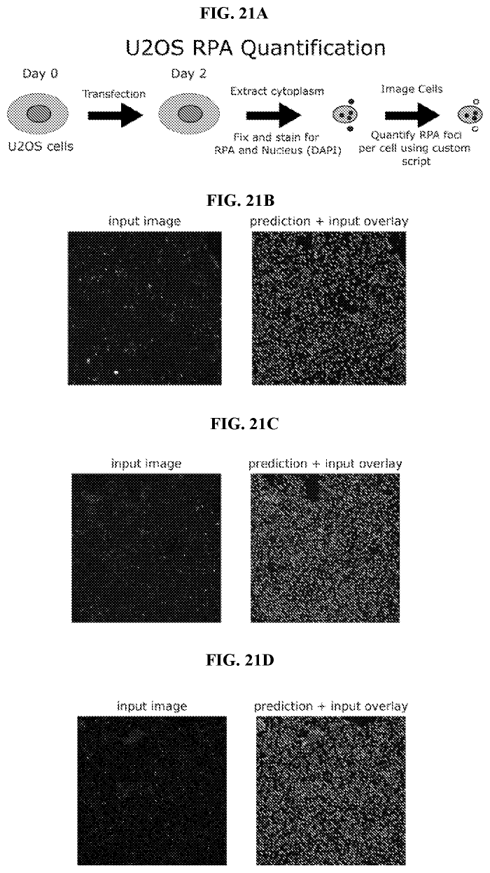

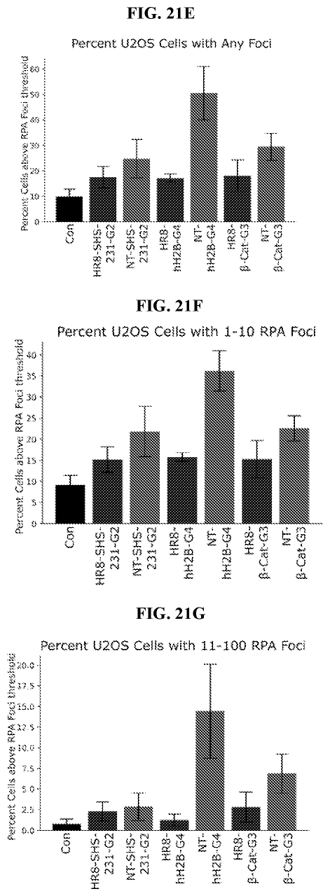

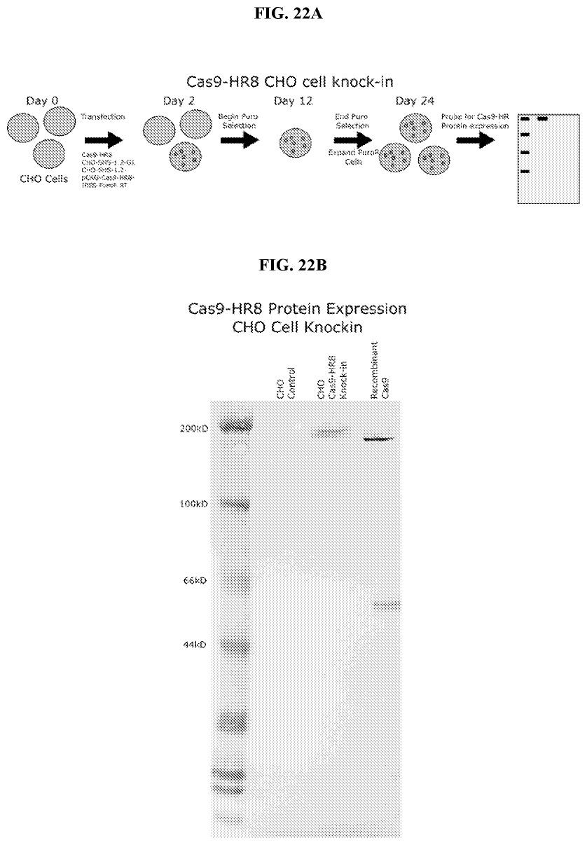

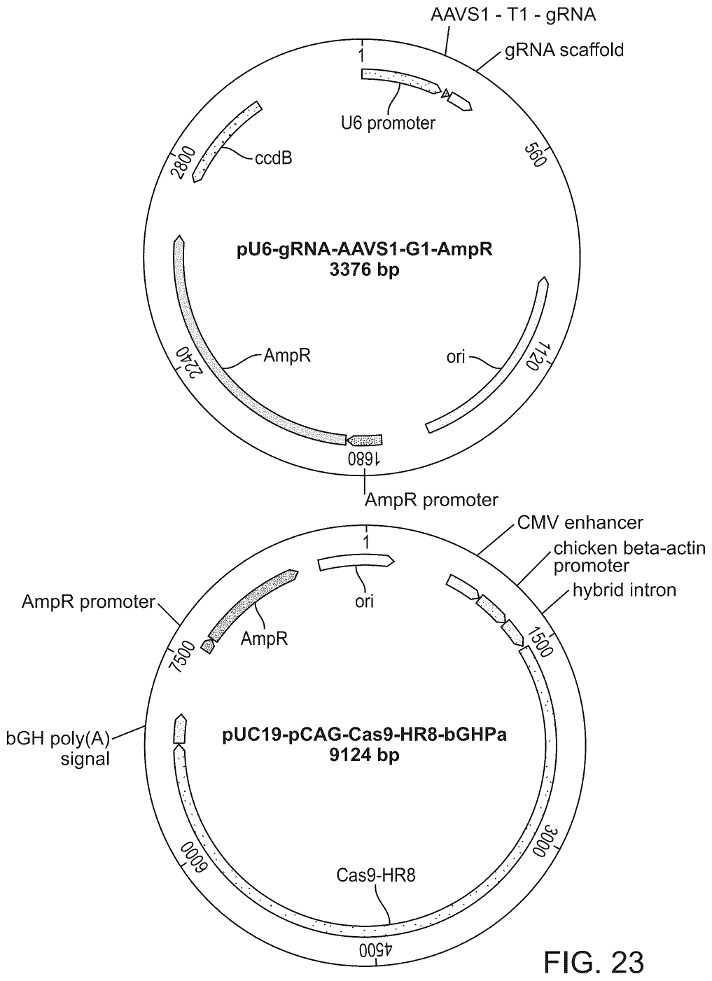

U2OS cells were seeded in glass bottom 96 well plates, grown to ˜70% confluency, and then transfected using lipofectamine 3000 with 250 ng of Cas9-HR8 or Cas9, 250 ng of either SHS-231-G2, Ecad-G1, or B-Catenin-G3. After two days cells, cells were prepared for imaging as in Murkherjee et al 2015. Cytoplasm was extracted via two sequential 10 minute washes on ice consisting of Extraction Buffer 1: 10 mM PIPES, pH 7.0; 100 mM NaCl; 300 mM Sucrose; 3 mM MgCl2; 1 mM EGTA, 0.5% Triton X-100 and Extraction Buffer 2: 10 mM Tris-HCl, pH 7.5; 10 mM NaCl; 3 mM MgCl2, 1% Tween 40, 0.5% sodium deoxycholate. Cells were then fixed using 4% PFA for 20 minutes at RT, then washed three times with PBST, blocked in 5% goat-serum for 30 minutes at RT, then incubated overnight at 4° C. with 1:1000 dilution of mouse anti-RPA (Calbiochem; Catalog no: NA191). Next day cells were washed 5× with PBST with 20 minutes per wash, then incubated overnight at 4° C. with a 1:1000 dilution of anti-mouse Alexa fluor 568 (Thermo Fisher). Cells were washed next day cells were washed 5× with PBST with 20 minutes per wash, then mounted in Vectashield with DAPI (Vector Labs; Catalog no: H-1200), and imaged at 10× using an Leica Stellaris Confocal Microscope. Foci per cell were quantified using a custom script to identify number of RPA foci per cell. FIG. 21A illustrates an exemplary diagram of experiments to quantify RPA Foci in Cas9-HR8 or Cas9 treated U2OS cells. U2OS cells were transfected with Cas9-HR8 or Cas9 targeting either SHS-231, hH2B-G4 or B-Catenin1, or non-treated control cells. After two days cells were fixed, then cytoplasm extracted on ice, then remaining nuclei were stained for RPA. At the end of staining, nuclei were labeled with DAPI and cells were imaged via confocal microscopy. Post-imaging nuclei identification and RPA thresholding allowed quantification of the number of RPA foci per nuclei. FIGS. 21B-D illustrate exemplary confocal images of RPA foci stained U2OS cells transfected with Cas9 (complexed with guide RNA 4 targeting hH2B, FIG. 21B); Cas9-HR8 (complexed with guide RNA 4 targeting hH2B, FIG. 21C); and U2OS control cells (FIG. 21D).

FIG. 21E illustrates an exemplary graph showing percent cells above with any RPA foci in U2OS cells transfected with Cas9-HR8 or Cas9 targeting either SHS-231, hH2B-G4 or B-Catenin1, or non-treated control cells. Both Cas9-HR8 and Cas9 increased the percentage of cells with RPA foci, though Cas9 showed a greater increase relative to Cas9-HR8. FIG. 21F illustrates an exemplary graph showing percent cells with 1-10 RPA foci in U2OS cells transfected with Cas9-HR8 or Cas9 targeting either SHS-231, hH2B-G4 or B-Catenin1, or non-treated control cells. Again, both Cas9-HR8 and Cas9 increased the percentage of cells with RPA foci, though Cas9 showed a greater increase relative to Cas9-HR8, particularly in hH2B and B-Catenin1 treated cells. FIG. 21G illustrates an exemplary graph showing percent cells with 11-100 RPA foci in U2OS cells transfected with Cas9-HR8 or Cas9 targeting either SHS-231, hH2B-G4 or B-Catenin1, or non-treated control cells. Here, Cas9-HR8 showed a significant decrease in cells with 11-100 RPA foci compared to Cas9 targeting both hH2B and Beta-Catenin1, demonstrating that Cas9-HR8 significantly decreased genomic stress (as shown by large amounts of RPA foci) at two independent loci compared to Cas9. Table 1 summarizes exemplary statistical analysis of the measures of FIGS. 21E-21G.

TABLE 1
Statistical analysis of U2OS cells transfected with the Cs9 fusion protein complex

| two sided | | | | | Control | | | | |
|---|---|---|---|---|---|---|---|---|---|
| equal | Control | | Control vs | Control | vs Cas9- | Control | Cas9-HR | Cas9-HR | Cas9-HR |
| variance | vs Cas9- | Control vs | Cas9-HR8 | vs Cas9 | HR8 B- | vs Cas9 | vs Cas9 | vs Cas9 | vs Cas9 |
| t-test | HR8 231 | Cas9 231 | hH2B-G4 | hH2B G4 | Cat-G3 | B-Cat-G3 | 231 | hH2B-G4 | B-Catenin-G3 |
| 1 to 10 | 0.190007 | *0.021895 | 0.169189 | *2.32E−05 | 0.179383 | *0.001589 | 0.295438 | *0.00328 | 0.168149 |
| 11 to 100 | *8.78E−06 | *3.47E−06 | *0.047148 | *7.55E−28 | *3.74E−05 | *2.48E−12 | 0.230703 | *1.41E−26 | *0.000215 |
| all | 0.172289 | *0.026976 | 0.205232 | *2.04E−06 | 0.146969 | *0.000739 | 0.339552 | *0.000328 | 0.113068 |

P < 0.05=*

### Example 12. Stable Knock-In Induced by the Cas Fusion Protein Complex

CHO-Freestyle Cells (Invitrogen) were grown to ˜70% confluency, after which were transfected using the Neon (Thermo) transfection system and 250 ng of Cas9-HR8, 250 ng of either CHO-SHS-1.2-G1, and 50 ng CHO-SHS-1.2-pCAG-Cas9-HR8-IRES-PuroR RT. Cells were allowed to recover for two days, then treated with 10 mg/mL Puromycin for 10 days, after which Puromycin selection was removed. Cells were grown for an additional 12 days, after which cells split and half were frozen, while the other half was treated with RIPA buffer (Santa-Cruz) to extract protein, with non-transfected control CHO cells protein also extracted via RIPA buffer. Lysate was normalized and 7.5 ug total protein was loaded onto a 4-12% Bis-Tris Acrylamide gel (Invitrogen), and transferred to nitrocellulose using BioRad blotting and transfer system (BioRad). Membrane was washed 3× with 1×PBST (BioRad), blocked for 10 minutes using EveryBlot blocking solution (BioRad), and incubated for 1 hr at RT with a 1:1000 dilution of mouse anti-Cas9 (SantaCruz). Membrane was washed 5× for 5 minutes each in PBST, then incubated for an hour with 1:1000 rabbit anti-mouse-HRP secondary antibody for 1 hr at RT. Membrane was then washed 5× for 5 minutes each in PBST, after which a 1× NC/DAB (ThermoFisher) solution was used to visualize bands. FIG. 22A illustrates an exemplary diagram showing CHO cell Cas9-HR8 stable knock-in protocol. Briefly, CHO cells were transfected with Cas9-HR8, CHO-SHS-1.2-G1, and a Cas9-HR8 repair template consisting of: CHO-SHS-1.2-homology arms, pCAG (a strong constitutive promoter), Cas9-HR8, an IRES sequence, a Puromycin resistance gene, and a BGH poly adenylation signal, totaling over 8 kb. After recovering from electroporation, cells were treated with Puromycin for 10 days, after which they were grown an additional 12 days without puromycin. A portion of cells were lysed while others were frozen, and either control or Cas9-HR8 knock-in CHO cells were probed for expression via anti-Cas9 western blot. FIG. 22B illustrates Cas9-HR8 CHO cells exhibiting strong staining at ˜200 kD, which is the predicted size for Cas9-HR8 thereby demonstrating that the Cas9-HR8 CHO cell line has stably integrated Cas9-HR8 and expression remaining stable for long-term growth. Purified recombinant Cas9 was included as a sizing comparison.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A method for introducing an edit into a genomic locus of a plurality of cells, the method comprising:
contacting the plurality of the cells with:
a) a Cas fusion protein complex comprising a Cas fusion protein complexed with a guide polynucleotide configured to bind to the genomic locus of a cell of the plurality of cells; and
b) a repair template comprising at least 5000 base pairs (bp) in length,
thereby introducing the edit into the genomic locus of the plurality of cells with the repair template, wherein the edit comprises a homology directed repair (HDR).

2-4. (canceled)

5. The method of claim 1, wherein at least 50% of the plurality of cells remain viable after introducing the edit into the genomic locus of the plurality of cells with the repair template.

6-9. (canceled)

10. The method of claim 1, wherein the Cas fusion protein comprises a Cas nuclease fused to an exonuclease or a fragment thereof.

11. The method of claim 1, wherein the Cas fusion protein comprises a Cas9 nuclease or a Cas12 nuclease.

12. The method of claim 11, wherein the Cas9 nuclease comprises a polypeptide sequence that is at least 75% identical to any one of SEQ ID NOs: 7-23.

13-18. (canceled)

19. The method of claim 11, wherein the Cas12 nuclease comprises a polypeptide sequence that is at least 75% identical to any one of SEQ ID NOs: 55-57.

20-24. (canceled)

25. The method of claim 10, wherein the Cas fusion protein comprises the Cas nuclease fused to a Human Exo1 (hExo1) or fragment thereof.

26. (canceled)

27. The method of claim 25, wherein the hExo1 comprises a polypeptide sequence that is at least 75% identical to SEQ ID NO: 1 or SEQ ID NO: 2.

28-39. (canceled)

40. The method of claim 1, wherein the Cas9 fusion protein comprises the Cas fused to a DNA replication ATP-dependent helicase/nuclease (DNA2) or a fragment thereof.

41. (canceled)

42. The method of claim 40, wherein the DNA2 comprises a polypeptide sequence that is at least 75% identical to SEQ ID NO: 4 or SEQ ID NO: 5.

43-54. (canceled)

55. The method of claim 1, wherein the repair template comprises a mutated protospacer adjacent motif (PAM) sequence located at the immediate 3′ end of a cleavage site, wherein said mutated PAM sequence comprises 5′-NCG-3′ or 5′-NGC-3′.

56. The method of claim 55, wherein the mutated PAM sequence is not cleaved by the Cas fusion protein.

57-67. (canceled)

68. The method of claim 1, wherein the genomic locus encodes a gene associated with cancer selected from the group consisting of cadherin and catenin.

69. The method of claim 1, wherein the genomic locus encodes a gene selected from the group consisting of an oncogene or a tumor suppressor gene.

70-75. (canceled)

76. The method of claim 1, wherein the genomic locus comprises a safe harbor site (SHS).

77-78. (canceled)

79. The method of claim 1, wherein the Cas fusion protein increases a rate of HDR in the plurality of the cells compared to a rate of HDR induced by a second Cas protein in a plurality of cells lacking the Cas fusion protein.

80. The method of claim 79, wherein the Cas fusion protein increases the rate of HDR in the plurality of the cells by at least 10% or more compared to the rate of HDR induced by a second Cas protein in the plurality of cells lacking the Cas fusion protein.

81-84. (canceled)

85. The method of claim 1, wherein the Cas fusion protein decreases an endogenous p53 activity in the plurality of the cells compared to an endogenous p53 activity induced by a second Cas protein in a plurality of cells lacking the Cas fusion protein.

86-90. (canceled)

91. The method of claim 79, wherein the second Cas protein is a wild type Cas9 nuclease.

92-96. (canceled)


SEQUENCE LISTING (236 sequences)

SEQ ID NO: 1 - protein, 846 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 2 - protein, 392 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKE

SEQ ID NO: 3 - protein, 454 aa, Homo sapiens
NPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 4 - protein, 1060 aa, Homo sapiens
MEQLNELELLMEKSFWEEAELPAELFQKKVVASFPRTVLSTGMDNRYLVLAVNTVQNKEGNCEKRLVITASQSLENKELCILRNDWCSVPVEPGDIIHLEGDCTSDTWIIDKDFGYLILYPDMLISGTSIASSIRCMRRAVLSETFRSSDPATRQMLIGTVLHEVFQKAINNSFAPEKLQELAFQTIQEIRHLKEMYRLNLSQDEIKQEVEDYLPSFCKWAGDFMHKNTSTDFPQMQLSLPSDNSKDNSTCNIEVVKPMDIEESIWSPRFGLKGKIDVTVGVKIHRGYKTKYKIMPLELKTGKESNSIEHRSQVVLYTLLSQERRADPEAGLLLYLKTGQMYPVPANHLDKRELLKLRNQMAFSLFHRISKSATRQKTQLASLPQIIEEEKTCKYCSQIGNCALYSRAVEQQMDCSSVPIVMLPKIEEETQHLKQTHLEYFSLWCLMLTLESQSKDNKKNHQNIWLMPASEMEKSGSCIGNLIRMEHVKIVCDGQYLHNFQCKHGAIPVTNLMAGDRVIVSGEERSLFALSRGYVKEINMTTVTCLLDRNLSVLPESTLFRLDQEEKNCDIDTPLGNLSKLMENTFVSKKLRDLIIDFREPQFISYLSSVLPHDAKDTVACILKGLNKPQRQAMKKVLLSKDYTLIVGMPGTGKTTTICTLVRILYACGFSVLLTSYTHSAVDNILLKLAKFKIGFLRLGQIQKVHPAIQQFTEQEICRSKSIKSLALLEELYNSQLIVATTCMGINHPIFSRKIFDFCIVDEASQISQPICLGPLFFSRRFVLVGDHQQLPPLVLNREARALGMSESLFKRLEQNKSAVVQLTVQYRMNSKIMSLSNKLTYEGKLECGSDKVANAVINLRHFKDVKLELEFYADYSDNPWLMGVFEPNNPVCFLNTDKVPAPEQVEKGGVSNVTEAKLIVFLTSIFVKAGCSPSDIGIIAPYRQQLKIINDLLARSIGMVEVNTVDKYQGRDKSIVLVSFVRSNKDGTVGELLKDWRRLNVAITRAKHKLILLGCVPSLNCYPPLEKLLNHLNSEKLIIDLPSREHESLCHILGDFQRE

SEQ ID NO: 5 - protein, 397 aa, Homo sapiens
MEQLNELELLMEKSFWEEAELPAELFQKKVVASFPRTVLSTGMDNRYLVLAVNTVQNKEGNCEKRLVITASQSLENKELCILRNDWCSVPVEPGDIIHLEGDCTSDTWIIDKDFGYLILYPDMLISGTSIASSIRCMRRAVLSETFRSSDPATRQMLIGTVLHEVFQKAINNSFAPEKLQELAFQTIQEIRHLKEMYRLNLSQDEIKQEVEDYLPSFCKWAGDFMHKNTSTDFPQMQLSLPSDNSKDNSTCNIEVVKPMDIEESIWSPRFGLKGKIDVTVGVKIHRGYKTKYKIMPLELKTGKESNSIEHRSQVVLYTLLSQERRADPEAGLLLYLKTGQMYPVPANHLDKRELLKLRNQMAFSLFHRISKSATRQKTQLASLPQIIEEEKTCKYCS

SEQ ID NO: 6 - protein, 663 aa, Homo sapiens
QIGNCALYSRAVEQQMDCSSVPIVMLPKIEEETQHLKQTHLEYFSLWCLMLTLESQSKDNKKNHQNIWLMPASEMEKSGSCIGNLIRMEHVKIVCDGQYLHNFQCKHGAIPVTNLMAGDRVIVSGEERSLFALSRGYVKEINMTTVTCLLDRNLSVLPESTLFRLDQEEKNCDIDTPLGNLSKLMENTFVSKKLRDLIIDFREPQFISYLSSVLPHDAKDTVACILKGLNKPQRQAMKKVLLSKDYTLIVGMPGTGKTTTICTLVRILYACGFSVLLTSYTHSAVDNILLKLAKFKIGFLRLGQIQKVHPAIQQFTEQEICRSKSIKSLALLEELYNSQLIVATTCMGINHPIFSRKIFDFCIVDEASQISQPICLGPLFFSRRFVLVGDHQQLPPLVLNREARALGMSESLFKRLEQNKSAVVQLTVQYRMNSKIMSLSNKLTYEGKLECGSDKVANAVINLRHFKDVKLELEFYADYSDNPWLMGVFEPNNPVCFLNTDKVPAPEQVEKGGVSNVTEAKLIVFLTSIFVKAGCSPSDIGIIAPYRQQLKIINDLLARSIGMVEVNTVDKYQGRDKSIVLVSFVRSNKDGTVGELLKDWRRLNVAITRAKHKLILLGCVPSLNCYPPLEKLLNHLNSEKLIIDLPSREHESLCHILGDFQRE

SEQ ID NO: 7 - protein, 1367 aa, Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 8 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 9 - protein, 1629 aa, Francisella tularensis
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 10 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 11 - protein, 1388 aa, Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 12 - protein, 1101 aa, Actinomyces naeslundii
MWYASLMSAHHLRVGIDVGTHSVGLATLRVDDHGTPIELLSALSHIHDSGVGKEGKKDHDTRKKLSGIARRARRLLHHRRTQLQQLDEVLRDLGFPIPTPGEFLDLNEQTDPYRVWRVRARLVEEKLPEELRGPAISMAVRHIARHRGWRNPYSKVESLLSPAEESPFMKALRERILATTGEVLDDGITPGQAMAQVALTHNISMRGPEGILGKLHQSDNANEIRKICARQGVSPDVCKQLLRAVFKADSPRGSAVSRVAPDPLPGQGSFRRAPKCDPEFQRFRIISIVANLRISETKGENRPLTADERRHVVTFLTEDSQADLTWVDVAEKLGVHRRDLRGTAVHTDDGERSAARPPIDATDRIMRQTKISSLKTWWEEADSEQRGAMIRYLYEDPTDSECAEIIAELPEEDQAKLDSLHLPAGRAAYSRESLTALSDHMLATTDDLHEARKRLFGVDDSWAPPAEAINAPVGNPSVDRTLKIVGRYLSAVESMWGTPEVIHVEHVRDGFTSERMADERDKANRRRYNDNQEAMKKIQRDYGKEGYISRGDIVRLDALELQGCACLYCGTTIGYHTCQLDHIVPQAGPGSNNRRGNLVAVCERCNRSKSNTPFAVWAQKCGIPHVGVKEAIGRVRGWRKQTPNTSSEDLTRLKKEVIARLRRTQEDPEIDERSMESVAWMANELHHRIAAAYPETTVMVYRGSITAAARKAAGIDSRINLIGEKGRKDRIDRRHHAVDASVVALMEASVAKTLAERSSLRGEQRLTGKEQTWKQYTGSTVGAREHFEMWRGHMLHLTELFNERLAEDKVYVTQNIRLRLSDGNAHTVNPSKLVSHRLGDGLTVQQIDRACTPALWCALTREKDFDEKNGLPAREDRAIRVHGHEIKSSDYIQVFSKRKKTDSDRDETPFGAIAVRGGFVEIGPSIHHARIYRVEGKKPVYAMLRVFTHDLLSQRHGDLFSAVIPPQSISMRCAEPKLRKAITTGNATYLGWVVVGDELEINVDSFTKYAIGRFLEDFPNTTRWRICGYDTNSKLTLKPIVLAAEGLENPSSAVNEIVELKGWRVAINVLTKVHPTVVRRDALGRPRYSSRSNLPTSWTIE

SEQ ID NO: 13 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 14 - protein, 1334 aa, Listeria innocua
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 15 - protein, 1056 aa, Pasteurella multocida
MQTTNLSYILGLDLGIASVGWAVVEINENEDPIGLIDVGVRIFERAEVPKTGESLALSRRLARSTRRLIRRRAHRLLLAKRFLKREGILSTIDLEKGLPNQAWELRVAGLERRLSAIEWGAVLLHLIKHRGYLSKRKNESQTNNKELGALLSGVAQNHQLLQSDDYRTPAELALKKFAKEEGHIRNQRGAYTHTFNRLDLLAELNLLFAQQHQFGNPHCKEHIQQYMTELLMWQKPALSGEAILKMLGKCTHEKNEFKAAKHTYSAERFVWLTKLNNLRILEDGAERALNEEERQLLINHPYEKSKLTYAQVRKLLGLSEQAIFKHLRYSKENAESATFMELKAWHAIRKALENQGLKDTWQDLAKKPDLLDEIGTAFSLYKTDEDIQQYLTNKVPNSVINALLVSLNFDKFIELSLKSLRKILPLMEQGKRYDQACREIYGHHYGEANQKTSQLLPAIPAQEIRNPVVLRTLSQARKVINAIIRQYGSPARVHIETGRELGKSFKERREIQKQQEDNRTKRESAVQKFKELFSDFSSEPKSKDILKFRLYEQQHGKCLYSGKEINIHRLNEKGYVEIDHALPFSRTWDDSFNNKVLVLASENQNKGNQTPYEWLQGKINSERWKNFVALVLGSQCSAAKKQRLLTQVIDDNKFIDRNLNDTRYIARFLSNYIQENLLLVGKNKKNVFTPNGQITALLRSRWGLIKARENNNRHHALDAIVVACATPSMQQKITRFIRFKEVHPYKIENRYEMVDQESGEIISPHFPEPWAYFRQEVNIRVFDNHPDTVLKEMLPDRPQANHQFVQPLFVSRAPTRKMSGQGHMETIKSAKRLAEGISVLRIPLTQLKPNLLENMVNKEREPALYAGLKARLAEFNQDPAKAFATPFYKQGGQQVKAIRVEQVQKSGVLVRENNGVADNASIVRTDVFIKNNKFFLVPIYTWQVAKGILPNKAIVAHKNEDEWEEMDEGAKFKFSLFPNDLVELKTKKEYFFGYYIGLDRATGNISLKEHDGEISKGKDGVYRVGVKLALSFEKYQVDELGKNRQICRPQQRQPVR

SEQ ID NO: 16 - protein, 1084 aa, Corynebacterium diphtheriae
MKYHVGIDVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 17 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 18 - protein, 1073 aa, unidentified
  source 1..1073: note = Rhodobacteraceae bacterium sequence
MRLGLDIGTNSIGWWLCETDRADARVRINGVLAGGVRIFSDGRDPKSRASLAVDRRAARAMRRRRDRYLRRRATLMKVLANAGLMPSTPEEAKALELLDPYELRATGLDQILPLTHLGRALFHINQRRGFKSNRKTDWGDNESGKIKDATARLDLAILANGARTYGEFLHKRRQRAVDPRHVPTVRTRLSIANRDGPDGKEEAGYDFYPDRKHLEEEFRKLWAAQANFHPELTEDLHDLIFEKIFYQRPLKEPKVGLCLFTSEERLPKAHPLTQARVLYETVNQLRVIADGRETRRLTLEERDQIIYVLDNKKPTVSLKSMAMKLPALARTLKLRDGERFTLETGVRDAIACDPVRSSLSHPDRFGPRWSTLDATAQWEVVSRVRKVQSEAEHAALVDWLMQAYSIDRNHAEATANAPLPEGFGRLGQTATTSILERLKADVVTYAEAVAACGWHHSDQRTGECLDRLPYYGEVLDRHVIPGTYDANDDEVTRYGRITNPTVHIGLNQLRRLVNRIIETYGKPDQIVLELARELKQSEQQKRDAIKRIRDTTEAAKKRSEKLEELGIEDNGRNRMLLRLWEDLNPEDAMRRFCPYTGERISATMIFDGSCDVDHILPYSRTLDDSFANRTLCLKEANREKRNQTPWKAWGDAPKWDTIEAKLKNLPENKRWRFAPDAMERFEGEKDFLDRALVDTQYLARISRTYMDTLFSEGGHVWVVPGRLTEMLRRHWGLNSLLSDKDRGAVKAKNRTDHRHHAIDAAVVAATDRSLLNRISRAAGQGEAAGQSAELIARDTPPPWEGFRDDLRVQLDKIIVSHRADHGRIDREGRKQGRDSTAGQLHNDTAYGVVDAMTVVSRTPLLSLKPSDIAVTPKGKNIRDPQLQKALEIATRGKEGKAFEAALRQFAEKAGAYQGLRRVRLIETLQESARVEIGTRSEGGPLKAYKGDSNHCYELWRLPDGKVKPQVVTTYEAHAGIEKRPHPAAKRLLRTFKRDMVALERNGETVICYVQKFNQAGILFLASHLESNADARDRDPNDSFTLFRMSPGPMHKAGIRRVSVDEIGRLRDGGAETH

SEQ ID NO: 19 - protein, 965 aa, Campylobacter coli
MKIIGFNLGIANIGWALRENDEIIDCGVRVFDIPENPKNGNSLALERRENKARMKIVKRKKARMLATKTFLKKEFNVDLSKLFLIGSTQSIYELRTKALSSLISKEELSAIILHIAKHRGYDDSALKNENGTIIEALNKNKEAMLKFKSVGEYFYKNFVQNKEVKKIRNTTEDYSNSVPRSLLKQELDLILDKQKELGLIKNADFKAKLFEIIFFKRPLKDFSNKIGNCIFFENEKRAAKNTISACEFVALGKVVNLLKSIEKDIGIVYEKDSINEIMSIILDKTSISYKKIRDILNLPQDINFKGLDYSKNNVENSKLVDLKKLNEFKKALGDGFTNLDKDILDSIATDITLTKDTATLKEKLKNYNVLNAEQIEKLSELVFNDHINLSLKALKQIIPLMYEGKRYDEACELCNFTIAKNQEKNEYLPLFEKTRFAKDISSPVVIRAICEFRKLLNDIIRRYGSVHKIHLELTRDFGISFNDRKKIIKEIEQNEQSRIKALETIKELKLEETSKNIQIVRLFEDQKGICPYSGLKMDLKCLDELVIDYIRPYNRSLDDSYSNKVLTFKKLNDLKQGKTPFEAFGEDEKLWAEINERIKEYNGKKRFKIFDKFFKDKKPFDFTEQTLQDTRWLTKLVASYLNEYLSFLPISEDENTALGYGEKGSKQHVILSSGMITQMLRNFWYLGFKNHKDYKNNAMDAIIVAFTTNSIIFTFNNFKKELDLAKAEFYANKISESDYLLKRKFLPPFSGFKEQALEKVKNIFVSHSLKIKNKGTLHELTPLKIKELKNTYGDLDLAVKLGKIRKYNDKYYANAKGSLVRTDLFVDKENKFHAVSIYKADFSTKKLPNKTPATTSNGETKEGIEMNENYNFCMSLYKNTPIGVKIKGMKESIICYYHGFNTSGSKITYKKHDNNYHNLSEDEMVVFRKNDKESIVVGKILEIKKYSISPSGELSLIENEKRKWF

SEQ ID NO: 20 - protein, 1466 aa, unidentified
  source 1..1466: note = Ignavibacteria bacterium sequence
MKNILGLDLGTNSIGWALIDKENNKIIDMGSRIIPMSQDILGEFGKGNSISQTAERTNYRSIRRLRERYLLRRERLHRVLNILEFLPKHYSDQIDFETRLGKFKEDTEPKIAYKSTIDETNSKSRFDFIFKKSFAEMLEDFHQYQPELFANDNKIPYDWTIYFLRKKALTKKIEKEELAWILLNFNQKRGYYQLREELEEDTNKKEYVVSLKVIKIVKGEEDKKNKNRNWYSISLENGWVYNATFSTEPQWLMTEKEFLVTEELDENGQVKIVKDKKSDKEGKEKRRIIPLPSFDEINLMSKSEPDRIYKKIKAKTETAISNSGKTVGEYIYENLLQNPSQKIRGKLIRTIERKFYKEELKQILQKQKEFHPELQNDDLYNDCVRELYKNNEGHQFLLSKRDFIHLLLDDIIFYQRPLKSQKSLISNCTFEFKKYNVGNEEKIKYLKAIPKSHPLYQEFRFWQWIYNLRVYRKDDDQDVTNDYLNDPEKYADLFEFLSNRKEIDQKALLKYFKLKESTHRWNFVEDKKYPCFETRTLISTRLEKVKDLPPNFLTDQTELQLWHIIYSVTDKIEFEKALSTFAKRNKLDVTTFVENFKKFPPFKSEYGSYSGKALKKLLPLMRSGRYWKWDDIDEKTKTRIDKIITGEFDEDIKNKVREKSINLTTENHFQGLQVWLASYIVYDRHAEAATINKWDTIEHLENYIKEFKQHSLRNPIVEQVTLEALRVIKDIWKQFGKSAENFFDEIHIELGREMKNTADERKRLTSQINDNENTNVRIKALLAELKNDSNIENVRPFSPIQQELLKIYEDGVLNSEIEIPDDISKISKTAQPSSSELQRYKLWLEQKYRSPYTGQVIPLAKLFTTDYEIEHIIPQSRYFDDSFNNKVICEAAVNKLKDNQTGLEFIKNHHGEIVQTVFDNKVKIFEENDYRDFVKTHYIKNRSKRNKLLMEEIPDKMIERQINDTRYITKFISALLSNIVRAENNDEGLNSKNLIQVNGKITSLLRQDWGINDIWNDLILPRFLRMNQITNSDAFTRYNDKYQKYLPTVPLELSKNYQSKRIDHRHHALDALIIACATRDHVNLLNNKYAKSKERYDLNRKLRLFEKVVYTHPKTGEKIEREIPKNFIKPWDTFTVDTKNFLDTIVVSFKQNLRIINKATNQYQKWVKLNGRNVKKEVKQSGINWAIRKPLHKETVAGKVELKRIKVPKGKILTATRKNLDTSFDIKTIESITDTGIQKILKNYLSAKGNDPTIAFSPEGIEEMNKNITRYNNGKPHRPIYKARIFELGSKFILGLTGNKKAKYVEAAKGTNLFYAIYVDENNKRSFETIPLNIVIERQKQGLSSVPENDDKGNKLLFYLSPNDLVYVPDEDEIINESYLDVSNLSNEQKKRLYNVNDFSSTCYFTPNRIAKAIAPKEVDLNYDNNKKKLFGSYDTKTASVNGIQIKDICIKLKADRLGNISKANR

SEQ ID NO: 21 - protein, 1319 aa, Fructobacillus sp.
MGYNIGLDIGTGSVGWAALTDEGKLARAKGKNLIGVRLFDSAQSAAQRRSYRTTRRRLSRRKWRLRLLENIFSDEMGMIDENFFARLKYSYVHPKDEVNNAHYYGGYLFPTQQETHDFHEKFQTIYHLRLKLMIEDCKFDLREIYLAMHHIVKYRGHFLNSQSKMTIGDSYNPRDFQQAIQNYAEAKGLIWSLNDAQEMTDVLVGQAGFGLSKKAKAERLLSAFSFDTKEDKKAIQAILAGIVGNTTDFTKIFNRERSGDELKKWKLKLDSEAFDEQSQAIVDELDDDEMELFNAIRQAFDGFTLMDLLGDQTSISAAMVKRYQQHHDDLKMVKEIAKKQGLSHQDFSKIYTAFLKDDTDKGMKALLDKADLADDVLVEIQQRIESHDFLPKQRTKANSVIPYQLHLAELEKIIENQGKYYPFLLDTFTNKAGETINKLVELVKFRVPYYVGPMVTAADVEKAGGDATNHWVKRNEGYEKSPVTPWNFDQVFNRDQAAQDFIDRLTGTDTYLIGEPTLLKNSLKYQLFTVLNELNNVKINGHKIDEKTKHVLIQDLFKSKKTVSEKAIKDYYLSQGMGEIQIVGLADKTKFNSNLSSYIDLSKTFDAEFMENPANQELLENIIQIQTVFEDVKIAERELQKLALPDEQVQQLAKTHYTGWGNLSDKLLSTPIIQEGSQKVSILNKLQTTSKNFMSIITDNKFGVQQWIQEQNTAETADSIQDRIDELTTAPANKRGIKQAFNVLFDIQKAMGEEPNRVYLEFAKETQNSVRTNSRYNRLKDLYKSKTLSDDVKALKEELESQKSSLQSERIGDRLYLYFLQQGKDMYTGQPINIDKLSTDYDIDHIIPQAYTKDDSIDNRVLVSRPENARKSDSATYTTEVQQSAGGLWKSLKNAGFISQKKYDRLTKGGDYSKGQKTGFIARQLVETRQIIKNVASLIESEFSQTKAVAIRSEITADMRRLVAIKKHREINSFHHAFDALLITAAGQYMQARYPDRDGANVYNEFDYYTNTYLKELRQSSSSSQVRRLKPFGFVVGTMAKGNENWSEDDTQYLRHVMNFKNILTTRRNDKDNGALNKETIYAVDPKAKLIGTNKKRQDVSLYGGYIYPYSAYMTLVRANGKNLLVKVTISAAEKIKSGQIELSEYVQQRPEVKKFEKILINKLAIGQLVNNDGNLIYLTSYEFYHNAKQLWLPTEEADLISQLNKDSSDEDLIKGFDILTSPAILKRFPFYELDLKKLVNIRDKFIAVENKFDILMVILKALQLDAAQQKPVKMIDKKSADWKDYRQRGGIKLSDTSEIIYQSTTGIFEKRVKISNLL

SEQ ID NO: 22 - protein, 1274 aa, Pedobacter glucosidilyticus
MTKHILGLDLGTNSIGWAIIQVDNNNNVPIQIIAMGSRIIPLDSNDRDQFQKGQAISKNKDRTTARTQRKGYDRKQLKKSDDFKYSLKKILEKLDIFPTEELMKLPTLDLWKLRSDAVSNIEDITPKQLGRILYMLNQKRGYKSARSEANADKKDTDYVAEVKGRYTQLKDKGQTLGQYFYKELSDANQNNTYYRVKEKVYPREAYIEEFDAIINVQKSKHSFLTDEVIHSLRNEIIYYQRKLKSQKGLVSICEFEGFETTYFDKKTQQDKTIFTGPKVAPRTSPLFQFCKIWEVVNNISLKTKNPEGSKYKWSDRIPTIEEKQTIANYLQENENLSFIELLKILQLKKEQVYANKQILKGIQGNTTFSAIHKIIGNSEHLKFDIETIPSKHFAVLVDKKTGEILDERDSLELNSALEQEPFYQLWHTIYSIKDLDECKKALIKRFNFEEEIAEKLSKIDFNKQAFGNKSNKAMRKMLPYLMLGYNQSEAESFAGYNRRLTKEEKSKNVSDEPLQLLAKNSLRQPVVEKILNQMINVVNAIIEKYGKPEEIRVELARELKQSKDEREDADKQNGFNKKLNELVATKLTELGLPTTKHYIQKYKFIFPAKDKNWKEAQVANQCIYCGDTFNLTEALSGDNFDVDHIVPKALLFDDSQANKVLVHRSCNSTKTNNTAYDYITKKGSQALNDYVARVDDWFKRGIISYGKMQRLKVSFEEYQERKKIGKETEADKRIWENFIDRQLRETAYIAKKAKEILEKVCHNVTSTEGNVTAKLRQLWGWDNVLMNLQLPKYKELEKKTKQTFTQLKEWTSDHGNRKHQKEEIINWTKRDDHRHHAIDALVIACTQQGFIQRINTLSSSDVKDEMKKELEEDKTVYNERLTLLENYLLEKKPFSTEEIEKEADKILVSFKAGKKVATLSKYKATGINEIKGVLVPRGPLHEQSVYGKIKVIEKDKPLKYLFENSDKIVNPLIKHLVKTRLLENENNAQAALVTLKNKPILLNNKQTEILEKASCYNEATVLKYKLQSLKASQIDDIVDEKIKFLIKERLSKFGNKEKEAFKDILWFNEKKQIPITSIRLFARPDANNLQVIKKHEKGKNIGFVLSGNNHHIAIYEDKNNKLIQHICDFWHAVERKRNNIPVLIEDTSTIWNHLINEDFSESFLNKLPNDSLKLKFSLQQNEMFILGLPKEQSEEAIKSNNKSLLSKHLYLVWSITDGDYFFRHHLETKNTELKKIDGSKESKRYLRLSTKSLVDLNPIKVRLNHLGEITKIGE

SEQ ID NO: 23 - protein, 1082 aa, Geobacillus thermodenitrificans
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 24 - DNA, 4158 bp, synthetic construct
  source 1..4158: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaaacaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcaataccgcagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaataa

SEQ ID NO: 25 - DNA, 4158 bp, synthetic construct
  source 1..4158: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaataa

SEQ ID NO: 26 - DNA, 4158 bp, synthetic construct; identical to SEQ ID NO: 24
  source 1..4158: mol_type = other DNA

SEQ ID NO: 27 - DNA, 4167 bp, synthetic construct
  source 1..4167: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagcgcacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaaacaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgacccgtctgggagcccctcgcgccttcaagtactttgacaccaccatcgacccgaagcaataccgcagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaataagaattctaa

SEQ ID NO: 28 - DNA, 4167 bp, synthetic construct
  source 1..4167: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccgccttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcgcgctgagccggaagctgatcaacggcatccgggacaagcagtccggcaagacaatcctggatttcctgaagtccgacggcttcgccaacagaaacttcatggcgctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccgggcgatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaataagaattctaa

SEQ ID NO: 29 - DNA, 4167 bp, synthetic construct
  source 1..4167: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctggcggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctgcgctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaaggcgcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaataagaattctaa

SEQ ID NO: 30 - DNA, 3972 bp, synthetic construct
  source 1..3972: mol_type = other DNA
atgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatagaaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctggccctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggccagaggctgggacgtgaatagagagaagaacaatggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgatcggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaacaaaaggccggcggccacgaaaaaggccggccaggcaaaaaagaaaaagtaa

SEQ ID NO: 31 - DNA, 1716 bp, synthetic construct
  source 1..1716: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgag

SEQ ID NO: 32 - DNA, 2448 bp, synthetic construct
  source 1..2448: mol_type = other DNA
tgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaataagaattctaa

SEQ ID NO: 33 - DNA, 3273 bp, synthetic construct
  source 1..3273: mol_type = other DNA
atggctgccttcaaacctaattcaatcaactacatcctcggcctcgatatcggcatcgcatccgtcggctgggcgatggtagaaattgacgaagaagaaaaccccatccgcctgattgatttgggcgtgcgcgtatttgagcgtgccgaagtaccgaaaacaggcgactcccttgccatggcaaggcgtttggcgcgcagtgttcgccgcctgacccgccgtcgcgcccaccgcctgcttcggacccgccgcctattgaaacgcgaaggcgtattacaagccgccaattttgacgaaaacggcttgattaaatccttaccgaatacaccatggcaacttcgcgcagccgcattagaccgcaaactgacgcctttagagtggtcggcagtcttgttgcatttaatcaaacatcgcggctatttatcgcaacggaaaaacgagggcgaaactgccgataaggagcttggcgctttgcttaaaggcgtagccggcaatgcccatgccttacagacaggcgatttccgcacaccggccgaattggctttaaataaatttgagaaagaaagcggccatatccgcaatcagcgcagcgattattcgcatacgttcagccgcaaagatttacaggcggagctgattttgctgtttgaaaaacaaaaagaatttggcaatccgcatgtttcaggcggccttaaagaaggtattgaaaccctactgatgacgcaacgccctgccctgtccggcgatgccgttcaaaaaatgttggggcattgcaccttcgaaccggcagagccgaaagccgctaaaaacacctacacagccgaacgtttcatctggctgaccaagctgaacaacctgcgtattttagagcaaggcagcgagcggccattgaccgataccgaacgcgccacgcttatggacgagccatacagaaaatccaaactgacttacgcacaagcccgtaagctgctgggtttagaagataccgcctttttcaaaggcttgcgctatggtaaagacaatgccgaagcctcaacattgatggaaatgaaggcctaccatgccatcagccgtgcactggaaaaagaaggattgaaagacaaaaaatccccattaaacctttctcccgaattacaagacgaaatcggcacggcattctccctgttcaaaaccgatgaagacattacaggccgtctgaaagaccgtatacagcccgaaatcttagaagcgctgttgaaacacatcagcttcgataagttcgtccaaatttccttgaaagcattgcgccgaattgtgcctctaatggaacaaggcaaacgttacgatgaagcctgcgccgaaatctacggagaccattacggcaagaagaatacggaagaaaagatttatctgccgccgattcccgccgacgaaatccgcaaccccgtcgtcttgcgcgccttatctcaagcacgtaaggtcattaacggcgtggtacgccgttacggctccccagctcgtatccatattgaaactgcaagggaagtaggtaaatcgtttaaagaccgcaaagaaattgagaaacgccaagaagaaaaccgcaaagaccgggaaaaagccgccgccaaattccgagagtatttccccaattttgtcggagaacccaaatccaaagatattctgaaactgcgcctgtacgagcaacaacacggcaaatgcctgtattcgggcaaagaaatcaacttaggccgtctgaacgaaaaaggctatgtcgaaatcgaccatgccctgccgttctcgcgcacatgggacgacagtttcaacaataaagtactggtattgggcagcgaaaaccaaaacaaaggcaatcaaaccccttacgaatacttcaacggcaaagacaacagccgcgaatggcaggaatttaaagcgcgtgtcgaaaccagccgtttcccgcgcagtaaaaaacaacggattctgctgcaaaaattcgatgaagacggctttaaagaacgcaatctgaacgacacgcgctacgtcaaccgtttcctgtgtcaatttgttgccgaccgtatgcggctgacaggtaaaggcaagaaacgtgtctttgcatccaacggacaaattaccaatctgttgcgcggcttttggggattgcgcaaagtgcgtgcggaaaacgaccgccatcacgccttggacgccgtcgtcgttgcctgctcgaccgttgccatgcagcagaaaattacccgttttgtacgctataaagagatgaacgcgtttgacggtaaaaccatagacaaagaaacaggagaagtgctgcatcaaaaaacacacttcccacaaccttgggaatttttcgcacaagaagtcatgattcgcgtcttcggcaaaccggacggcaaacccgaattcgaagaagccgataccctagaaaaactgcgcacgttgcttgccgaaaaattatcatctcgccccgaagccgtacacgaatacgttacgccactgtttgtttcacgcgcgcccaatcggaagatgagcgggcaagggcatatggagaccgtcaaatccgccaaacgactggacgaaggcgtcagcgtgttgcgcgtaccgctgacacagttaaaactgaaagacttggaaaaaatggtcaatcgggagcgcgaacctaagctatacgaagcactgaaagcacggctggaagcacataaagacgatcctgccaaagcctttgccgagccgttttacaaatacgataaagcaggcaaccgcacccaacaggtaaaagccgtacgcgtagagcaagtacagaaaaccggcgtatgggtgcgcaaccataacggtattgccgacaacgcaaccatggtgcgcgtagatgtgtttgagaaaggcgacaagtattatctggtaccgatttacagttggcaggtagcgaaagggattttgccggatagggctgttgtacaaggaaaagatgaagaagattggcaacttattgatgatagtttcaactttaaattctcattacaccctaatgatttagtcgaggttataacaaaaaaagctagaatgtttggttactttgccagctgccatcgaggcacaggtaatatcaatatacgcattcatgatcttgatcataaaattggcaaaaatggaatactggaaggtatcggcgtcaaaaccgccctttcattccaaaaataccaaattgacgaactgggcaaagaaatcagaccatgccgtctgaaaaaacgcccgcctgtccgtgattataaggatgatgatgataagtaa

SEQ ID NO: 34 - DNA, 3345 bp, synthetic construct
  source 1..3345: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaa

SEQ ID NO: 35 - DNA, 3051 bp, synthetic construct
  source 1..3051: mol_type = other DNA
atggactataaggacgacgacgataagataatggcccctaagaagaaaagaaaggtgggatccatggcccgcattcttgctttcgatattggcatcagctcaataggatgggcattttccgagaatgacgaattgaaggactgcggagttaggatcttcactaaagtagagaaccctaagactggtgaatccctggcactgcccagaagactggctaggagcgcacggaagcgactcgctcgcagaaaggcaagacttaatcaccttaaacaccttattgcaaacgagttcaagctcaattacgaagattatcagtcttttgacgaaagccttgccaaggcctacaaaggtagtctcatcagcccctacgaactcaggttcagggcactgaatgaactcctgtccaagcaggatttcgcaagagtcatacttcacatagctaaacggaggggctatgatgacataaagaactccgacgacaaggaaaaaggggctatacttaaggcaatcaaacagaacgaagagaaacttgctaactaccaatcagtaggagaatacctgtataaggaatattttcagaagtttaaggaaaactccaaggaattcactaacgttcgaaacaagaaggagtcttatgaacgctgcatcgcacagagtttcttgaaggatgagctgaaacttatattcaaaaagcaaagggagttcggattctcctttagcaaaaaatttgaagaggaggtgttgagcgtcgctttttacaaacgggcactcaaagacttttcacatctggttggcaattgctctttctttaccgatgagaaacgcgtacctaagaatagtccactcgcttttatgtttgtcgctttgacacgaattataaacctgctgaataaccttaaaaatacagagggcatcctgtacacaaaggatgatctcaatgctctgttgaatgaagttctcaagaacggtactctgacttataaacaaacaaagaagctgctcggtctgtcagacgattatgagtttaagggcgaaaaggggacctactttatcgagtttaagaagtataaagaattcatcaaagccctgggagagcataacttgtcacaagatgatcttaatgagattgcaaaagatataaccctcataaaggatgagattaaactgaaaaaggccctcgccaagtacgatcttaatcagaaccaaattgactcattgagcaagctggaattcaaagatcacctgaacatttcatttaaggccctcaagcttattacacccttgatgctggaaggcaaaaaatatgacgaagcttgcaacgagctcaatctgaaagtcgccataaacgaggataaaaaagatttccttcccgcatttaacgagacctattacaaggatgaagtgactaatcccgtggttcttcgagcaataaaggaatacaggaaagttcttaatgcactcctcaaaaaatatggcaaagtacataaaattaacatagagctcgcccgagaggtaggaaagaatcattcccaaagggcaaaaatcgaaaaagagcaaaatgaaaattacaaggcaaagaaggatgccgagctcgagtgcgaaaaactggggctgaagatcaattccaagaacatattgaagctgcgactcttcaaggaacagaaggaattttgtgcttacagcggagaaaaaataaaaataagcgatctgcaagatgaaaagatgctcgagatcgaccacatctacccatattcaagatctttcgacgactcctacatgaataaagtcctcgtttttaccaaacagaatcaagagaagctcaaccagaccccctttgaggccttcggcaacgacagtgcaaagtggcaaaaaatagaagtcctcgctaaaaatctccccacaaaaaaacaaaagaggatattggacaagaactataaagataaggagcaaaaaaactttaaagacaggaatcttaacgacactagatacatcgctagactggtattgaattataccaaagactacctggactttcttccactgagcgacgacgaaaacacaaaactcaacgacacacagaaaggtagtaaggtacatgttgaggccaaaagtggaatgttgacttcagcccttcgacatacctggggtttttctgctaaagatcgcaataaccaccttcaccatgccattgatgctgttattattgcatacgctaacaactcaatagttaaggctttctctgattttaagaaagaacaggaaagcaattctgctgagctctatgcaaaaaagatcagcgagttggattacaagaacaagaggaaattttttgagcccttttccggattcagacaaaaggtcttggacaaaattgatgagatttttgtgtcaaagcctgagagaaagaagcctagtggagccctgcacgaggaaacctttaggaaggaggaggaattttatcaaagttacggtggtaaagagggtgttcttaaagcactcgagcttggaaagatccggaaggtgaacgggaagatagtcaaaaatggggacatgttccgggtcgatatttttaagcacaagaagacaaacaagttctacggtgtacccatatacaccatggattttgctcttaaagtattgccaaataaggctgtggctagaagcaagaaaggagaaatcaaggactggatactcatggatgaaaactacgaattctgcttctcactttacaaggactccctcattctcatccaaaccaaagatatgcaggaacctgagttcgtttactataatgcttttaccagctctaccgtgtcccttatcgtctccaagcacgacaataaattcgaaacactgtctaagaatcaaaaaattttgtttaagaatgctaacgagaaggaggtgattgcaaagagcattggcattcaaaacctgaaagtgtttgaaaaatacatcgtttcagcactgggagaagtaacaaaggctgaatttaggcaaagagaggacttcaagaaaggatccatggctcctaagaaaaaacggaaggtttga

SEQ ID NO: 36 - DNA, 4161 bp, synthetic construct
  source 1..4161: mol_type = other DNA
atggaaaaaaaatactcaatcggattggatatcggcactaactcagttggatgggctgtaattacagacgattacaaggtccctagcaaaaagtttaaggtgctgggtaatacgaatcgcaagagtattaagaagaatctgatgggtgcattactgtttgattctggcgaaacagccgaagctactcgtttaaagcgcaccgcccgccgtcgttatacacgccgcaagaatcgcattcgctatcttcaagaaatcttcgctaatgaaatggcgaagctggacgactctttcttccagcgtttggaggaaagttttcttgtggaggaggacaaaaaaaatgaacgtcaccccattttcgggaacttagcggacgaggtcgcctatcaccgtaactatcccactatttaccacctgcgcaaaaagcttgcggattctccagaaaaggctgaccttcgcctgatttatctggcattggcacatatcatcaaatttcgtgggcacttccttattgaaggaaaattgaacgcagaaaattctgatgtcgcaaaactgttctaccagttgatccagacatataatcaattattcgaggagagtcccctggatgaaatcgaagtggatgcaaagggtatcctgtccgcccgtctgtcaaagagtaaacgtcttgaaaagctgattgcggtgtttcccaatgaaaaaaagaacggtttgttcgggaacattatcgctctggcccttggtttaacccctaactttaagtcaaactttgacctgactgaggacgcgaaattacagctttctaaagatacttatgacgatgacttagatgagcttctggggcaaattggggaccagtacgccgatcttttcagtgcagccaagaatcttagtgacgcgattttgctttcagacatcttacgttccaacagcgaagtaacaaaggcgccactgagcgcctctatggttaagcgttatgacgagcaccatcaggaccttgcacttcttaagactctggtgcgtcaacagtttccagaaaagtacgcagagatcttcaaggacgatacaaaaaacggatacgccgggtatgtcggaattggaattaaacatcgcaagcgcacaactaaactggcgacacaagaagagttctacaaattcattaaacctattcttgagaaaatggacggggccgaggagctgttggccaaactgaaccgtgacgaccttttacgtaaacagcgcacctttgacaacggttccattcctcaccagatccatcttaaagaattgcacgcaattttgcgtcgtcaggaggagttttatccatttctgaaagagaatcgtgagaaaattgagaaaatccttacgttccgcatcccctactatgttggtcctctggctcgcggcaactcccgcttcgcgtggttgacacgtaagtctgaggaggctatcactccctggaacttcgaggaagtggtggacaaaggagccagcgcacagagctttatcgaacgtatgacaaatttcgacgaacagcttccaaacaaaaaagtcttaccgaagcatagtcttttatatgagtatttcaccgtctataatgagttaacgaaagtgaagtacgttactgagcgcatgcgtaagcccgagtttttaagtggcgagcaaaaaaaggctattgtcgatcttctgtttaagactaaccgcaaagtcaccgtgaaacaacttaaagaagattattttaagaagatcgaatgtttcgattcggtagaaattattggcgtagaggatcgtttcaacgctagtttggggacataccatgacttacttaagatcatcaaggacaaggattttttggataatgaagaaaacgaggatattctggaggacattgtattgaccctgacactgttcgaggaccgtgaaatgattgaagagcgtttaaaaacatatgcgcacctgtttgacgataaagtgatgaagcaattaaagcgccgtcattacactgggtggggtcgtctttctcgtaaaatgattaacggtatccgtgataaacagtctggtaagacgattctggacttccttaagagcgatggcttctcgaaccgtaactttatgcaactgattcacgatgactccctgacgtttaaagaagaaattgagaaggctcaagtctctgggcaaggggacagtctgcacgagcaaattgctgaccttgccggaagtcccgcgattaagaaaggaatcttgcaaacagtaaaaattgtggatgaattggttaaagtgatgggacataaacccgaaaatattgtcatcgagatggcgcgcgagaaccagaccactaccaaggggcttcaacaatcgcgtgaacgtaagaaacgcatcgaagaaggcattaaggagttggagagccaaattctgaaagaaaatccagtagagaatactcagttacagaatgaaaaattgtatctttattacttacagaacggacgcgatatgtacgtggatcaagagctggacatcaatcgcctgagtgactacgacgtagatcatatcgttcctcagtcgtttatcaaggatgacagcatcgacaataaagtgctgacgcgtagtgtcgaaaatcgcggaaaatcggataatgtgccatcggaggaagtcgtgaaaaaaatgaagaattattggcgccaacttttgaacgccaagttgattacccaacgtaaattcgataatcttaccaaggctgaacgcgggggcctgtccgaagcagacaaagcagggtttattaagcgtcaacttgtagagacacgtcagattacaaagcatgtcgcccgcattcttgattcgcgcatgaatactaagcgtgataaaaatgacaagcccattcgtgaggttaaagtaattacactgaagtctaagctggttagtgactttcgtaaagacttccagttatacaaggtgcgcgatatcaataattatcatcacgcccatgacgcttatctgaatgcggttgtaggtacggccctgatcaagaagtatccaaaattagagtccgagttcgtgtacggtgattacaaggtctacgatgtccgtaaaatgattgccaaatccgaacaagaaatcggtaaagcgactgcaaaacgcttcttctattctaacattatgaatttctttaagaccgaggtaaaattggcgaacggggaaattcgtaaacgtccgttgatcgagacaaacggagaaacgggcgaggttgtttggaacaaagaaaaagacttcgccactgttcgtaaagttctggcgatgccgcaggtaaacatcgtaaagaaaaccgaagttcaaactggaggattttctaaagaatcaatcctttctaaacgtgaatcggcgaagttgattccgcgcaaaaaagggtgggacactcgtaaatacggcggttttgggagtccgactgtagcctatagtatcctggtggttgcaaaggtcgagaagggaaaagctaaaaaactgaaatccgttaaggtcttggttgggatcacgattatggagaaaggaagctatgaaaaggaccctattggttttctggaagccaaaggctacaaggatatcaaaaaggagttgatcttcaaattacctaaatattcattgttcgaactggagaacggacgccgtcgtatgttagcttctgcaaccgagttgcagaaggctaacgaactggtattgccacaacatctggtgcgtttactttattatacacagaacatctcggctacgacaggctctaacaatttggggtacatcgagcaacatcgtgaagaattcaaggagattttcgaaaaaattattgatttctcggagaagtatatcttgaaaaataaagtgaattcaaacttgaaatcgagttttgacgagcaattcgcggtatcggattccattctgttgtccaacagctttgttagcctgttgaaatatacctcttttggcgcgtcgggaggattcaccttcctggaccttgacgttaagcaagggcgcttgcgctaccagacggttacggaggttctggatgcgaccttgatctatcaaagcatcaccggactgtacgagacacgtacagatctgagccaactgggaggagactcgcgggctgacccgaaaaagaagagaaaggtctag

SEQ ID NO: 37 - DNA, 4221 bp, synthetic construct
  source 1..4221: mol_type = other DNA
atggagaagaaatactccatcggcctggacatcggaaccaacagcgtgggctgggccgtgatcaccgacgactataaggtgcccagcaagaagttcaaagtgctgggcaacacaaacagaaagagcatcaagaagaacctgatgggcgccctgctgttcgactccggagagaccgccgaagcaacccggctgaagcgcaccgccagaagacggtacaccagaagaaaaaacaggatcagatacctgcaggagatcttcgccaacgagatggccaaactggacgatagctttttccagcggctggaagaatccttcctggtggaggaggacaagaaaaacgagagacaccctatctttgggaacctggccgacgaggtggcctaccacagaaactaccccaccatctaccacctgagaaagaagctggccgactcccccgaaaaggccgacctgagactgatctacctcgccctggcccacatcatcaagtttcggggccacttcctgattgaaggcaagctgaacgccgagaactccgacgtggccaagctgttctaccagctgatccagacctacaatcagctgtttgaggaaagccctctggacgaaatcgaagtggacgccaaagggatcctgtccgcccggctgagcaagtctaagcggctggagaagctgattgccgtcttccccaacgagaagaaaaacggcctgttcggcaacattatcgccctggccctgggcctgacccccaactttaagagcaatttcgatctgaccgaggatgccaagctgcagctgtccaaggatacctacgacgacgatctggacgagctgctgggacagatcggagaccagtacgccgacctgttcagcgccgccaaaaacctgtccgatgccatcctgctgagcgacatcctgcggagcaacagcgaagtcaccaaagcgcccctgagcgccagcatggtgaagcggtacgacgagcaccaccaggacctggcgctgctgaagacactggtgagacagcagttccccgagaagtacgccgagatcttcaaggacgataccaaaaatggctacgctggctacgtgggagctgataagaagctgcggaagcggtccggcaagctggccaccgaggaggagttttacaagttcatcaagccgatcctggaaaagatggatggagccgaagagctgctggccaagctgaaccgggacgacctgctgagaaagcaacggaccttcgacaacggatccatccctcaccagatccacctgaaagagctgcacgctatcctgagacggcaagaagaattttaccccttcctgaaggagaaccgggagaagatcgaaaagatcctgaccttcagaatcccctactacgtggggccactggcccggggcaatagcagattcgcctggctgacccggaagtctgaagaggccatcaccccctggaattttgaggaggtggtggacaaaggcgccagcgcccagtccttcatcgagcggatgaccaacttcgatgagcagctgccaaacaagaaggtcctgcccaagcactctctgctgtacgaatattttaccgtgtacaacgaactgaccaaagtgaagtacgtgactgagagaatgcggaagcctgaattcctgtccggagagcagaagaaagccatcgtggacctgctgttcaagaccaacagaaaagtgaccgtgaaacagctgaaggaggactacttcaagaagatcgagtgcttcgacagcgtggagattatcggagtggaagacagattcaacgccagcctgggcacctaccacgatctgctgaagatcatcaaggataaggacttcctggacaacgaggagaacgaagacatcctggaggacatcgtgctgacactgaccctgttcgaggaccgggaaatgatcgaagaacggctcaaaacatacgctcacctgttcgatgacaaggtgatgaagcagctgaagagaagacactacaccggctggggccggctgtcccggaagatgatcaacggaatccgggacaagcagtccggcaagacaatcctggacttcctgaagtccgacggcttcagcaacagaaacttcatgcagctgatccacgacgacagcctgacctttaaagaggagatcgagaaagcccaggtgtccggccaaggagactctctgcacgagcagatcgccgacctggccggcagccccgccatcaaaaaaggcattctgcagaccgtgaagatcgtggatgagctggtgaaagtgatgggccacaagcctgagaacatcgtgatcgagatggccagagagaaccagaccaccaccaaaggactgcagcagtccagggagcggaaaaagcggatcgaggaaggcatcaaggagctggaaagccagattctgaaggaaaaccccgtggaaaacacccagctgcagaacgaaaagctgtacctgtactacctgcagaatggcagagatatgtacgtggatcaggagctggatatcaatcggctgtccgactacgatgtggaccacatcgtgccccagagcttcatcaaggacgactccatcgataacaaagtgctgacccggtctgtggagaacagaggcaagagcgacaacgtgcccagcgaagaagtggtgaagaagatgaagaactactggcggcagctgctgaacgccaagctgatcacccagagaaagttcgacaacctgaccaaggccgagcggggcggcctgagcgaagccgataaggctggcttcatcaagcggcagctggtggaaaccaggcagattacaaagcacgtggcccggatcctggacagcagaatgaacaccaagagagacaagaacgacaagcccatccgggaggtgaaggtgatcaccctgaagagcaaactggtgagcgacttcagaaaagacttccagctgtacaaagtgcgggacatcaacaactaccaccacgcccacgacgcctacctgaacgccgtggtgggcaccgctctgatcaagaagtaccccaagctggagagcgagttcgtgtacggcgattataaggtgtacgatgtgcggaaaatgatcgccaagagcgagcaggagatcggcaaggccaccgccaaacggttcttctacagcaacatcatgaatttcttcaagaccgaggtgaagctggccaacggcgagatcagaaagagacctctgattgaaaccaacggcgagaccggcgaagtggtgtggaacaaggagaaggacttcgccaccgtgcggaaggtgctggccatgccccaggtgaacatcgtgaagaagaccgaggtgcagactggcgggttctctaaggaatctatcctgagcaagcgggagagcgccaaactgatcccccggaagaagggctgggacaccagaaagtacggcggattcggcagcccaaccgtggcctacagcatcctggtggtggccaaggtggagaagggcaaggccaaaaagctgaaaagcgtgaaagtgctggtgggaatcaccatcatggaaaagggcagctacgagaaggaccccattggattcctggaagcaaagggctacaaggacatcaagaaggagctgatctttaaactgccgaagtacagcctgttcgaactggagaacggccggagacggatgctggccagcgccaaggagctgcagaaggccaatgagctggtgctgccccagcacctggtgagactgctgtactacacccagaacatcagcgccaccaccggcagcaacaacctgggctatatcgagcagcaccgggaggaattcaaagagatcttcgaaaagatcatcgatttcagcgagaagtacatcctgaagaacaaggtgaacagcaacctgaagagcagtttcgatgaacagttcgcagtcagcgactccatcctgctgagcaattccttcgtgtccctgctgaaatacacctccttcggagcctccggcggcttcaccttcctggacctggacgtgaagcagggccggctgcggtaccagaccgtgaccgaagtgctggatgccaccctgatctaccagtccatcaccggcctgtacgagacccggaccgacctgagccagctgggcggagacagcggcggaagcggaggcagcggcggaagcggaggcagcaagagaaccgccgatggaagcgagttcgagtctcccaagaagaagaggaaagtctaa

SEQ ID NO: 38-40 - skipped (3 entries)

SEQ ID NO: 41 - protein, 1385 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDKRPAATKKAGQAKKKKK

SEQ ID NO: 42 - protein, 1385 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKK

SEQ ID NO: 43 - protein, 1385 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDKRPAATKKAGQAKKKKK

SEQ ID NO: 44 - protein, 1385 aa, synthetic construct
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDKRPAATKKAGQAKKKKK

SEQ ID NO: 45 - protein, 1385 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKK

SEQ ID NO: 46 - protein, 1385 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKK

SEQ ID NO: 47 - protein, 1323 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLALAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNREKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKK

SEQ ID NO: 48 - protein, 572 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIE

SEQ ID NO: 49 - protein, 812 aa, synthetic construct
CFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKK

SEQ ID NO: 50 - protein, 1090 aa, synthetic construct
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRDYKDDDDK

SEQ ID NO: 51 - protein, 1114 aa, synthetic construct
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSYPYDVPDYAYPYDVPDYAYPYDVPDYA

SEQ ID NO: 52 - protein, 1016 aa, synthetic construct
MDYKDDDDKIMAPKKKRKVGSMARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRVPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYGVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKKGSMAPKKKRKV

SEQ ID NO: 53 - protein, 1386 aa, synthetic construct
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSRADPKKKRKV

SEQ ID NO: 54 - protein, 1406 aa, synthetic construct
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGADKKLRKRSGKLATEEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASAKELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSGGSGGSGGSKRTADGSEFESPKKKRKV

SEQ ID NO: 55 - protein, 1228 aa, unidentified
  source 1..1228: note = Lachnospiraceae bacterium sequence
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 56 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 57 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 58-60 - skipped (3 entries)

SEQ ID NO: 61 - DNA, 5107 bp, synthetic construct
  source 1..5107: mol_type = other DNA
taatacgactcactataggggaattgtgagcggataacaattcccctctagaaataattttgtttaactttaagaaggagatatacctgataatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagctcccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 62 - DNA, 4083 bp, synthetic construct
  source 1..4083: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagacccctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 63 - DNA, 4084 bp, synthetic construct
  source 1..4084: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagacccctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 64 - DNA, 4099 bp, synthetic construct
  source 1..4099: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgtcataaaaaatttatttgctttgtgagcggataacaattataatatgtggaattgtgagcggataacaatttcacacaggaaacagacccctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 65 - DNA, 4072 bp, synthetic construct
  source 1..4072: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgtaatacgactcactatagtgtggaattgtgagcggataacaatttcacacaggaaacagacccctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 66 - DNA, 4097 bp, synthetic construct
  source 1..4097: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgtaatacgactcactatagttgtgagcggataacaattataatatgtggaattgtgagcggataacaatttcacacaggaaacagacccctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 67 - DNA, 5238 bp, synthetic construct
  source 1..5238: mol_type = other DNA
atgggtatccagggtctgctgcagttcatcaaagaagcttctgaaccgatccacgttcgtaaatacaaaggtcaggttgttgctgttgacacctactgctggctgcacaaaggtgctatcgcttgcgctgaaaaactggctaaaggtgaaccgaccgaccgttacgtaggcttctgcatgaaatttgttaacatgctgctgtctcacggtatcaaaccgatcctggttttcgacggttgcaccctgccgtctaaaaaagaagttgaacgttctcgtcgtgaacgtcgtcaggctaacctgctgaaaggtaaacagctgctgcgtgaaggtaaagtttctgaagctcgtgaatgcttcacccgttctatcaacatcacccacgctatggctcacaaagttatcaaagctgctcgttctcagggtgttgactgcctggttgctccgtacgaagctgacgctcagctggcttacctgaacaaagctggtatcgttcaggctatcatcaccgaagactctgacctgctggctttcggttgcaaaaaagttatcctgaaaatggaccagttcggtaacggtctggaaatcgaccaggctcgtctgggtatgtgccgtcagctcggcgacgtcttcaccgaagaaaaattccgttacatgtgcatcctgtctggttgcgactacctgtcttctctgcgtggtatcggtctggctaaagcttgcaaagttctgcgtctggctaacaacccggacatcgttaaagttatcaaaaaaatcggtcactacctgaaaatgaacatcaccgttccggaagactacatcaacggtttcatccgtgctaacaacaccttcctgtaccagctggttttcgacccgatcaaacgtaaactgatcccgctgaacgcttacgaagacgacgttgacccggaaaccctgtcttacgctggtcagtacgttgacgactctatcgctctgcagatcgctctgggtaacaaagacatcaacaccttcgaacagatcgacgactacaacccggacaccgctatgccggctcactctcgtgctccggctccggctccggctccggctccggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgaccccaagaagaagaggaaggtgatggataagcatcaccaccaccatcactaa

SEQ ID NO: 68 - DNA, 5223 bp, synthetic construct
  source 1..5223: mol_type = other DNA
atgggtatccagggtctgctgcagttcatcaaagaagcttctgaaccgatccacgttcgtaaatacaaaggtcaggttgttgctgttgacacctactgctggctgcacaaaggtgctatcgcttgcgctgaaaaactggctaaaggtgaaccgaccgaccgttacgtaggcttctgcatgaaatttgttaacatgctgctgtctcacggtatcaaaccgatcctggttttcgacggttgcaccctgccgtctaaaaaagaagttgaacgttctcgtcgtgaacgtcgtcaggctaacctgctgaaaggtaaacagctgctgcgtgaaggtaaagtttctgaagctcgtgaatgcttcacccgttctatcaacatcacccacgctatggctcacaaagttatcaaagctgctcgttctcagggtgttgactgcctggttgctccgtacgaagctgacgctcagctggcttacctgaacaaagctggtatcgttcaggctatcatcaccgaagactctgacctgctggctttcggttgcaaaaaagttatcctgaaaatggaccagttcggtaacggtctggaaatcgaccaggctcgtctgggtatgtgccgtcagctcggcgacgtcttcaccgaagaaaaattccgttacatgtgcatcctgtctggttgcgactacctgtcttctctgcgtggtatcggtctggctaaagcttgcaaagttctgcgtctggctaacaacccggacatcgttaaagttatcaaaaaaatcggtcactacctgaaaatgaacatcaccgttccggaagactacatcaacggtttcatccgtgctaacaacaccttcctgtaccagctggttttcgacccgatcaaacgtaaactgatcccgctgaacgcttacgaagacgacgttgacccggaaaccctgtcttacgctggtcagtacgttgacgactctatcgctctgcagatcgctctgggtaacaaagacatcaacaccttcgaacagatcgacgactacaacccggacaccgctatgccggctcactctcgtggtggtggtggttctgataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgaccccaagaagaagaggaaggtgatggataagcatcaccaccaccatcactaa

SEQ ID NO: 69 - DNA, 5355 bp, synthetic construct
  source 1..5355: mol_type = other DNA
atgggcatccaaggccttttacagtttattaaagaggcttcggagcctattcatgtccgtaagtacaagggacaagttgttgcagttgatacatattgctggcttcataagggggctattgcctgcgcagaaaagctggctaagggcgagccaacggaccgctacgtgggcttttgtatgaagttcgtcaatatgcttttgtcccatggcatcaaacctattttggtctttgacggatgtactttgccttcaaagaaagaggtagaacgttcgcgccgcgaacgtcgccaagcaaatctgttgaaaggtaagcagttgcttcgcgagggcaaggtctcagaagctcgtgaatgctttacccgctctattaacatcacgcatgcgatggcgcacaaggtcatcaaagcagcccgctcgcaaggagtcgattgcctggtcgcgccctatgaggcggatgcccaactggcttaccttaacaaggcagggatcgttcaggcgattatcaccgaagattccgatttattggccttcggctgcaaaaaagtgatcttgaaaatggatcaatttggcaatggattagaaattgaccaagcacgcctgggaatgtgtcgtcaactgggcgatgttttcaccgaggaaaagttccgctacatgtgcattctttcaggttgcgactacttgagtagccttcgtgggatcggtcttgcgaaggcttgcaaagttctgcgccttgctaacaacccagacatcgtaaaggttatcaaaaaaatcggacattacttaaagatgaatattaccgtaccggaggattacatcaatgggtttatccgcgcaaacaacacattcctttatcagcttgtcttcgatccaattaagcgtaaacttatcccgttgaatgcatatgaggacgatgtggaccccgaaacattatcctatgcaggccaatacgtagacgacagcatcgcgctgcagattgctttaggaaacaaagacatcaatacgttcgagcagattgatgattataacccggacactgcgatgccagcacattcccgcgcgccagctcctgctccagccccggccccggattacaaggatcatgatggcgactataaggatcatgacatcgattataaggacgatgacgacaagatggcgcctaaaaaaaagcgcaaagtaggcatccacggagttcctgcggccgataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgaccccaagaagaagaggaaggtgatggataagcatcaccaccaccatcactaa

SEQ ID NO: 70 - skipped

SEQ ID NO: 71 - DNA, 4237 bp, synthetic construct
  source 1..4237: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccgaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 72 - DNA, 4837 bp, synthetic construct
  source 1..4837: mol_type = other DNA
ggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggcccgggatccaccggtcgccacctaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 73 - DNA, 3143 bp, synthetic construct
  source 1..3143: mol_type = other DNA
ggtcctgtctcatcattttggcaaagaattctgcagtcgacggtaccgcgggcccgggatccaccggtcgccacctaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 74 - DNA, 3762 bp, synthetic construct
  source 1..3762: mol_type = other DNA
ctagtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccacctaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattga

SEQ ID NO: 75-80 - skipped (6 entries)

SEQ ID NO: 81 - DNA, 6213 bp, synthetic construct
  source 1..6213: mol_type = other DNA
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagagcacctgctccagcgcccgcaccagctcccgactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaaacaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcaataccgcagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaaggctccggcgagggcaggggaagtcttctaacatgcggggacgtggaggaaaatcccggcccaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctaa

SEQ ID NO: 82 - DNA, 6033 bp, synthetic construct
  source 1..6033: mol_type = other DNA
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagagcacctgctccagcgcccgcaccagctcccgactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaaacaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcaataccgcagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaaggctccggcgagggcaggggaagtcttctaacatgcggggacgtggaggaaaatcccggcccaaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcgggatccaccggatctagataa

SEQ ID NO: 83 - DNA, 5358 bp, synthetic construct
  source 1..5358: mol_type = other DNA
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagagcacctgctccagcgcccgcaccagctcccgactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaaacaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcaataccgcagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaataa

SEQ ID NO: 84 - DNA, 5376 bp, synthetic construct
  source 1..5376: mol_type = other DNA
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagagcacctgctccagcgcccgcaccagctcccgactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaaacaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcaataccgcagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaacatcaccaccaccatcactaa

SEQ ID NO: 85 - DNA, 6231 bp, synthetic construct
  source 1..6231: mol_type = other DNA
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagagcacctgctccagcgcccgcaccagctcccgactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaaacaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcaataccgcagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaacatcaccaccaccatcacggctccggcgagggcaggggaagtcttctaacatgcggggacgtggaggaaaatcccggcccaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctaa

SEQ ID NO: 86 - DNA, 6051 bp, synthetic construct
  source 1..6051: mol_type = other DNA
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagagcacctgctccagcgcccgcaccagctcccgactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaaacaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcaataccgcagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagaaacatcaccaccaccatcacggctccggcgagggcaggggaagtcttctaacatgcggggacgtggaggaaaatcccggcccaaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcgggatccaccggatctagataa

SEQ ID NO: 87-90 - skipped (4 entries)

SEQ ID NO: 91 - protein, 2068 aa, synthetic construct
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRAPAPAPAPAPDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDKRPAATKKAGQAKKKKKGSGEGRGSLLTCGDVEENPGPMIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLLYGIAAPDSQRIAFYRLLDEFF

SEQ ID NO: 92 - protein, 2010 aa, synthetic construct
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRAPAPAPAPAPDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDKRPAATKKAGQAKKKKKGSGEGRGSLLTCGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSTGSR

SEQ ID NO: 93 - protein, 1785 aa, synthetic construct
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRAPAPAPAPAPDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDKRPAATKKAGQAKKKKK

SEQ ID NO: 94 - protein, 1791 aa, synthetic construct
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRAPAPAPAPAPDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDKRPAATKKAGQAKKKKKHHHHHH

SEQ ID NO: 95 - protein, 2076 aa, synthetic construct
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRAPAPAPAPAPDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDKRPAATKKAGQAKKKKKHHHHHHGSGEGRGSLLTCGDVEENPGPMIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 96 - protein, 2016 aa, synthetic construct
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRAPAPAPAPAPDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGDKRPAATKKAGQAKKKKKHHHHHHGSGEGRGSLLTCGDVEENPGPTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGSTGSR

SEQ ID NO: 97-100 - skipped (4 entries)

SEQ ID NO: 101 - DNA, 17 bp, synthetic construct
  source 1..17: mol_type = other DNA
gggtcttcgagaagacc

SEQ ID NO: 102 - DNA, 20 bp, synthetic construct
  source 1..20: mol_type = other DNA
ggagtgcaatggcgcgatct

SEQ ID NO: 103 - DNA, 20 bp, synthetic construct
  source 1..20: mol_type = other DNA
gcgccattgcactccagcct

SEQ ID NO: 104 - DNA, 20 bp, synthetic construct
  source 1..20: mol_type = other DNA
gtaacggcagacttctcctc

SEQ ID NO: 105 - DNA, 20 bp, synthetic construct
  source 1..20: mol_type = other DNA
gtctgccgttactgccctgt

SEQ ID NO: 106 - DNA, 20 bp, synthetic construct
  source 1..20: mol_type = other DNA
gaggtgaacgtggatgaagt

SEQ ID NO: 107 - DNA, 20 bp, synthetic construct
  source 1..20: mol_type = other DNA
ttatttagagctagtgtact

SEQ ID NO: 108 - DNA, 20 bp, synthetic construct
  source 1..20: mol_type = other DNA
gtcccctccaccccacagtg

SEQ ID NO: 109 - DNA, 20 bp, synthetic construct
  source 1..20: mol_type = other DNA
ggggccactagggacaggat

SEQ ID NO: 110 - DNA, 21 bp, synthetic construct
  source 1..21: mol_type = other DNA
gacgactaggggactcgagag

SEQ ID NO: 111 - DNA, 19 bp, synthetic construct
  source 1..19: mol_type = other DNA
gaggcggcgaggacgacta

SEQ ID NO: 112 - DNA, 21 bp, synthetic construct
  source 1..21: mol_type = other DNA
gttacaggtcagtatcaaacc

SEQ ID NO: 113 - DNA, 21 bp, synthetic construct
  source 1..21: mol_type = other DNA
gtccaggtgacagcaatcagc

SEQ ID NO: 114 - DNA, 21 bp, synthetic construct
  source 1..21: mol_type = other DNA
gtgacctgtaaatcatccttt

SEQ ID NO: 115 - DNA, 21 bp, synthetic construct
  source 1..21: mol_type = other DNA
gtttagctcttgggccctatc

SEQ ID NO: 116 - DNA, 21 bp, synthetic construct
  source 1..21: mol_type = other DNA
gaagatgaccctttcccagat

SEQ ID NO: 117 - DNA, 21 bp, synthetic construct
  source 1..21: mol_type = other DNA
gtcttgggccctatctgggaa

SEQ ID NO: 118-120 - skipped (3 entries)

SEQ ID NO: 121 - DNA, 20 bp, synthetic construct
  source 1..20: mol_type = other DNA
ggcatccaccctaggtacaa

SEQ ID NO: 122 - DNA, 20 bp, synthetic construct
  source 1..20: mol_type = other DNA
cgaggcagtagaatcgcttg

SEQ ID NO: 123 - DNA, 20 bp, synthetic construct
  source 1..20: mol_type = other DNA
cactaaggcgcagaagaagg

SEQ ID NO: 124 - DNA, 19 bp, synthetic construct
  source 1..19: mol_type = other DNA
gagccgagatcgcgccatg

SEQ ID NO: 125 - DNA, 21 bp, synthetic construct
  source 1..21: mol_type = other DNA
aacgatcctgagacttccaca

SEQ ID NO: 126 - DNA, 21 bp, synthetic construct
  source 1..21: mol_type = other DNA
tgcttaccaagctgtgattcc

SEQ ID NO: 127 - DNA, 20 bp, synthetic construct
  source 1..20: mol_type = other DNA
caaactgcaaggctgcaata

SEQ ID NO: 128 - DNA, 20 bp, synthetic construct
  source 1..20: mol_type = other DNA
gacccaccatgtcaaagtcc

SEQ ID NO: 129 - DNA, 20 bp, synthetic construct
  source 1..20: mol_type = other DNA
aggcctttaccgatgtgatg

SEQ ID NO: 130 - DNA, 20 bp, synthetic construct
  source 1..20: mol_type = other DNA
acggagtctcgctctgtcac

SEQ ID NO: 131 - DNA, 20 bp, synthetic construct
  source 1..20: mol_type = other DNA
acacggtgaaaccctgtctc

SEQ ID NO: 132 - DNA, 19 bp, synthetic construct
  source 1..19: mol_type = other DNA
acagatggaaggcctcctg

SEQ ID NO: 133 - DNA, 20 bp, synthetic construct
  source 1..20: mol_type = other DNA
cgggactatggttgctgact

SEQ ID NO: 134 - DNA, 23 bp, synthetic construct
  source 1..23: mol_type = other DNA
cccataattgataagccaaaaca

SEQ ID NO: 135 - DNA, 20 bp, synthetic construct
  source 1..20: mol_type = other DNA
tttctgtctgcagcttgtgg

SEQ ID NO: 136 - DNA, 20 bp, synthetic construct
  source 1..20: mol_type = other DNA
cttcctccactccctcttcc

SEQ ID NO: 137 - DNA, 24 bp, synthetic construct
  source 1..24: mol_type = other DNA
ccatttaccgtaagttatgtaacg

SEQ ID NO: 138 - DNA, 24 bp, synthetic construct
  source 1..24: mol_type = other DNA
cgttacataacttacggtaaatgg

SEQ ID NO: 139 - DNA, 20 bp, synthetic construct
  source 1..20: mol_type = other DNA
tggtggcaccggtccaacct

SEQ ID NO: 140 - DNA, 20 bp, synthetic construct
  source 1..20: mol_type = other DNA
aggttggaccggtgccacca

SEQ ID NO: 141 - DNA, 21 bp, synthetic construct
  source 1..21: mol_type = other DNA
ggatggggcttttctgtcacg

SEQ ID NO: 142 - DNA, 21 bp, synthetic construct
  source 1..21: mol_type = other DNA
cgtgacagaaaagccccatcc

SEQ ID NO: 143 - DNA, 20 bp, synthetic construct
  source 1..20: mol_type = other DNA
atgagatggtggacgaggaa

SEQ ID NO: 144 - DNA, 20 bp, synthetic construct
  source 1..20: mol_type = other DNA
agagccaggaacccctgtag

SEQ ID NO: 145 - DNA, 37 bp, synthetic construct
  source 1..37: mol_type = other DNA
gtcgactctagaggatccttcctccactccctcttcc

SEQ ID NO: 146 - DNA, 38 bp, synthetic construct
  source 1..38: mol_type = other DNA
gctcggtacccggggatcatgagatggtggacgaggaa

SEQ ID NO: 147 - DNA, 20 bp, synthetic construct
  source 1..20: mol_type = other DNA
ctgccgtctctctcctgagt

SEQ ID NO: 148 - DNA, 20 bp, synthetic construct
  source 1..20: mol_type = other DNA
tctaacgctgccgtctctct

SEQ ID NO: 149 - DNA, 20 bp, synthetic construct
  source 1..20: mol_type = other DNA
gtttacatctggcccaccac

SEQ ID NO: 150 - DNA, 20 bp, synthetic construct
  source 1..20: mol_type = other DNA
atcggacccaggattctttt

SEQ ID NO: 151 - DNA, 20 bp, synthetic construct
  source 1..20: mol_type = other DNA
ataagctggtcaccccacac

SEQ ID NO: 152 - DNA, 20 bp, synthetic construct
  source 1..20: mol_type = other DNA
gcacctgatgaaatgggaaa

SEQ ID NO: 153 - DNA, 20 bp, synthetic construct
  source 1..20: mol_type = other DNA
gggaatccctcctctctgaa

SEQ ID NO: 154-160 - skipped (7 entries)

SEQ ID NO: 161 - DNA, 21 bp, synthetic construct
  source 1..21: mol_type = other DNA
attgtgagcggataacaattc

SEQ ID NO: 162 - DNA, 46 bp, synthetic construct
  source 1..46: mol_type = other DNA
ttcaggttggaccggtgccaccatggggatacagggattgctacaa

SEQ ID NO: 163 - DNA, 51 bp, synthetic construct
  source 1..51: mol_type = other DNA
ggaggcggaggctcaggtggaggcggatcagacaagaagtacagcatcggc

SEQ ID NO: 164 - DNA, 50 bp, synthetic construct
  source 1..50: mol_type = other DNA
cacctgctccagcgcccgcaccagctcccgacaagaagtacagcatcggc

SEQ ID NO: 165 - DNA, 52 bp, synthetic construct
  source 1..52: mol_type = other DNA
agccgctaaggaggcagctgccgctaaggccgacaagaagtacagcatcggc

SEQ ID NO: 166 - DNA, 44 bp, synthetic construct
  source 1..44: mol_type = other DNA
ggccttagcggctgcagcctctgctcttgaatgggcaggcatag

SEQ ID NO: 167 - DNA, 49 bp, synthetic construct
  source 1..49: mol_type = other DNA
ggcagctgcctccttagcggctgcagcctctgctcttgaatgggcaggc

SEQ ID NO: 168 - DNA, 45 bp, synthetic construct
  source 1..45: mol_type = other DNA
gagcagaggctgcagccgctaaggccgactataaggaccacgacg

SEQ ID NO: 169 - DNA, 49 bp, synthetic construct
  source 1..49: mol_type = other DNA
agccgctaaggaggcagctgccgctaaggccgactataaggaccacgac

SEQ ID NO: 170 - DNA, 49 bp, synthetic construct
  source 1..49: mol_type = other DNA
ggagctggtgcgggcgctggagcaggtgctcttgaatgggcaggcatag

SEQ ID NO: 171 - DNA, 49 bp, synthetic construct
  source 1..49: mol_type = other DNA
cacctgctccagcgcccgcaccagctcccgactataaggaccacgacgg

SEQ ID NO: 172 - DNA, 49 bp, synthetic construct
  source 1..49: mol_type = other DNA
tgatccgcctccacctgagcctccgcctcctcttgaatgggcaggcata

SEQ ID NO: 173 - DNA, 49 bp, synthetic construct
  source 1..49: mol_type = other DNA
ggaggcggaggctcaggtggaggcggatcagactataaggaccacgacg

SEQ ID NO: 174 - DNA, 47 bp, synthetic construct
  source 1..47: mol_type = other DNA
tccttatagtctgagcctccgcctcctcttgaatgggcaggcatagc

SEQ ID NO: 175 - DNA, 45 bp, synthetic construct
  source 1..45: mol_type = other DNA
ccattcaagaggaggcggaggctcagactataaggaccacgacgg

SEQ ID NO: 176 - DNA, 42 bp, synthetic construct
  source 1..42: mol_type = other DNA
gaattctaactagagcttactttttcttttttgcctggccgg

SEQ ID NO: 177 - DNA, 21 bp, synthetic construct
  source 1..21: mol_type = other DNA
ctggagcacctgcctgaaatc

SEQ ID NO: 178 - DNA, 21 bp, synthetic construct
  source 1..21: mol_type = other DNA
cagaggattacatcaacgggt

SEQ ID NO: 179 - DNA, 19 bp, synthetic construct
  source 1..19: mol_type = other DNA
tcgagggcgacctgaaccc

SEQ ID NO: 180 - DNA, 20 bp, synthetic construct
  source 1..20: mol_type = other DNA
cctgaccttccgcatcccct

SEQ ID NO: 181 - DNA, 20 bp, synthetic construct
  source 1..20: mol_type = other DNA
ggcgatagcctgcacgagca

SEQ ID NO: 182 - DNA, 20 bp, synthetic construct
  source 1..20: mol_type = other DNA
tcgtgggaaccgccctgatc

SEQ ID NO: 183 - DNA, 22 bp, synthetic construct
  source 1..22: mol_type = other DNA
caactagaaggcacagtcgagg

SEQ ID NO: 184 - DNA, 36 bp, synthetic construct
  source 1..36: mol_type = other DNA
gaaggagatataccatgggcatccaaggccttttac

SEQ ID NO: 185 - DNA, 42 bp, synthetic construct
  source 1..42: mol_type = other DNA
ctattgagtatttcttatcggccgcaggaactccgtggatgc

SEQ ID NO: 186 - DNA, 60 bp, synthetic construct
  source 1..60: mol_type = other DNA
gataagaaatactcaataggcttagctatgccggctcactctcgtggtggtggtggttct

SEQ ID NO: 187 - DNA, 25 bp, synthetic construct
  source 1..25: mol_type = other DNA
gataagaaatactcaataggcttag

SEQ ID NO: 188 - DNA, 54 bp, synthetic construct
  source 1..54: mol_type = other DNA
ctattgagtatttcttatcagaaccaccaccaccacgagagtgagccggcatag

SEQ ID NO: 189 - DNA, 38 bp, synthetic construct
  source 1..38: mol_type = other DNA
aacttcttcaaaattccacggggtaattgtttcttcag

SEQ ID NO: 190 - DNA, 38 bp, synthetic construct
  source 1..38: mol_type = other DNA
gaaggagatataccatgatgggtatccagggtctgctg

SEQ ID NO: 191 - DNA, 69 bp, synthetic construct
  source 1..69: mol_type = other DNA
ctattgagtatttcttatccggagccggagccggagccggagccggagcacgagagtgagccggcatag

SEQ ID NO: 192-200 - skipped (9 entries)

SEQ ID NO: 201 - DNA, 1452 bp, synthetic construct
  source 1..1452: mol_type = other DNA
cactaaggcgcagaagaaggatggtaagaagcgtaagcgcagccgcaaggagagctattctatctatgtgtacaaggttctgaagcaggtccaccccgacaccggcatctcatccaaggccatggggatcatgaattccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtcggagggcactaaagcagttaccaagtacactagctctaaagtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggctaccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtaagtgcttatgtaagcacttccaaacccaaaggctcttttcagagccacctactttgtcacaaggagagctataaccacaatttcttaaggtggtgctgctgctattctgtttcagttctagaggatcaactggaatgttagcgaagacaagttttagagccaaggttaacttggacggggccgtgcgcggtgcctcttgcctttaatcccggcaatttgggaggccgaggcgggcggatcacgaggtcaggagatggagaccatcctgcttaacacgatgaaaccccgtctctactaaaaatacaaaataattagctgggcgtgatggtgggcgcctgtagtcccagctactcgggaggctgaggcaggagaatggcgtgaacgcgggaggcggagcttgcagtgagccgagatcgcgccatg

SEQ ID NO: 202 - DNA, 2231 bp, synthetic construct
  source 1..2231: mol_type = other DNA
ccatagacggagcaggacattcccgaaagtaagaggaggaaggcatccaccctaggtacaatacttgtatatatggggagatgtgctctgctacaagtttgtgataaaggattaattttcttagttactatattttgcaagaatcaacattattatctttaaacaaaattaagaatgcctttgttctccagatatagggatatctggacactcctaagtctgagtctgtttagtaaacattatttatttgttcccttaaccgtaaacatctagaagctaggaatgactgactttctgggaatgcagcccagaaagtctcagcctcattttcctagccctcactcaaaatggagttactctggttcaagtaactctgacacttttcttctctttttttcttcttttttccttcctttattttttattttttatttttgaaataagaaatcaagaatacttgatgtttcatctaaaacaatacccataattgataagccaaaacaaaaacctaggtcttctaactcaaaactaggatgttttgctgtctctgctgatactcggctgatcgttaataggtaattaacaaacaagccttgctatgtccccctcagtttattaccattagatcatatgcctactgtcaatcatattaatccacaactatgcatttcacaaaacttgccataaaaattcacaggtttcccgcttccctcgagttttcatttccgaagggtcccatgtaatataaaacttatattaaatacatttgtatgcttttctcttgctaatctttttttttgttttttgagactgagccttgctctgtcacccaggctggagtgggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtccaatggcgcgatctcggctcactgcaacctccgcttcccaggttcaagcgattctactgcctcgccctcccgagtagctgggaccacagatacgtgccacc

SEQ ID NO: 203 - DNA, 3624 bp, synthetic construct
  source 1..3624: mol_type = other DNA
ttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtgcggccactcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataatgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacagtactaagctttagggacaggattcgtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctct

SEQ ID NO: 204-210 - skipped (7 entries)

SEQ ID NO: 211 - protein, 7 aa, synthetic construct
GLLKPVL

SEQ ID NO: 212 - protein, 10 aa, synthetic construct
GGGGSGGGGS

SEQ ID NO: 213 - protein, 8 aa, synthetic construct
AEAAAAKA

SEQ ID NO: 214 - protein, 14 aa, synthetic construct
AEAAAAKEAAAAKA

SEQ ID NO: 215 - protein, 10 aa, synthetic construct
APAPAPAPAP

SEQ ID NO: 216-220 - skipped (5 entries)

SEQ ID NO: 221 - protein, 7 aa, Simian virus 40
PKKKRKV

SEQ ID NO: 222 - protein, 16 aa, unidentified
  source 1..16: note = Nucleoplasmin NLS sequence
KRPAATKKAGQAKKKK

SEQ ID NO: 223 - DNA, 132 bp, synthetic construct
  source 1..132: mol_type = other DNA
caaacagacaccatggtgcacctgactcctgaggagaagtctgccgttactgccctgtggggcaaggtgaacgtggatgaagttggtggtgaggccctgggcaggttggtatcaaggttacaagacaggttt

SEQ ID NO: 224 - DNA, 132 bp, synthetic construct
  source 1..132: mol_type = other DNA
nannancnnnnntattctnnnnangaatannaanncctgaaccnnnnaagtctnnnnnancccnnaaaaanngnnnnaagaangctatnantaangcncanaagaangannnnaagaannnnaancnnagnc

SEQ ID NO: 225 - DNA, 130 bp, synthetic construct
  source 1..130: mol_type = other DNA
gacggctgtgcgcctgctgctgcctggggagctggctaagcatgctgtgtcggngggcactaaagnagtnaccaagtacactagctctaaagngagcaagggcgaggagganaacanggnctntntccca

SEQ ID NO: 226 - DNA, 129 bp, synthetic construct
  source 1..129: mol_type = other DNA
ntttnncctttnnngatnnnatnggcatgnacnanctntacaagtaantgcttatgtaagnacttccaaacccaaaggctnttttcanagccacctactttgtcacanngagagctataaccacaattt

SEQ ID NO: 227 - DNA, 130 bp, synthetic construct
  source 1..130: mol_type = other DNA
tgtnntnnnngctnnnnggnnnnnngnggnnnnnnnnnnnnnnnnnnnnnnnnnnnggngcttnnngtgagncgngntcgngnnnntgnnntnnngnnngggngnnnnnnnnnnnnnnnnntnnnnnnnn

SEQ ID NO: 228 - DNA, 683 bp, synthetic construct
  source 1..683: mol_type = other DNA
gtgctttatccaatagaaaaagataacataaattccatatttgcataaaccccacccctcagtgaaaccgtgtttcttttgtccaatcagaagtgaggaatcttaaaccgtcatttgaatctcaggactataaatacatgggctctgaactgttctctgtactactctgtagtggagagtgttagtagcttttctattctgtttaggaatagcaatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaattccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtcggagggcactaaagcagttaccaagtacactagctctaaagtgagcaagggcgangaggataacatggcctctctcccagcgacacatgagttacacatctttggctccatcaacggtgtggactttgaca

SEQ ID NO: 229 - DNA, 708 bp, synthetic construct
  source 1..708: mol_type = other DNA
caataggaagctatcctattggtcaattatgtttggtgctttatccaatagaaaaagataacataaattccatatttgcataaaccccacccctcagtgaaaccgtgtttcttttgtccaatcagaagtgaggaatcttaaaccgtcatttgaatctcaggactataaatacatgggctctgaactgttctctgtactactctgtagtggagagtgttagtagcttttctattctgtttaggaatagcaatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaattccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtcggagggcactaaagcagttaccaagtacactagctctaaagtgagcaagggcgaggaggataacatggcctctctcccagcgacacatgagttacacatctttggctccatcaacggtgtg

SEQ ID NO: 230 - DNA, 717 bp, synthetic construct
  source 1..717: mol_type = other DNA
ctgcaataggaagctatcctattggtcaattatgtttggtgctttatccaatagaaaaagataacataaattccatatttgcataaaccccacccctcagtgaaaccgtgtttcttttgtccaatcagaagtgaggaatcttaaaccgtcatttgaatctcaggactataaatacatgggctctgaactgttctctgtactactctgtagtggagagtgttagtagcttttctattctgtttaggaatagcaatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaattccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtcggagggcactaaagcagttaccaagtacactagctctaaagtgagcaagggcgaggaggataacatggcctctctcccagcgacacatgagttacacatctttggctccatcaacggtgtggacttt

SEQ ID NO: 231 - DNA, 742 bp, synthetic construct
  source 1..742: mol_type = other DNA
caaactgcaaggctgcaataggaagctatcctattggtcaattatgtttcgtgctttatccaatagaaaaagataacataaattccatatttgcataaaccccacccctcagtgaaaccgtgtttcttttgtccaatcagaagtgaggaatcttaaaccgtcatttgaatctcaggactataaatacatgggctctgaactgttctctgtactactctgtagtggagagtgttagtagcttttctattctgtttaggaatagcaatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaattccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtcggagggcactaaagcagttaccaagtacactagctctaaagtgagcaagggcgaggaggataacatggcctctctcccagcgacacatgagttacacatctttggctccatcaacggtgtggactttgacatggtgggtc

SEQ ID NO: 232 - DNA, 394 bp, synthetic construct
  source 1..394: mol_type = other DNA
agtgcttatgtagcacttccaaacccaaaggctcttttcagagccacctactttgtcacaaggagagctataaccacaatttcttaaggtggtgctgctgctattctgtttcagttctagaggatcaactggaatgttagcgaagacaagttttagagccaaggttaacttggacggggccgtgcgcggtgcctcttgcctttaatcccggcaatttgggaggccgaggcgggcggatcacgaggtcaggagatggagaccatcctgcttaacacgatgaaaccccgtctctactaaaaatacaaaataattagctgggcgtgatggtgggcgcctgtantcccagctactcgggaggctgaggcaggagaatggcgtgaacgcatgaggcgga

SEQ ID NO: 233 - DNA, 481 bp, synthetic construct
  source 1..481: mol_type = other DNA
ccgatgtgatgggcatggacgagctgtacaagtaagtgcttatgtaagcacttccaaacccaaaggctcttttcagagccacctactttgtcacaaggagagctataaccacaatttcttaaggtggtgctgctgctattctgtttcagttctagaggatcaactggaatgttagcgaagacaagttttagagccaaggttaacttggacggggccgtgcgcggtgcctcttgcctttaatcccggcaatttgggaggccgaggcgggcggatcacgaggtcaggagatggagaccatcctgcttaacacgatgaaaccccgtctctactaaaaatacaaaataattagctgggcgtgatggtgggcgcctgtagtcccagctactcgggaggctgaggcaggagaatggcgtgaacgcaggaggcggagcttgcagtgagccgagatcgcgccactgcactccagcctgggtgacagagc

SEQ ID NO: 234 - DNA, 417 bp, synthetic construct
  source 1..417: mol_type = other DNA
ccgatgtgatgggcatggacgagctgtacaagtaagtgcttatgtaagcacttccaaacccaaaggctcttttcagagccacctactttgtcacaaggagagctataaccacaatttcttaaggtggtgctgctgctattctgtttcagttctagaggatcaactggaatgttagcgaagacaagttttagagccaaggttaacttggacggggccgtgcgcggtgcctcttgcctttaatcccggcaatttgggaggccgaggcgggcggatcacgaggtcaggagatggagaccatcctgcttaacacgatgaaaccccgtctctactaaaaatacaaaataattagctgggcgtgatggtgggcgcctgtagtcccagctactcgggaggctgaggcaggagaatggcgtgaac

SEQ ID NO: 235 - DNA, 500 bp, synthetic construct
  source 1..500: mol_type = other DNA
aggcctttaccgatgtgatgggcatggacgagctgtacaagtaagtgcttatgtaagcacttccaaacccaaaggctcttttcagagccacctactttgtcacaaggagagctataaccacaatttcttaaggtggtgctgctgctattctgtttcagttctagaggatcaactggaatgttagcgaagacaagttttagagccaaggttaacttggacggggccgtgcgcggtgcctcttgcctttaatcccggcaatttgggaggccgaggcgggcggatcacgaggtcaggagatggagaccatcctgcttaacacgatgaaaccccgtctctactaaaaatacaaaataattagctgggcgtgatggtgggcgcctgtagtcccagctactcgggaggctgaggcaggagaatggcgtgaacgcgggaggcggagcttgcagtgagccgagatcgcgccatggcactccagcctgggtgacagagcgagactccgt

SEQ ID NO: 236 - protein, 6 aa, synthetic construct
HHHHHH